SEQUENCE LISTING (48 sequences)

SEQ ID NO: 1 - protein, 124 aa, Homo sapiens
QVQLVESGGGVVQPGRSLRLSCAASGFTFSSYAMHWVRQAPGKGLEWVAVISYDGNNKYYADSVKGRFTISRDNSKNTLYLQMNSLRAEDTAVYYCARAVPWGSRYYYYGMDVWGQGTTVTVSS

SEQ ID NO: 2 - protein, 119 aa, Homo sapiens
EVQLVQSGAEVKKPGESLKISCKGSGYSFTSNWIGWVRQMPGKGLEWMGIIYPGDSDTRYSPSFQGQVTISADKSISTAYLQWSSLKASDTAMYYCARQTGFLWSSDLWGRGTLVTVSS

SEQ ID NO: 3 - protein, 119 aa, Homo sapiens
EVQLVQSGAEVKKPGESLKISCKGSGYSFTSNWIGWVRQMPGKGLEWMGIIYPGDSDTRYSPSFQGQVTISADKSISTAYLQWNSLKASDTAMYYCARQTGFLWSFDLWGRGTLVTVSS

SEQ ID NO: 4 - protein, 121 aa, Homo sapiens
EVQLVQSGSEVKKPGESLKISCKGSGYSFTNYWIGWVRQMPGKGLEWMGIIYPGDSDTRYSPSFQGQVTISADKSISTAYLQWSSLKASDTAMYYCASPGYTSSWTSFDYWGQGTLVTVSS

SEQ ID NO: 5 - protein, 107 aa, Homo sapiens
AIQLTQSPSSLSASVGDRVTITCRASQGISSALAWYQQKSGKAPKLLIFDASSLESGVPSRFSGSGSGTDFTLTISSLQPEDFATYYCQQFNSYPLTFGGGTKVEIK

SEQ ID NO: 6 - protein, 107 aa, Homo sapiens
AIQLTQSPSSLSASVGDRVTITCRASQDISSALAWYQQKPGKAPKLLIYDASSLESGVPSRFSGYGSGTDFTLTINSLQPEDFATYYCQQFNSYPLTFGGGTKVEIK

SEQ ID NO: 7 - protein, 109 aa, Homo sapiens
AIQLTQSPSSLSASVGDRVTITCRASQDISSALAWYQQKPGKAPKLLIYDASSLESGVPSRFSGSGSGTDFTLTISSLQPEDFATYYCQQFNSYPLTFGGGTKVEIKIK

SEQ ID NO: 8 - protein, 108 aa, Homo sapiens
EIVLTQSPATLSLSPGERATLSCRASQSVSSYLAWYQQKPGQAPRLLIYDASNRATGIPARFSGSGSGTDFTLTISSLEPEDFAVYYCQQRSNWPLFTFGPGTKVDIK

SEQ ID NO: 9 - protein, 5 aa, Homo sapiens
SYAMH

SEQ ID NO: 10 - protein, 5 aa, Homo sapiens
SNWIG

SEQ ID NO: 11 - protein, 5 aa, Homo sapiens
SNWIG

SEQ ID NO: 12 - protein, 5 aa, Homo sapiens
NYWIG

SEQ ID NO: 13 - protein, 17 aa, Homo sapiens
VISYDGNNKYYADSVKG

SEQ ID NO: 14 - protein, 17 aa, Homo sapiens
IIYPGDSDTRYSPSFQG

SEQ ID NO: 15 - protein, 17 aa, Homo sapiens
IIYPGDSDTRYSPSFQG

SEQ ID NO: 16 - protein, 17 aa, Homo sapiens
IIYPGDSDTRYSPSFQG

SEQ ID NO: 17 - protein, 15 aa, Homo sapiens
AVPWGSRYYYYGMDV

SEQ ID NO: 18 - protein, 10 aa, Homo sapiens
QTGFLWSSDL

SEQ ID NO: 19 - protein, 10 aa, Homo sapiens
QTGFLWSFDL

SEQ ID NO: 20 - protein, 12 aa, Homo sapiens
PGYTSSWTSFDY

SEQ ID NO: 21 - protein, 11 aa, Homo sapiens
RASQGISSALA

SEQ ID NO: 22 - protein, 11 aa, Homo sapiens
RASQDISSALA

SEQ ID NO: 23 - protein, 11 aa, Homo sapiens
RASQDISSALA

SEQ ID NO: 24 - protein, 11 aa, Homo sapiens
RASQSVSSYLA

SEQ ID NO: 25 - protein, 7 aa, Homo sapiens
DASSLES

SEQ ID NO: 26 - protein, 7 aa, Homo sapiens
DASSLES

SEQ ID NO: 27 - protein, 7 aa, Homo sapiens
DASSLES

SEQ ID NO: 28 - protein, 7 aa, Homo sapiens
DASNRAT

SEQ ID NO: 29 - protein, 9 aa, Homo sapiens
QQFNSYPLT

SEQ ID NO: 30 - protein, 9 aa, Homo sapiens
QQFNSYPLT

SEQ ID NO: 31 - protein, 9 aa, Homo sapiens
QQFNSYPLT

SEQ ID NO: 32 - protein, 10 aa, Homo sapiens
QQRSNWPLFT

SEQ ID NO: 33 - DNA, 372 bp, Homo sapiens
  CDS (1)..(372)
caggtgcaactggtggagtctgggggaggcgtggtccagcctgggaggtccctgagactctcctgtgcagcctctggattcaccttcagtagctatgctatgcactgggtccgccaggctccaggcaaggggctggagtgggtggcagttatatcatatgatggaaacaataaatactacgcagactccgtgaagggccgattcaccatctccagagacaattccaagaacacgctgtatctgcaaatgaacagcctgagagctgaggacacggctgtgtattactgtgcgagagccgtcccctggggatcgaggtactactactacggtatggacgtctggggccaagggaccacggtcaccgtctcctca

SEQ ID NO: 34 - DNA, 357 bp, Homo sapiens
  CDS (1)..(357)
gaggtgcagctggtgcagtctggagcagaggtgaaaaagcccggggagtctctgaagatctcctgtaagggttctggatacagctttaccagtaactggatcggctgggtgcgccagatgcccgggaaaggcctggagtggatggggatcatctatcctggtgactctgataccagatacagcccgtccttccaaggccaggtcaccatctcagccgacaagtccatcagcaccgcctacctgcagtggagcagcctgaaggcctcggacaccgccatgtattactgtgcgaggcaaactggtttcctctggtcctccgatctctggggccgtggcaccctggtcactgtctcctca

SEQ ID NO: 35 - DNA, 357 bp, Homo sapiens
  CDS (1)..(357)
gaggtgcagctggtgcagtctggagcagaggtgaaaaagcccggggagtctctgaagatctcctgtaagggttctggatacagttttaccagcaactggatcggctgggtgcgccagatgcccgggaaaggcctggagtggatggggatcatctatcctggtgactctgataccagatacagcccgtccttccaaggccaggtcaccatctcagccgacaagtccatcagcaccgcctacctgcagtggaacagcctgaaggcctcggacaccgccatgtattactgtgcgagacaaactggtttcctctggtccttcgatctctggggccgtggcaccctggtcactgtctcctca

SEQ ID NO: 36 - DNA, 363 bp, Homo sapiens
  CDS (1)..(363)
gaggtgcagctggtgcagtctggatcagaggtgaaaaagcccggggagtctctgaagatctcctgtaagggttctggatacagctttaccaactactggatcggctgggtgcgccagatgcccgggaaaggcctggagtggatggggatcatctatcctggtgactctgataccagatacagcccgtccttccaaggccaggtcaccatctcagccgacaagtccatcagcaccgcctatctgcagtggagcagcctgaaggcctcggacaccgccatgtattactgtgcgagtcccgggtataccagcagttggacttcttttgactactggggccagggaaccctggtcaccgtctcctca

SEQ ID NO: 37 - DNA, 321 bp, Homo sapiens
  CDS (1)..(321)
gccatccagttgacccagtctccatcctccctgtctgcatctgtaggagacagagtcaccatcacttgccgggcaagtcagggcattagcagtgctttagcctggtatcagcagaaatcagggaaagctcctaagctcctgatctttgatgcctccagtttggaaagtggggtcccatcaaggttcagcggcagtggatctgggacagatttcactctcaccatcagcagcctgcagcctgaagattttgcaacttattactgtcaacagtttaacagttatcctctcactttcggcggagggaccaaggtggagatcaaa

SEQ ID NO: 38 - DNA, 321 bp, Homo sapiens
  CDS (1)..(321)
gccatccagttgacccagtctccatcctccctgtctgcatctgtaggagacagagtcaccatcacttgccgggcaagtcaggacattagcagtgctttagcctggtatcaacagaaaccagggaaagctcctaagctcctgatctatgatgcctccagtttggaaagtggggtcccatcaaggttcagcggctatggatctgggacagatttcactctcaccatcaacagcctgcagcctgaagattttgcaacttattactgtcaacagtttaatagttacccgctcactttcggcggagggaccaaggtggagatcaaa

SEQ ID NO: 39 - DNA, 327 bp, Homo sapiens
  CDS (1)..(327)
gccatccagttgacccagtctccatcctccctgtctgcatctgtaggagacagagtcaccatcacttgccgggcaagtcaggacattagcagtgctttagcctggtatcagcagaaaccggggaaagctcctaagctcctgatctatgatgcctccagtttggaaagtggggtcccatcaaggttcagcggcagtggatctgggacagatttcactctcaccatcagcagcctgcagcctgaagattttgcaacttattactgtcaacagtttaatagttacccgctcactttcggcggagggaccaaggtggagatcaaaatcaaa

SEQ ID NO: 40 - DNA, 324 bp, Homo sapiens
  CDS (1)..(324)
gaaattgtgttgacacagtctccagccaccctgtctttgtctccaggggaaagagccaccctctcctgcagggccagtcagagtgttagcagctacttagcctggtaccaacagaaacctggccaggctcccaggctcctcatctatgatgcatccaacagggccactggcatcccagccaggttcagtggcagtgggtctgggacagacttcactctcaccatcagcagcctagagcctgaagattttgcagtttattactgtcagcagcgtagcaactggcccctattcactttcggccctgggaccaaagtggatatcaaa

SEQ ID NO: 41 - protein, 98 aa, Homo sapiens
QVQLVESGGGVVQPGRSLRLSCAASGFTFSSYAMHWVRQAPGKGLEWVAVISYDGSNKYYADSVKGRFTISRDNSKNTLYLQMNSLRAEDTAVYYCAR

SEQ ID NO: 42 - protein, 98 aa, Homo sapiens
EVQLVQSGAEVKKPGESLKISCKGSGYSFTSYWIGWVRQMPGKGLEWMGIIYPGDSDTRYSPSFQGQVTISADKSISTAYLQWSSLKASDTAMYYCAR

SEQ ID NO: 43 - protein, 95 aa, Homo sapiens
AIQLTQSPSSLSASVGDRVTITCRASQGISSALAWYQQKPGKAPKLLIYDASSLESGVPSRFSGSGSGTDFTLTISSLQPEDFATYYCQQFNSYP

SEQ ID NO: 44 - protein, 94 aa, Homo sapiens
EIVLTQSPATLSLSPGERATLSCRASQSVSSYLAWYQQKPGQAPRLLIYDASNRATGIPARFSGSGSGTDFTLTISSLEPEDFAVYYCQQRSNW

SEQ ID NO: 45 - protein, 20 aa, Homo sapiens
YYYYYGMDVWGQGTTVTVSS

SEQ ID NO: 46 - protein, 12 aa, Homo sapiens
LTFGGGTKVEIK

SEQ ID NO: 47 - protein, 12 aa, Homo sapiens
FTFGPGTKVDIK

SEQ ID NO: 48 - protein, 15 aa, Artificial Sequence
  Description of Artificial Sequence Synthetic Gly-Ser linker
GGGGSGGGGSGGGGS

(19) United States
(12) Patent Application Publication
(10) Pub. No.: US 2009/0297438 A1
(43) Pub. Date: Dec. 3, 2009

(54) Human Monoclonal Antibodies to Prostate Specific Membrane Antigen (PSMA)

(76) Inventors: Haichun Huang, Fremont,, CA (US); David John king, Belmont, CA (US); Chin Pan, Los Altos, CA (US); Josephine M. Cardarelli, San Carlos, CA (US)

Correspondence Address: LAHIVE & COCKFIELD, LLP;FLOOR 30, SUITE 3000, ONE POST OFFICE SQUARE, BOSTON, MA, 02109, US

(21) Appl. No.: 11/918,153

(22) PCT Filed: Feb. 17, 2006
(86) PCT No.: PCT/US2006/005852

§ 371 Date: Jan. 30, 2009

Related U.S. Application Data
(60) Provisional application No. 60/654,125, filed on Feb. 18, 2005.
(60) Provisional application No. 60/720,499, filed on Sep. 26, 2005.
(60) Provisional application No. 60/748,417, filed on Dec. 8, 2005.

(51) Int. Cl. A61K 51/10; C07K 16/28; A61K 39/395; C07H 21/00; C12N 15/63; C12N 5/00; A01K 67/027; C07K 1/107; A61K 38/20; C12Q 1/02; A61P 35/00

(52) U.S. Cl. 424/1.49; 530/388.15; 530/388.2; 424/142.1; 424/152.1; 530/391.7; 424/178.1; 530/387.3; 424/136.1; 536/23.53; 435/320.1; 435/325; 800/18; 530/402; 424/85.2; 435/29; 800/3

(57) ABSTRACT

The present invention provides isolated monoclonal antibodies, particularly human monoclonal antibodies, that specifically bind to PSMA with high affinity. Nucleic acid molecules encoding the antibodies of the invention, expression vectors, host cells and methods for expressing the antibodies of the invention are also provided. Immunoconjugates, bispecific molecules and pharmaceutical compositions comprising the antibodies of the invention are also provided. The invention also provides methods for treating cancer.

### BACKGROUND OF THE INVENTION

Prostate cancer is a leading cause of morbidity and mortality among men. Treatments for prostate cancer include surgery, hormones, radiation, and chemotherapy. There is little effective treatment for metastatic prostate disease. Therefore, the identification of genes and/or gene products that represent diagnostic and prognostic markers, as well as targets for therapy, is critical. Prostate specific antigen (PSA) is one such cancer marker which is useful in the clinical diagnosis and staging of prostate cancer. However, PSA cannot differentiate benign prostatic hyperplasia (BPH) from prostatitis or prostate cancer in the range of 4-10 ng/ml, thus, necessitating a cytologic and/or histologic assessment to confirm the proper diagnosis (Barren, R. J. et al. (1998) Prostate 36:181-188).

Prostate specific membrane antigen (PSMA) is a 750 amino acid, type II transmembrane glycoprotein of approximately 110 kD that has 54% homology to the transferrin receptor. PSMA has 3 structural domains, including a 19 amino acid intracellular domain, a 24 amino acid transmembrane domain, and a 707 amino acid extracellular domain. The PSMA protein displays neurocarboxypeptidase and folate hydrolase activity and is reported to be involved in the neuroendocrine regulation of prostate growth and differentiation (Heston, W. D. (1996) Urologe-Ausgabe A. 35:400-407). PSM′ is an alternatively spliced form of PSMA which is localized in the cytoplasm.

PSMA is predominantly expressed by prostatic epithelial cells. The expression of PSMA is increased in prostate cancer, especially in poorly differentiated, metastatic, and hormone refractory carcinomas (Gregorakis, A. K. et al. (1998) Seminars in Urologic Oncology 16:2-12; Silver, D. A. (1997) Clinical Cancer Research 3:81-85). Low level expression of PSMA is observed in extraprostatic tissues such as the small bowel, salivary gland, duodenal mucosa, proximal renal tubules, and brain (Silver, D. A. (1997) Clinical Cancer Research 3:81-85). PSMA is also expressed in endothelial cells of capillary vessels in peritumoral and endotumoral areas of certain malignancies, including renal cell carcinomas, and colon carcinomas, but not in blood vessels from normal tissues. In addition, PSMA is reported to be related to tumor angiogenesis (Silver, D. A. (1997) Clinical Cancer Research 3:81-85). Recently, PSMA has been demonstrated to be expressed in endothelial cells of tumor-associated neovasculature in carcinomas of the colon, breast, bladder, pancreas, kidney, and melanoma (Chang, S. S. (2004) Curr Opin Investig Drugs 5:611-5).

Antibodies against the extracellular domain of PSMA have been described (see e.g., Liu, H. et al. (1997) Cancer Res. 57:3629-3634; Murphy, G. P. et al. (1998) J. Urol. 160:2396-2401; Wang, S. et al. (2001) Int. J. Cancer 92:871-876; Kato, K. et al. (2003) Int. J. Urol. 10:439-444; U.S. Pat. No. 6,150,508 and U.S. Pat. No. 6,107,090). More recently, human and humanized antibodies that bind PSMA have been described (see e.g., Bander, N. H. et al. (2003) Semin. Oncol. 30:667-676; PCT Publication WO 02/098897; PCT Publication WO 01/09192; PCT Publication WO 03/064606; PCT Publication WO 03/034903; and US Application No. 2004/0033229). Such antibodies have been used for imaging of prostate cancer cells (see e.g., Yao, D. et al. (2002) Semin. Urol. Oncol. 20:211-218; Bander, N. H. et al. (2003) J. Urol. 170:1717-1721). Anti-PSMA antibodies also have been used for therapeutic intervention in treatment of prostate cancer, typically as a conjugate with a chemotherapeutic agent or radioactive isotope (see e.g. Nanus, D. M. et al. (2003) J. Urol. 170:S84-89; Milowsky, M. I. et al. (2004) J. Clin. Oncol. 22:2522-2531; Henry, M. D. et al. (2004) Cancer Res. 64:7995-8001).

Accordingly, PSMA represents a valuable target for the treatment of prostate cancer and a variety of other diseases characterized by PSMA expression and additional therapeutic agents that recognize PSMA are desired.

### SUMMARY OF THE INVENTION

The present invention provides isolated monoclonal antibodies, in particular human monoclonal antibodies, that bind to PSMA. The antibodies of the invention have desired properties, such as high affinity for PSMA, the ability to be internalized by PSMA expressing cells and a high melting temperature. Preferably, antibodies of the invention have melting temperatures of at least 67° C., or at least 69° C. or at least 71° C.

In one aspect, the invention pertains to an isolated monoclonal antibody, or an antigen-binding portion thereof, comprising a heavy chain variable region that is the product of or derived from a human V_{H} 3-30.3 gene, wherein the antibody specifically binds PSMA. The invention further provides an isolated monoclonal antibody, or an antigen-binding portion thereof, comprising a light chain variable region that is the product of or derived from a human V_{K} L18 gene, wherein the antibody specifically binds PSMA.

In a preferred embodiment, the invention provides an isolated monoclonal antibody, or an antigen-binding portion thereof, comprising:
(a) a heavy chain variable region of a human V_{H} 3-30.3 gene; and
(b) a light chain variable region of a human V_{K} L18 gene;

wherein the antibody specifically binds PSMA.

In another preferred embodiment, the invention provides an isolated monoclonal antibody, or an antigen-binding portion thereof, comprising:
(a) a heavy chain variable region of a human V_{H} 5-51 gene; and
(b) a light chain variable region of a human V_{K} L18 gene;

wherein the antibody specifically binds PSMA.

In preferred embodiments, the invention provides an isolated monoclonal antibody, or antigen binding portion thereof, comprising:
(a) a heavy chain variable region CDR1 comprising an amino acid sequence selected from the group consisting of SEQ ID NOs: 9, 10, 11, and 12;
(b) a heavy chain variable region CDR2 comprising an amino acid sequence selected from the group consisting of SEQ ID NOs: 13, 14, 15, and 16;
(c) a heavy chain variable region CDR3 comprising an amino acid sequence selected from the group consisting of SEQ ID NOs: 17, 18, 19, and 20;
(d) a light chain variable region CDR1 comprising an amino acid sequence selected from the group consisting of SEQ ID NOs: 21, 22, 23, and 24;
(e) a light chain variable region CDR2 comprising an amino acid sequence selected from the group consisting of SEQ ID NOs: 25, 26, 27, and 28; and
(f) a light chain variable region CDR3 comprising an amino acid sequence selected from the group consisting of SEQ ID NOs: 29, 30, 31, and 32;

wherein the antibody specifically binds PSMA.

A preferred combination comprises:
(a) a heavy chain variable region CDR1 comprising SEQ ID NO: 9;
(b) a heavy chain variable region CDR2 comprising SEQ ID NO: 13;
(c) a heavy chain variable region CDR3 comprising SEQ ID NO: 17;
(d) a light chain variable region CDR1 comprising SEQ ID NO: 21;
(e) a light chain variable region CDR2 comprising SEQ ID NO: 25; and

(f) a light chain variable region CDR3 comprising SEQ ID NO: 29.

Another preferred combination comprises:
(a) a heavy chain variable region CDR1 comprising SEQ ID NO: 10;
(b) a heavy chain variable region CDR2 comprising SEQ ID NO: 14;
(c) a heavy chain variable region CDR3 comprising SEQ ID NO: 18;
(d) a light chain variable region CDR1 comprising SEQ ID NO: 22;
(e) a light chain variable region CDR2 comprising SEQ ID NO: 26; and
(f) a light chain variable region CDR3 comprising SEQ ID NO: 30.
   Another preferred combination comprises:
(a) a heavy chain variable region CDR1 comprising SEQ ID NO: 11;
(b) a heavy chain variable region CDR2 comprising SEQ ID NO: 15;
(c) a heavy chain variable region CDR3 comprising SEQ ID NO: 19;
(d) a light chain variable region CDR1 comprising SEQ ID NO: 23;
(e) a light chain variable region CDR2 comprising SEQ ID NO: 27; and
(f) a light chain variable region CDR3 comprising SEQ ID NO: 31.
   Another preferred combination comprises:
(a) a heavy chain variable region CDR1 comprising SEQ ID NO: 12;
(b) a heavy chain variable region CDR2 comprising SEQ ID NO: 16;
(c) a heavy chain variable region CDR3 comprising SEQ ID NO: 20;
(d) a light chain variable region CDR1 comprising SEQ ID NO: 24;
(e) a light chain variable region CDR2 comprising SEQ ID NO: 28; and
(f) a light chain variable region CDR3 comprising SEQ ID NO: 32.
   Other preferred antibodies of the invention, or antigen binding portions thereof, comprise:
(a) a heavy chain variable region comprising an amino acid sequence selected from the group consisting of SEQ ID NOs: 1, 2, 3, and 4; and
(b) a light chain variable region comprising an amino acid sequence selected from the group consisting of SEQ ID NOs: 5, 6, 7, and 8;

wherein the antibody specifically binds PSMA.

A preferred combination comprises:
(a) a heavy chain variable region comprising the amino acid sequence of SEQ ID NO: 1; and
(b) a light chain variable region comprising the amino acid sequence of SEQ ID NO: 5.
   Another preferred combination comprises:
(a) a heavy chain variable region comprising the amino acid sequence of SEQ ID NO: 2; and
(b) a light chain variable region comprising the amino acid sequence of SEQ ID NO: 6.
   Another preferred combination comprises:
(a) a heavy chain variable region comprising the amino acid sequence of SEQ ID NO: 3; and
(b) a light chain variable region comprising the amino acid sequence of SEQ ID NO: 7.
   Another preferred combination comprises:
(a) a heavy chain variable region comprising the amino acid sequence of SEQ D NO: 4; and
(b) a light chain variable region comprising the amino acid sequence of SEQ ID NO: 8.

The antibodies of the invention can be, for example, full-length antibodies, for example of an IgG1 or IgG4 isotype. Alternatively, the antibodies can be antibody fragments, such as Fab or Fab′2 fragments, or single chain antibodies.

The invention also provides an immunoconjugate comprising an antibody of the invention, or antigen-binding portion thereof, linked to a therapeutic agent, such as a cytotoxin or a radioactive isotope. The invention also provides a bispecific molecule comprising an antibody, or antigen-binding portion thereof, of the invention, linked to a second functional moiety having a different binding specificity than said antibody, or antigen binding portion thereof.

Compositions comprising an antibody, or antigen-binding portion thereof, or immunoconjugate or bispecific molecule of the invention and a pharmaceutically acceptable carrier are also provided.

Nucleic acid molecules encoding the antibodies, or antigen-binding portions thereof, of the invention are also encompassed by the invention, as well as expression vectors comprising such nucleic acids and host cells comprising such expression vectors. Moreover, the invention provides a transgenic mouse comprising human immunoglobulin heavy and light chain transgenes, wherein the mouse expresses an antibody of the invention, as well as hybridomas prepared from such a mouse, wherein the hybridoma produces the antibody of the invention.

In yet another aspect, the invention provides a method of inhibiting growth of tumor cells in a subject, wherein the tumor cells or vascular endothelial cells proximate to the tumor cells express PSMA, comprising administering to a subject an anti-PSMA human antibody of the present invention in an amount effective to growth of the tumor cells. In a preferred embodiment, growth of prostate tumor cells is inhibited.

The invention also provides methods for making “second generation” anti-PSMA antibodies based on the sequences of the anti-PSMA antibodies provided herein. For example, the invention provides a method for preparing an anti-PSMA antibody comprising:

(a) providing: (i) a heavy chain variable region antibody sequence comprising a CDR1 sequence that is selected from the group consisting of SEQ ID NOs: 9, 10, 11, and 12, a CDR2 sequence that is selected from the group consisting of SEQ ID NOs: 13, 14, 15, and 16; and a CDR3 sequence that is selected from the group consisting of SEQ ID NOs: 17, 18, 19, and 20; or (ii) a light chain variable region antibody sequence comprising a CDR1 sequence that is selected from the group consisting of SEQ ID NOs: 21, 22, 23, and 24, a CDR2 sequence that is selected from the group consisting of SEQ ID NOs: 25, 26, 27, and 28, and a CDR3 sequence that is selected from the group consisting of SEQ ID NOs: 29, 30, 31, and 32;

(b) altering at least one amino acid residue within at least one variable region antibody sequence, said sequence being selected from the heavy chain variable region antibody sequence and the light chain variable region antibody sequence, to create at least one altered antibody sequence; and

(c) expressing the altered antibody sequence as a protein.

In another aspect, the invention provides a method of inhibiting or preventing growth of a tumor (e.g., prostate, colon, renal, rectal, urothelial, breast, bladder, liver, pancreas or melanoma) in a subject, wherein cells of the tumor or vascular endothelial cells proximate to the tumor express PSMA. The method includes administering to a subject an anti-PSMA antibody, or antigen-binding portion thereof, in combination with an anti-tumor agent both in an amount effective to inhibit or prevent growth of the tumor.

In another aspect, the invention provides a method of stimulating antibody dependent cell-mediated cytotoxicity (ADCC) of a tumor in a subject, wherein cells of the tumor or vascular endothelial cells proximate to the tumor express PSMA. The method includes administering to a subject an anti-PSMA antibody, or antigen-binding portion thereof, in combination with an anti-tumor agent both in an amount effective to stimulate antibody dependent cell-mediated cytotoxicity (ADCC) of the tumor.

In a further aspect, the invention provides a method of inhibiting tumor-related cachexia in a subject, wherein cells of the tumor or vascular endothelial cells proximate to the tumor express PSMA. The method includes administering to a subject an anti-PSMA antibody, or antigen-binding portion thereof, in combination with an anti-tumor agent both in an amount effective to inhibit tumor-related cachexia in the subject.

In one embodiment of the invention, administration of the anti-PSMA antibody, or antigen-binding portion thereof, to a subject, in combination with the anti-tumor agent, leads to a synergistic effect on the inhibition of the growth of the tumor. In another embodiment, the anti-tumor agent causes damage in the tumor mass, thereby leading to a more effective antibody dependent cell-mediated cytotoxicity (ADCC) of the tumor.

In another embodiment, the anti-PSMA antibody may be the 7F12 1C3, 2A10, 2F5 or 2C6 antibody.

In one embodiment of the invention, the anti-tumor agent is a chemotherapeutic agent, such as Taxotere® (docetaxel). In another embodiment, the anti-tumor agent is an anti-angiogenic agent, such as angiostatin K1-3, Arresten, aaAT, Canstatin, DL-α-Difluoromethyl-ornithine, Endostatin, Fumagillin, Genistein, Minocycline, Staurosporine, Thalidomide, and Tumstatin. In another embodiment, the anti-tumor agent is an immunomodulatory agent, such as anti-PD1 antibodies, anti-CTLA-4 antibodies, phosphorothiolate oligodeoxyribonucleotide (1018 ISS), GM-CSF gene vaccines, interleukin-2, interleukin-7 (CYT 99 07), interleukin-12 and interleukin-21.

In another aspect, the invention provides method of identifying an anti-tumor agent capable of acting synergistically with an anti-PSMA antibody in inhibiting or preventing growth of a tumor, wherein cells of the tumor or vascular endothelial cells proximate to the tumor express PSMA. The method includes contacting an indicator composition with (a) a test anti-tumor agent alone, (b) an anti-PSMA antibody alone, and (c) both a test anti-tumor agent and an anti-PSMA antibody; and comparing the ability of (a) the test anti-tumor agent alone and (b) the anti-PSMA antibody alone to inhibit or prevent growth of a tumor to the ability of (c) both the test anti-tumor agent and the anti-PSMA antibody to inhibit or prevent growth of a tumor, wherein inhibition or prevention of tumor growth by (c) in an amount that is greater than the additive effect of (a) and (b) would lead to the identification of an anti-tumor agent capable of acting synergistically with an anti-PSMA antibody in inhibiting or preventing growth of a tumor.

In another aspect, the invention pertains to a composition comprising an anti-PSMA antibody (e.g., 7F12 1C3, 2A10, 2F5 or 2C6) and an anti-tumor agent (e.g., Taxotere® (docetaxel), angiostatin K1-3, Arresten, aaAT, Canstatin, DL-α-Difluoromethyl-ornithine, Endostatin, Fumagillin, Genistein, Minocycline, Staurosporine, Thalidomide, Tumstatin, an anti-PD1 antibody, an anti-CTLA-4 antibody, phosphorothiolate oligodeoxyribonucleotide (1018 ISS), a GM-CSF gene vaccine, interleukin-2, interleukin-7 (CYT 99 07), interleukin-12 or interleukin-21) in an amount effective to inhibit or prevent growth of a tumor and a pharmaceutically acceptable carrier or in an amount effective to stimulate antibody dependent cell-mediated cytotoxicity (ADCC) of a tumor and a pharmaceutically acceptable carrier.

Other features and advantages of the instant invention will be apparent from the following detailed description and examples which should not be construed as limiting. The contents of all references, Genbank entries, patents and published patent applications cited throughout this application are expressly incorporated herein by reference.

### BRIEF DESCRIPTION OF THE DRAWING

FIG. 1A shows the nucleotide sequence (SEQ ID NO: 33) and amino acid sequence (SEQ ID NO: 1) of the heavy chain variable region of the 1C3 human monoclonal antibody. The CDR1 (SEQ ID NO: 9), CDR2 (SEQ ID NO: 13) and CDR3 (SEQ ID NO: 17) regions are delineated and the V, D and J germline derivations are indicated.

FIG. 1B shows the nucleotide sequence (SEQ ID NO: 37) and amino acid sequence (SEQ ID NO: 5) of the light chain variable region of the 1C3 human monoclonal antibody. The CDR1 (SEQ ID NO: 21), CDR2 (SEQ ID NO: 25) and CDR3 (SEQ ID NO: 29) regions are delineated and the V and J germline derivations are indicated.

FIG. 2A shows the nucleotide sequence (SEQ ID NO: 34) and amino acid sequence (SEQ ID NO: 2) of the heavy chain variable region of the 2A10 human monoclonal antibody. The CDR1 (SEQ ID NO: 10), CDR2 (SEQ ID NO: 14) and CDR3 (SEQ ID NO: 18) regions are delineated and the V and J germline derivations are indicated.

FIG. 2B shows the nucleotide sequence (SEQ ID NO: 38) and amino acid sequence (SEQ ID NO: 6) of the light chain variable region of the 2A10 human monoclonal antibody. The CDR1 (SEQ ID NO: 22), CDR2 (SEQ ID NO: 26) and CDR3 (SEQ ID NO: 30) regions are delineated and the V and J germline derivations are indicated.

FIG. 3A shows the nucleotide sequence (SEQ ID NO: 35) and amino acid sequence (SEQ ID NO: 3) of the heavy chain variable region of the 2F5 human monoclonal antibody. The CDR1 (SEQ ID NO: 11), CDR2 (SEQ ID NO: 15) and CDR3 (SEQ ID NO: 19) regions are delineated and the V and J germline derivations are indicated.

FIG. 3B shows the nucleotide sequence (SEQ ID NO: 39) and amino acid sequence (SEQ ID NO: 7) of the light chain variable region of the 2F5 human monoclonal antibody. The CDR1 (SEQ ID NO: 23), CDR2 (SEQ ID NO: 27) and CDR3 (SEQ ID NO: 31) regions are delineated and the V and J germline derivations are indicated.

FIG. 4A shows the nucleotide sequence (SEQ ID NO: 36) and amino acid sequence (SEQ ID NO: 4) of the heavy chain variable region of the 2C6 human monoclonal antibody. The CDR1 (SEQ ID NO: 12), CDR2 (SEQ ID NO: 16) and CDR3 (SEQ ID NO: 20) regions are delineated and the V and J germline derivations are indicated.

FIG. 4B shows the nucleotide sequence (SEQ ID NO: 40) and amino acid sequence (SEQ ID NO: 8) of the light chain variable region of the 2C6 human monoclonal antibody. The CDR1 (SEQ ID NO: 24), CDR2 (SEQ ID NO: 28) and CDR3 (SEQ ID NO: 32) regions are delineated and the V and J germline derivations are indicated.

FIG. 5 shows the alignment of the amino acid sequence of the heavy chain variable region of 1C3 (SEQ ID NO: 1) with the human germline V_{H} 3-30.3 amino acid sequence (SEQ ID NO: 41) and the JH6b germline (SEQ ID NO: 45).

FIG. 6 shows the alignment of the amino acid sequence of the heavy chain variable region of 2A10 (SEQ ID NO: 2), 2F5 (SEQ ID NO: 3), and 2C6 (SEQ ID NO: 4) with the human germline V_{H} 5-51 amino acid sequence (SEQ ID NO: 42).

FIG. 7 shows the alignment of the amino acid sequence of the light chain variable region of 1C3 (SEQ ID NO: 5), 2A10 (SEQ ID NO: 6), and 2F5 (residues 1-107 of SEQ ID NO: 7) with the human germline Vₖ L18 amino acid sequence (SEQ ID NO:43) and the JK4 germline (SEQ ID NO: 46).

FIG. 8 shows the alignment of the amino acid sequence of the light chain variable region of 2C6 (SEQ ID NO: 8) with the human germline Vₖ L6 amino acid sequence (SEQ ID NO:44) and the JK3 germline (SEQ ID NO: 47).

FIG. 9 shows the results of flow cytometry experiments demonstrating that the human monoclonal antibodies 2F5, 2A10, and 2C6, directed against human PSMA, binds the cell surface of PSMA-expressing LNCaP cells.

FIG. 10 shows the results of ELISA experiments demonstrating that human monoclonal antibodies against human PSMA specifically bind to PSMA purified from LNCaP cells.

FIGS. 11A-11B are results of antibody binding competition studies that demonstrate that the human monoclonal antibodies 2A10 and 7F12, directed against human PSMA, compete for binding to PSMA-expressing LNCaP prostate cancer cells. FIG. 11 shows competition of ¹²⁵I-2A10 with cold 7F12 antibody. FIG. 11B shows competition of ¹²⁵I-7F12 with cold 2A10 antibody.

FIGS. 12A-12B shows the results of internalization experiments demonstrating that the human monoclonal antibody 2A10, directed against human PSMA, enters PSMA-expressing LNCaP prostate cancer cells by a ³H-thymidine release assay. FIG. 12A shows results for LNCaP cells seeded for 2 hours prior to introduction of antibody. FIG. 12B shows results for LNCaP cells seeded overnight prior to introduction of antibody.

FIGS. 13A-13B are graphs depicting the mean and median LNCaP tumor xenografts growth curves for mice treated with either: the anti-PSMA 7F12 antibody, the isotype control antibody Rituxan, Taxotere (2 mg/kg), Taxotere (4 mg/kg), the anti-PSMA 7F12 antibody in combination with Taxotere (2 mg/kg), the anti-PSMA 7F12 antibody in combination with Taxotere (4 mg/kg), the isotype control antibody Rituxan in combination with Taxotere (2 mg/kg), the isotype control antibody Rituxan in combination with Taxotere (4 mg/kg), or PBS. FIGS. 13C-13D are graphs depicting the mean and median body weight change of LNCaP tumor-bearing mice treated with either: the anti-PSMA 7F12 antibody, the isotype control antibody Rituxan, Taxotere (2 mg/kg), Taxotere (4 mg/kg), the anti-PSMA 7F12 antibody in combination with Taxotere (2 mg/kg), the anti-PSMA 7F12 antibody in combination with Taxotere (4 mg/kg), the isotype control antibody Rituxan in combination with Taxotere (2 mg/kg), the isotype control antibody Rituxan in combination with Taxotere (4 mg/kg), or PBS.

FIGS. 14A-14B are graphs depicting the mean and median LNCaP tumor xenografts growth curves for mice treated with either: the anti-PSMA 7F12 antibody, the isotype control antibody Rituxan, or PBS. FIGS. 14C-14D are graphs depicting the mean and median body weight change of LNCaP tumor-bearing mice treated with either: the anti-PSMA 7F12 antibody, the isotype control antibody Rituxan, or PBS.

FIGS. 15A-15B are graphs depicting the mean and median LNCaP tumor xenografts growth curves for mice treated with either: Taxotere (4 mg/kg), the isotype control antibody Rituxan in combination with Taxotere (4 mg/kg), the anti-PSMA 7F12 antibody in combination with Taxotere (4 mg/kg), or PBS. FIGS. 15C-15D are graphs depicting the mean and median body weight change of LNCaP tumor-bearing mice treated with either: Taxotere (4 mg/kg), the isotype control antibody Rituxan in combination with Taxotere (4 mg/kg), the anti-PSMA 7F12 antibody in combination with Taxotere (4 mg/kg), or PBS.

FIGS. 16A-16B are graphs depicting the mean and median LNCaP tumor xenografts growth curves for mice treated with either: Taxotere (2 mg/kg), the anti-PSMA 7F12 antibody in combination with Taxotere (2 mg/kg), the isotype control antibody Rituxan in combination with Taxotere (2 mg/kg), or PBS. FIGS. 16C-16D are graphs depicting the mean and median body weight change of LNCaP tumor-bearing mice treated with either: Taxotere (2 mg/kg), the anti-PSMA 7F12 antibody in combination with Taxotere (2 mg/kg), the isotype control antibody Rituxan in combination with Taxotere (2 mg/kg), or PBS.

FIG. 17A-B shows the results of a cell proliferation assay demonstrating that toxin-conjugated human monoclonal anti-PSMA antibodies show cytotoxicity to prostate cancer cells (A) with a three-hour wash and (B) with a continuous wash.

FIG. 18 is a graph of changes in tumor volume over time for mice dosed with an isotype control antibody-drug conjugate, a αPSMA antibody-drug conjugate, or a conjugation buffer alone (vehicle).

FIG. 19 is a graph of changes in tumor volume over time for mice dosed with various amounts of a αPSMA antibody-drug conjugate or a conjugation buffer alone (vehicle).

FIG. 20 is a graph of changes in tumor volume over time for mice dosed with various amounts of an isotype control antibody-drug conjugate or a conjugation buffer alone (vehicle).

FIG. 21 is a graph of body weight change over time for mice dosed with various amounts of an isotype control antibody-drug conjugate or a conjugation buffer alone (vehicle).

FIG. 22 is a graph of body weight change over time for mice dosed with various amounts of a αPSMA antibody-drug conjugate or a conjugation buffer alone (vehicle).

FIG. 23 is a graph of changes in tumor volume over time, for tumors having an initial average tumor volume of 240 mm^{,} for mice dosed with an isotype control antibody-drug conjugate, a αPSMA antibody-drug conjugate, or a conjugation buffer alone (vehicle).

FIG. 24 is a graph of changes in tumor volume over time, for tumors having an initial average tumor volume of 430 mm³, for mice dosed with a αPSMA antibody-drug conjugate or a conjugation buffer alone (vehicle).

### DETAILED DESCRIPTION OF THE INVENTION

The present invention relates to isolated monoclonal antibodies, particularly human monoclonal antibodies, that bind specifically to PSMA with high affinity. In certain embodiments, the antibodies of the invention are derived from particular heavy and light chain germline sequences and/or comprise particular structural features such as CDR regions comprising particular amino acid sequences. The invention provides isolated antibodies, methods of making such antibodies, immunoconjugates and bispecific molecules comprising such antibodies and pharmaceutical compositions containing the antibodies, immunoconjugates or bispecific molecules of the invention. The invention also relates to methods of using the antibodies, such as to treat diseases such as cancer.

In order that the present invention may be more readily understood, certain terms are first defined. Additional definitions are set forth throughout the detailed description.

The terms “prostate specific membrane antigen” and “PSMA” are used interchangeably herein, and include any variants, isoforms and species homologs of human PSMA that are naturally expressed by cells and that retain binding to the antibodies 1C3, 2A10, 2F5 or 2C6 described herein. The complete amino acid sequence of human PSMA protein has the Genbank accession number NP_{—}004467. The complete cDNA sequence encoding the human PSMA protein has the Genbank accession number NM_{—}004476.

A “signal transduction pathway” refers to the biochemical relationship between a variety of signal transduction molecules that play a role in the transmission of a signal from one portion of a cell to another portion of a cell. As used herein, the phrase “cell surface receptor” includes, for example, molecules and complexes of molecules capable of receiving a signal and the transmission of such a signal across the plasma membrane of a cell. An example of a “cell surface receptor” of the present invention is the PSMA receptor.

The term “antibody” as referred to herein includes whole antibodies and any antigen binding fragment (i.e., “antigen-binding portion”) or single chains thereof. An “antibody” refers to a glycoprotein comprising at least two heavy (H) chains and two light (L) chains inter-connected by disulfide bonds, or an antigen binding portion thereof. Each heavy chain is comprised of a heavy chain variable region (abbreviated herein as V_{H}) and a heavy chain constant region. The heavy chain constant region is comprised of three domains, C_{H1}, C_{H2}, and C_{H3}. Each light chain is comprised of a light chain variable region (abbreviated herein as V_{L}) and a light chain constant region. The light chain constant region is comprised of one domain, C_{L}. The V_{H} and V_{L} regions can be further subdivided into regions of hypervariability, termed complementarity determining regions (CDR), interspersed with regions that are more conserved, termed framework regions (FR). Each V_{H} and V_{L} is composed of three CDRs and four FRs, arranged from amino-terminus to carboxy-terminus in the following order: FR1, CDR1, FR2, CDR2, FR3, CDR3, FR4. The variable regions of the heavy and light chains contain a binding domain that interacts with an antigen. The constant regions of the antibodies may mediate the binding of the immunoglobulin to host tissues or factors, including various cells of the immune system (e.g., effector cells) and the first component (Clq) of the classical complement system.

The term “antigen-binding portion” of an antibody (or simply “antibody portion”), as used herein, refers to one or more fragments of an antibody that retain the ability to specifically bind to an antigen (e.g., PSMA). It has been shown that the antigen-binding function of an antibody can be performed by fragments of a full-length antibody. Examples of binding fragments encompassed within the term “antigen-binding portion” of an antibody include (i) a Fab fragment, a monovalent fragment consisting of the V_{L}, V_{H}, C_{L} and C_{H1} domains; (ii) a F(ab′)₂ fragment, a bivalent fragment comprising two Fab fragments linked by a disulfide bridge at the hinge region; (iii) a Fd fragment consisting of the V_{H} and C_{H1} domains; (iv) a Fv fragment consisting of the V_{L} and V_{H} domains of a single arm of an antibody, (v) a dAb fragment (Ward et al., (1989) Nature 341:544-546), which consists of a V_{H} domain; and (vi) an isolated complementarity determining region (CDR). Furthermore, although the two domains of the Fv fragment, V_{L} and V_{H}, are coded for by separate genes, they can be joined, using recombinant methods, by a synthetic linker that enables them to be made as a single protein chain in which the V_{L} and V_{H} regions pair to form monovalent molecules (known as single chain Fv (scFv); see e.g., Bird et al. (1988) Science 242:423-426; and Huston et al. (1988) Proc. Natl. Acad. Sci. USA 85:5879-5883). Such single chain antibodies are also intended to be encompassed within the term “antigen-binding portion” of an antibody. These antibody fragments are obtained using conventional techniques known to those with skill in the art, and the fragments are screened for utility in the same manner as are intact antibodies.

An “isolated antibody”, as used herein, is intended to refer to an antibody that is substantially free of other antibodies having different antigenic specificities (e.g. an isolated antibody that specifically binds PSMA is substantially free of antibodies that specifically bind antigens other than PSMA). An isolated antibody that specifically binds PSMA may, however, have cross-reactivity to other antigens, such as PSMA molecules from other species. Moreover, an isolated antibody may be substantially free of other cellular material and/or chemicals.

The terms “monoclonal antibody” or “monoclonal antibody composition” as used herein refer to a preparation of antibody molecules of single molecular composition. A monoclonal antibody composition displays a single binding specificity and affinity for a particular epitope.

The term “human antibody”, as used herein, is intended to include antibodies having variable regions in which both the framework and CDR regions are derived from human germline immunoglobulin sequences. Furthermore, if the antibody contains a constant region, the constant region also is derived from human germline immunoglobulin sequences. The human antibodies of the invention may include amino acid residues not encoded by human germline immunoglobulin sequences (e.g., mutations introduced by random or site-specific mutagenesis in vitro or by somatic mutation in vivo). However, the term “human antibody”, as used herein, is not intended to include antibodies in which CDR sequences derived from the germline of another mammalian species, such as a mouse, have been grafted onto human framework sequences.

The term “human monoclonal antibody” refers to antibodies displaying a single binding specificity which have variable regions in which both the framework and CDR regions are derived from human germline immunoglobulin sequences. In one embodiment, the human monoclonal antibodies are produced by a hybridoma which includes a B cell obtained from a transgenic nonhuman animal, e.g., a transgenic mouse, having a genome comprising a human heavy chain transgene and a light chain transgene fused to an immortalized cell.

The term “recombinant human antibody”, as used herein, includes all human antibodies that are prepared, expressed, created or isolated by recombinant means, such as (a) antibodies isolated from an animal (e.g., a mouse) that is transgenic or transchromosomal for human immunoglobulin genes or a hybridoma prepared therefrom (described further below), (b) antibodies isolated from a host cell transformed to express the human antibody, e.g., from a transfectoma, (c) antibodies isolated from a recombinant, combinatorial human antibody library, and (d) antibodies prepared, expressed, created or isolated by any other means that involve splicing of human immunoglobulin gene sequences to other DNA sequences. Such recombinant human antibodies have variable regions in which the framework and CDR regions are derived from human germline immunoglobulin sequences. In certain embodiments, however, such recombinant human antibodies can be subjected to in vitro mutagenesis (or, when an animal transgenic for human Ig sequences is used, in vivo somatic mutagenesis) and thus the amino acid sequences of the V_{H} and V_{L} regions of the recombinant antibodies are sequences that, while derived from and related to human germline V_{H} and V_{L} sequences, may not naturally exist within the human antibody germline repertoire in vivo.

As used herein, “isotype” refers to the antibody class (e.g., IgM or IgG1) that is encoded by the heavy chain constant region genes.

The phrases “an antibody recognizing an antigen” and “an antibody specific for an antigen” are used interchangeably herein with the term “an antibody which binds specifically to an antigen.”

The term “human antibody derivatives” refers to any modified form of the human antibody, e.g., a conjugate of the antibody and another agent or antibody.

The term “humanized antibody” is intended to refer to antibodies in which CDR sequences derived from the germline of another mammalian species, such as a mouse, have been grafted onto human framework sequences. Additional framework region modifications may be made within the human framework sequences.

The term “chimeric antibody” is intended to refer to antibodies in which the variable region sequences are derived from one species and the constant region sequences are derived from another species, such as an antibody in which the variable region sequences are derived from a mouse antibody and the constant region sequences are derived from a human antibody.

As used herein, an antibody that “specifically binds to human PSMA” is intended to refer to an antibody that binds to human PSMA with a K_{D} of 5×10⁻⁸ M or less, more preferably 1×10⁻⁸ M or less, more preferably 5×10⁻⁹ M or less, more preferably 1.2×10⁻⁹ M or less, even more preferably between 1.2×10⁻⁹ M and 1.2×10⁻¹⁰ M or less.

The term “K_{assoc}” or “Kₐ”, as used herein, is intended to refer to the association rate of a particular antibody-antigen interaction, whereas the term “K_{dis}” or “K_{d},” as used herein, is intended to refer to the dissociation rate of a particular antibody-antigen interaction. The term “K_{D}”, as used herein, is intended to refer to the dissociation constant, which is obtained from the ratio of K_{d} to Kₐ (i.e., K_{d}/Kₐ) and is expressed as a molar concentration (M). K_{D} values for antibodies can be determined using methods well established in the art. A preferred method for determining the K_{D} of an antibody is by using surface plasmon resonance, preferably using a biosensor system such as a Biacore® system.

As used herein, the term “high affinity” for an IgG antibody refers to an antibody having a K_{D} of 10⁻⁷ M or less, more preferably 10⁻⁸ M or less, more preferably 10⁻⁹ M or less, and even more preferably 10⁻¹⁰ M or less for a target antigen. However, “high affinity” binding can vary for other antibody isotypes. For example, “high affinity” binding for an IgM isotype refers to an antibody having a K_{D} of 10⁻⁷ M or less, more preferably 10⁻⁸ M or less, even more preferably 10⁻⁹ M or less. The term “vector,” as used herein, is intended to refer to a nucleic acid molecule capable of transporting another nucleic acid to which it has been linked. One type of vector is a “plasmid”, which refers to a circular double stranded DNA loop into which additional DNA segments may be ligated. Another type of vector is a viral vector, wherein additional DNA segments may be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) can be integrated into the genome of a host cell upon introduction into the host cell, and thereby are replicated along with the host genome. Moreover, certain vectors are capable of directing the expression of genes to which they are operatively linked. Such vectors are referred to herein as “recombinant expression vectors” (or simply, “expression vectors”). In general, expression vectors of utility in recombinant DNA techniques are often in the form of plasmids. In the present specification, “plasmid” and “vector” may be used interchangeably as the plasmid is the most commonly used form of vector. However, the invention is intended to include such other forms of expression vectors, such as viral vectors (e.g., replication defective retroviruses, adenoviruses and adeno-associated viruses), which serve equivalent functions.

The term “recombinant host cell” (or simply “host cell”), as used herein, is intended to refer to a cell into which a recombinant expression vector has been introduced. It should be understood that such terms are intended to refer not only to the particular subject cell but to the progeny of such a cell. Because certain modifications may occur in succeeding generations due to either mutation or environmental influences, such progeny may not, in fact, be identical to the parent cell, but are still included within the scope of the term “host cell” as used herein. Recombinant host cells include, for example, CHO cells, transfectomas, and lymphocytic cells.

As used herein, the term “subject” includes any human or nonhuman animal. The term “nonhuman animal” includes all vertebrates, e.g., mammals and non-mammals, such as nonhuman primates, sheep, dogs, cats, horses, cows, chickens, amphibians, reptiles, etc.

The terms “transgenic, nonhuman animal” refers to a nonhuman animal having a genome comprising one or more human heavy and/or light chain transgenes or transchromosomes (either integrated or non-integrated into the animal's natural genomic DNA) and which is capable of expressing fully human antibodies. For example, a transgenic mouse can have a human light chain transgene and either a human heavy chain transgene or human heavy chain transchromosome, such that the mouse produces human anti-PSMA antibodies when immunized with PSMA antigen and/or cells expressing PSMA. The human heavy chain transgene can be integrated into the chromosomal DNA of the mouse, as is the case for transgenic, e.g., HuMAb mice, or the human heavy chain transgene can be maintained extrachromosomally, as is the case for transchromosomal (e.g., KM) mice as described in WO 02/43478. Such transgenic and transchromosomal mice are capable of producing multiple isotypes of human monoclonal antibodies to PSMA (e.g., IgG, IgA and/or IgE) by undergoing V-D-J recombination and isotype switching.

As used herein, the term “subject” includes any human or nonhuman animal. The term “nonhuman animal” includes all vertebrates, e.g., mammals and non-mammals, such as nonhuman primates, sheep, dogs, cats, horses, cows chickens, amphibians, reptiles, etc.

Various aspects of the invention are described in further detail in the following subsections.

### Anti-PSMA Antibodies

The antibodies of the invention are characterized by particular functional features or properties of the antibodies. For example, the antibodies bind specifically to human PSMA. Preferably, an antibody of the invention binds to PSMA with high affinity, for example with a K_{D} of 5×10⁻⁷ M or less. As another example, the antibodies bind specifically to a PSMA-expressing LNCaP (ATCC CRL-1740) cell line. Standard assays to evaluate the binding ability of the antibodies toward PSMA are known in the art, including for example, ELISAs, Western blots and RIAs. Suitable assays are described in detail in the Examples. The binding kinetics (e.g., binding affinity) of the antibodies also can be assessed by standard assays known in the art, such as by ELISA, Scatchard, and Biacore analysis. Other preferred properties of the antibodies of the invention include the ability to be internalized by PSMA expressing cells and high thermostability. Internalization of antibodies can be assessed as described in Example 6. Thermostability can be assessed as described in Example 7. Preferred antibodies of the invention have a melting point of at least 65° C., more preferably, at least 66° C., even more preferably at least 67° C., even more preferably at least 68° C., even more preferably at least 69° C., even more preferably at least 70° C. and even more preferably at least 71° C. Preferably an antibody of the invention has melting point in a range of 67° C. to 72° C., more preferably 68° C. to 72° C., or 69° C. to 72° C., or 70° C. to 72° C. or 69° C. to 71.43° C., or the antibody has a melting point of approximately 71.43 C.

### Monoclonal Antibodies 1C3, 2A10, 2F5 and 2C6

Preferred antibodies of the invention are the human monoclonal antibodies 1C3, 2A10, 2F5, and 2C6, isolated and structurally characterized as described in Examples 1 and 2. The V_{H} amino acid sequences of 1C3, 2A10, 2F5, and 2C6 are shown in SEQ ID NOs: 1, 2, 3, and 4, respectively. The V_{L} amino acid sequences of 1C3, 2A10, 2F5, and 2C6 are shown in SEQ ID NOs: 5, 6, 7, and 8, respectively.

Given that each of these antibodies can bind to PSMA, the V_{H} and V_{L} sequences can be “mixed and matched” to create other anti-PSMA binding molecules of the invention. PSMA binding of such “mixed and matched” antibodies can be tested using the binding assays described above and in the Examples (e.g., FACS, or ELISAs). Preferably, when V_{H} and V_{L} chains are mixed and matched, a V_{H} sequence from a particular V_{H}/V_{L} pairing is replaced with a structurally similar V_{H} sequence. Likewise, preferably a V_{L} sequence from a particular V_{H}/V_{L} pairing is replaced with a structurally similar V_{L} sequence.

Accordingly, in one aspect, the invention provides an isolated monoclonal antibody, or antigen binding portion thereof comprising:

(a) a heavy chain variable region comprising an amino acid sequence selected from the group consisting of SEQ ID NOs: 1, 2, 3, and 4; and

(b) a light chain variable region comprising an amino acid sequence selected from the group consisting of SEQ ID NOs: 5, 6, 7, and 8;

wherein the antibody specifically binds PSMA, preferably human PSMA. Preferred heavy and light chain combinations include:

(a) a heavy chain variable region comprising the amino acid sequence of SEQ ID NO: 1; and (b) a light chain variable region comprising the amino acid sequence of SEQ ID NO: 5; or

(a) a heavy chain variable region comprising the amino acid sequence of SEQ ID NO: 2; and (b) a light chain variable region comprising the amino acid sequence of SEQ ID NO: 6; or

(a) a heavy chain variable region comprising the amino acid sequence of SEQ ID NO: 3; and (b) a light chain variable region comprising the amino acid sequence of SEQ ID NO: 7; or

(a) a heavy chain variable region comprising the amino acid sequence of SEQ ID NO: 4; and (b) a light chain variable region comprising the amino acid sequence of SEQ ID NO: 8.

In another aspect, the invention provides antibodies that comprise the heavy chain and light chain CDR1s, CDR2s and CDR3s of 1C3, 2A10, 2F5, and 2C6, or combinations thereof. The amino acid sequences of the V_{H} CDR1s of 1C3, 2A10, 2F5, and 2C6 are shown in SEQ ID NOs: 9, 10, 11, and 12, respectively. The amino acid sequences of the V_{H} CDR2s of 1C3, 2A10, 2F5, and 2C6 are shown in SEQ ID NOs: 13, 14, 15, and 16, respectively. The amino acid sequences of the V_{H} CDR3s of 1C3, 2A10, 2F5, and 2C6 are shown in SEQ ID NOs: 17, 18, 19, and 20, respectively. The amino acid sequences of the Vₖ CDR1s of 1C3, 2A10, 2F5, and 2C6 are shown in SEQ ID NOs: 21, 22, 23, and 24, respectively. The amino acid sequences of the Vₖ CDR2s of 1C3, 2A10, 2F5, and 2C6 are shown in SEQ ID NOs: 25, 26, 27, and 28, respectively. The amino acid sequences of the Vₖ CDR3s of 1C3, 2A10, 2F5, and 2C6 are shown in SEQ ID NOs: 29, 30, 31, and 32, respectively. The CDR regions are delineated using the Kabat system (Kabat, E. A., et al. (1991) Sequences of Proteins of Imnunological Interest, Fifth Edition, U.S. Department of Health and Human Services, NIH Publication No. 91-3242).

Given that each of these antibodies can bind to PSMA and that antigen-binding specificity is provided primarily by the CDR1, CDR2, and CDR3 regions, the V_{H} CDR1, CDR2, and CDR3 sequences and Vₖ CDR1, CDR2, and CDR3 sequences can be “mixed and matched” (i.e., CDRs from different antibodies can be mixed and match, although each antibody must contain a V_{H} CDR1, CDR2, and CDR3 and a Vₖ CDR1, CDR2, and CDR3) to create other anti-PSMA binding molecules of the invention. PSMA binding of such “mixed and matched” antibodies can be tested using the binding assays described above and in the Examples (e.g., FACS, ELISAs, Biacore analysis). Preferably, when V_{H} CDR sequences are mixed and matched, the CDR1, CDR2 and/or CDR3 sequence from a particular V_{H} sequence is replaced with a structurally similar CDR sequence(s). Likewise, when Vₖ CDR sequences are mixed and matched, the CDR1, CDR2 and/or CDR3 sequence from a particular Vₖ sequence preferably is replaced with a structurally similar CDR sequence(s). It will be readily apparent to the ordinarily skilled artisan that novel V_{H} and V_{L} sequences can be created by substituting one or more V_{H} and/or V_{L} CDR region sequences with structurally similar sequences from the CDR sequences disclosed herein for monoclonal antibodies antibodies 1C3, 2A10, 2F5, and 2C6.

Accordingly, in another aspect, the invention provides an isolated monoclonal antibody, or antigen binding portion thereof comprising:

(a) a heavy chain variable region CDR1 comprising an amino acid sequence selected from the group consisting of SEQ ID NOs: 9, 10, 11, and 12;

(b) a heavy chain variable region CDR2 comprising an amino acid sequence selected from the group consisting of SEQ ID NOs: 13, 14, 15, and 16;

(c) a heavy chain variable region CDR3 comprising an amino acid sequence selected from the group consisting of SEQ ID NOs: 17, 18, 19, and 20;

(d) a light chain variable region CDR1 comprising an amino acid sequence selected from the group consisting of SEQ ID NOs: 21, 22, 23, and 24;

(e) a light chain variable region CDR2 comprising an amino acid sequence selected from the group consisting of SEQ ID NOs: 25, 26, 27, and 28; and

(f) a light chain variable region CDR3 comprising an amino acid sequence selected from the group consisting of SEQ ID NOs: 29, 30, 31, and 32;

wherein the antibody specifically binds PSMA, preferably human PSMA.

In a preferred embodiment, the antibody comprises:

(a) a heavy chain variable region CDR1 comprising SEQ ID NO: 9;

(b) a heavy chain variable region CDR2 comprising SEQ ID NO: 13;

(c) a heavy chain variable region CDR3 comprising SEQ ID NO: 17;

(d) a light chain variable region CDR1 comprising SEQ ID NO: 21;

(e) a light chain variable region CDR2 comprising SEQ ID NO: 25; and

(f) a light chain variable region CDR3 comprising SEQ ID NO: 29.

In another preferred embodiment, the antibody comprises:

(a) a heavy chain variable region CDR1 comprising SEQ ID NO: 10;

(b) a heavy chain variable region CDR2 comprising SEQ ID NO: 14;

(c) a heavy chain variable region CDR3 comprising SEQ ID NO: 18;

(d) a light chain variable region CDR1 comprising SEQ ID NO: 22;

(e) a light chain variable region CDR2 comprising SEQ ID NO: 26; and

(f) a light chain variable region CDR3 comprising SEQ ID NO: 30.

In another preferred embodiment, the antibody comprises:

(a) a heavy chain variable region CDR1 comprising SEQ ID NO: 11;

(b) a heavy chain variable region CDR2 comprising SEQ ID NO: 15;

(c) a heavy chain variable region CDR3 comprising SEQ ID NO: 19;

(d) a light chain variable region CDR1 comprising SEQ ID NO: 23;

(e) a light chain variable region CDR2 comprising SEQ ID NO: 27; and

(f) a light chain variable region CDR3 comprising SEQ ID NO: 31.

In another preferred embodiment, the antibody comprises:

(a) a heavy chain variable region CDR1 comprising SEQ ID NO: 12;

(b) a heavy chain variable region CDR2 comprising SEQ ID NO: 16;

(c) a heavy chain variable region CDR3 comprising SEQ ID NO: 20;

(d) a light chain variable region CDR1 comprising SEQ ID NO: 24;

(e) a light chain variable region CDR2 comprising SEQ ID NO: 28; and

(f) a light chain variable region CDR3 comprising SEQ ID NO: 32.

### Antibodies Having Particular Germline Sequences

In certain embodiments, an antibody of the invention comprises a heavy chain variable region from a particular germline heavy chain immunoglobulin gene and/or a light chain variable region from a particular germline light chain immunoglobulin gene.

For example, in a preferred embodiment, the invention provides an isolated monoclonal antibody, or an antigen-binding portion thereof, comprising a heavy chain variable region that is the product of or derived from a human V_{H} 3-30.3 gene, wherein the antibody specifically binds PSMA. In another preferred embodiment, the invention provides an isolated monoclonal antibody, or an antigen-binding portion thereof, comprising a light chain variable region that is the product of or derived from a human V_{K} L18 gene, wherein the antibody specifically binds PSMA.

In yet another preferred embodiment, the invention provides an isolated monoclonal antibody, or antigen-binding portion thereof, wherein the antibody:

(a) comprises a heavy chain variable region that is the product of or derived from a human V_{H} 3-30.3 gene (SEQ ID NO: 41);

(b) comprises a light chain variable region that is the product of or derived from a human V_{K} L18 gene (SEQ ID NO: 43); and

wherein the antibody binds PSMA, preferably human PSMA.

In yet another preferred embodiment, the invention provides an isolated monoclonal antibody, or antigen-binding portion thereof, wherein the antibody:

(a) comprises a heavy chain variable region that is the product of or derived from a human V_{H} 5-51 gene (SEQ ID NO: 42);

(b) comprises a light chain variable region that is the product of or derived from a human V_{K} L18 gene (SEQ ID NO: 44); and

wherein the antibody binds PSMA, preferably human PSMA.

An example of an antibody having V_{H} and V_{K} of V_{H} 3-30.3 and V_{K} L18, respectively, is 1C3. Examples of antibodies having V_{H} and V_{K} of V_{H} 5-51 and V_{K} L18, respectively, are 2A10 and 2F5.

As used herein, a human antibody comprises heavy or light chain variable regions that is “the product of” or “derived from” a particular germline sequence if the variable regions of the antibody are obtained from a system that uses human germline immunoglobulin genes. Such systems include immunizing a transgenic mouse carrying human immunoglobulin genes with the antigen of interest or screening a human immunoglobulin gene library displayed on phage with the antigen of interest. A human antibody that is “the product of” or “derived from” a human germline immunoglobulin sequence can be identified as such by comparing the amino acid sequence of the human antibody to the amino acid sequences of human germline immunoglobulins and selecting the human germline immunoglobulin sequence that is closest in sequence (i.e., greatest % identity) to the sequence of the human antibody. A human antibody that is “the product of” or “derived from” a particular human germline immunoglobulin sequence may contain amino acid differences as compared to the germiline sequence, due to, for example, naturally-occurring somatic mutations or intentional introduction of site-directed mutation. However, a selected human antibody typically is at least 90% identical in amino acids sequence to an amino acid sequence encoded by a human germline immunoglobulin gene and contains amino acid residues that identify the human antibody as being human when compared to the germline immunoglobulin amino acid sequences of other species (e.g., murine germline sequences). In certain cases, a human antibody may be at least 95%, or even at least 96%, 97%, 98%, or 99% identical in amino acid sequence to the amino acid sequence encoded by the germline immunoglobulin gene. Typically, a human antibody derived from a particular human germline sequence Will display no more than 10 amino acid differences from the amino acid sequence encoded by the human germline immunoglobulin gene. In certain cases, the human antibody may display no more than 5, or even no more than 4, 3, 2, or 1 amino acid difference from the amino acid sequence encoded by the germline immunoglobulin gene.

### Homologous Antibodies

In yet another embodiment, an antibody of the invention comprises heavy and light chain variable regions comprising amino acid sequences that are homologous to the amino acid sequences of the preferred antibodies described herein, and wherein the antibodies retain the desired functional properties of the anti-PSMA antibodies of the invention.

For example, the invention provides an isolated monoclonal antibody, or antigen binding portion thereof, comprising a heavy chain variable region and a light chain variable region, wherein:

(a) the heavy chain variable region comprises an amino acid sequence that is at least 80% homologous to an amino acid sequence selected from the group consisting of SEQ ID NOs: 1, 2, 3, and 4;

(b) the light chain variable region comprises an amino acid sequence that is at least 80% homologous to an amino acid sequence selected from the group consisting of SEQ ID NOs: 5, 6, 7, and 8; and

(c) the antibody specifically binds to a PSMA-expressing LNCaP cell line.

In other embodiments, the V_{H} and/or V_{L} amino acid sequences may be 85%, 90%, 95%, 96%, 97%, 98% or 99% homologous to the sequences set forth above. An antibody having V_{H} and V_{L} regions having high (i.e., 80% or greater) homology to the V_{H} and V_{L} regions of the sequences set forth above, can be obtained by mutagenesis (e.g., site-directed or PCR-mediated mutagenesis) of nucleic acid molecules corresponding to SEQ ID NOs: 33, 34, 35, 36, 37, 38, 39, and 40, followed by testing of the encoded altered antibody for retained function (i.e., binding to human PSMA with a K_{D} of 5×10⁻⁸ M or less) using the functional assays described herein.

As used herein, the percent homology between two amino acid sequences is equivalent to the percent identity between the two sequences. The percent identity between the two sequences is a function of the number of identical positions shared by the sequences (i.e., % homology=# of identical positions/total # of positions x 100), taking into account the number of gaps, and the length of each gap, which need to be introduced for optimal alignment of the two sequences. The comparison of sequences and determination of percent identity between two sequences can be accomplished using a mathematical algorithm, as described in the non-limiting examples below.

The percent identity between two amino acid sequences can be determined using the algorithm of E. Meyers and W. Miller (Comput. Appl. Biosci., 4:11-17 (1988)) which has been incorporated into the ALIGN program (version 2.0), using a PAM120 weight residue table, a gap length penalty of 12 and a gap penalty of 4. In addition, the percent identity between two amino acid sequences can be determined using the Needleman and Wunsch (J. Mol. Biol. 48:444-453 (1970)) algorithm which has been incorporated into the GAP program in the GCG software package (available at http://www.gcg.com), using either a Blossum 62 matrix or a PAM250 matrix, and a gap weight of 16, 14, 12, 10, 8, 6, or 4 and a length weight of 1, 2, 3, 4, 5, or 6.

Additionally or alternatively, the protein sequences of the present invention can further be used as a “query sequence” to perform a search against public databases to, for example, identify related sequences. Such searches can be performed using the XBLAST program (version 2.0) of Altschul, et al. (1990) J. Mol. Biol. 215:403-10. BLAST protein searches can be performed with the XBLAST program, score=50, wordlength=3 to obtain amino acid sequences homologous to the antibody molecules of the invention. To obtain gapped alignments for comparison purposes, Gapped BLAST can be utilized as described in Altschul et al., (1997) Nucleic Acids Res. 25(17):3389-3402. When utilizing BLAST and Gapped BLAST programs, the default parameters of the respective programs (e.g., XBLAST and NBLAST) can be used. See http://www.ncbi.nlm.nih.gov.

Antibodies with Conservative Modifications

In certain embodiments, an antibody of the invention comprises a heavy chain variable region comprising CDR1, CDR2 and CDR3 sequences and a light chain variable region comprising CDR1, CDR2 and CDR3 sequences, wherein one or more of these CDR sequences comprise specified amino acid sequences based on the preferred antibodies described herein (e.g., 1C3, 2A10, 2F5, or 2C6), or conservative modifications thereof, and wherein the antibodies retain the desired functional properties of the anti-PSMA antibodies of the invention. Accordingly, the invention provides an isolated monoclonal antibody, or antigen binding portion thereof, comprising a heavy chain variable region comprising CDR1, CDR2, and CDR3 sequences and a light chain variable region comprising CDR1, CDR2, and CDR3 sequences, wherein:

(a) the heavy chain variable region CDR3 sequence comprises an amino acid sequence selected from the group consisting of amino acid sequences of SEQ ID NOs: 17, 18, 19, and 20, and conservative modifications thereof;

(b) the light chain variable region CDR3 sequence comprises an amino acid sequence selected from the group consisting of amino acid sequence of SEQ ID NOs: 29, 30, 31, and 32, and conservative modifications thereof; and

(c) the antibody specifically binds to a PSMA-expressing LNCaP cell line.

In a preferred embodiment, the heavy chain variable region CDR2 sequence comprises an amino acid sequence selected from the group consisting of amino acid sequences of SEQ ID NOs: 13, 14, 15, and 16, and conservative modifications thereof; and the light chain variable region CDR2 sequence comprises an amino acid sequence selected from the group consisting of amino acid sequences of SEQ ID NOs: 25, 26, 27, and 28, and conservative modifications thereof. In another preferred embodiment, the heavy chain variable region CDR1 sequence comprises an amino acid sequence selected from the group consisting of amino acid sequences of SEQ ID NOs: 9, 10, 11, and 12, and conservative modifications thereof; and the light chain variable region CDR1 sequence comprises an amino acid sequence selected from the group consisting of amino acid sequences of SEQ ID NOs: 21, 22, 23, and 24, and conservative modifications thereof.

As used herein, the term “conservative sequence modifications” is intended to refer to amino acid modifications that do not significantly affect or alter the binding characteristics of the antibody containing the amino acid sequence. Such conservative modifications include amino acid substitutions, additions and deletions. Modifications can be introduced into an antibody of the invention by standard techniques known in the art, such as site-directed mutagenesis and PCR-mediated mutagenesis. Conservative amino acid substitutions are ones in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine, tryptophan), nonpolar side chains (e.g., alanine, valine, leucine, isoleucine, proline, phenylalanine, methionine), beta-branched side chains (e.g., threonine, valine, isoleucine) and aromatic side chains (e.g., tyrosine, phenylalanine, tryptophan, histidine). Thus, one or more amino acid residues within the CDR regions of an antibody of the invention can be replaced with other amino acid residues from the same side chain family and the altered antibody can be tested for retained function (i.e., the function set forth in (c)) using the functional assays described herein.

Antibodies that Bind to the Same Epitope as Anti-PSMA Antibodies of the Invention

In another embodiment, the invention provides antibodies that bind to the same epitope on human PSMA as any of the PSMA monoclonal antibodies of the invention (i.e., antibodies that have the ability to cross-compete for binding to PSMA with any of the monoclonal antibodies of the invention). In preferred embodiments, the reference antibody for cross-competition studies can be the monoclonal antibody 1C3 (having V_{H} and V_{L} sequences as shown in SEQ ID NOs: 1 and 5, respectively), or the monoclonal antibody 2A10 (having V_{H} and V_{L} sequences as shown in SEQ ID NOs: 2 and 6, respectively), or the monoclonal antibody 2F5 (having V_{H} and V_{L} sequences as shown in SEQ ID NOs: 3 and 7, respectively), or the monoclonal antibody 2C6 (having V_{H} and V_{L} sequences as shown in SEQ ID NOs: 4 and 8, respectively). Such cross-competing antibodies can be identified based on their ability to cross-compete with 1C3, 2A10, 2F5, or 2C6 in standard PSMA binding assays. For example, BIAcore analysis, ELISA assays or flow cytometry may be used to demonstrate cross-competition with the antibodies of the current invention. The ability of a test antibody to inhibit the binding of, for example, 1C3, 2A10, 2F5, or 2C6, to human PSMA demonstrates that the test antibody can compete with 1C3, 2A10, 2F5, or 2C6 for binding to human PSMA and thus binds to the same epitope on human PSMA as 1C3, 2A10, 2F5, or 2C6. In a preferred embodiment, the antibody that binds to the same epitope on human PSMA as 1C3, 2A10, 2F5, or 2C6 is a human monoclonal antibody. Such human monoclonal antibodies can be prepared and isolated as described in the Examples.

### Engineered and Modified Antibodies

An antibody of the invention further can be prepared using an antibody having one or more of the V_{H} and/or V_{L} sequences disclosed herein as starting material to engineer a modified antibody, which modified antibody may have altered properties from the starting antibody. An antibody can be engineered by modifying one or more residues within one or both variable regions (i.e., V_{H} and/or V_{L}), for example within one or more CDR regions and/or within one or more framework regions. Additionally or alternatively, an antibody can be engineered by modifying residues within the constant region(s), for example to alter the effector function(s) of the antibody.

One type of variable region engineering that can be performed is CDR grafting. Antibodies interact with target antigens predominantly through amino acid residues that are located in the six heavy and light chain complementarity determining regions (CDRs). For this reason, the amino acid sequences within CDRs are more diverse between individual antibodies than sequences outside of CDRs. Because CDR sequences are responsible for most antibody-antigen interactions, it is possible to express recombinant antibodies that mimic the properties of specific naturally occurring antibodies by constructing expression vectors that include CDR sequences from the specific naturally occurring antibody grafted onto framework sequences from a different antibody with different properties (see, e.g., Riechmann, L. et al. (1998) Nature 332:323-327; Jones, P. et al. (1986) Nature 321:522-525; Queen, C. et al. (1989) Proc. Natl. Acad. See. U.S.A. 86:10029-10033; U.S. Pat. No. 5,225,539 to Winter, and U.S. Pat. Nos. 5,530,101; 5,585,089; 5,693,762 and 6,180,370 to Queen et al.)

Accordingly, another embodiment of the invention pertains to an isolated monoclonal antibody, or antigen binding portion thereof, comprising a heavy chain variable region comprising CDR1, CDR2, and CDR3 sequences comprising an amino acid sequence selected from the group consisting of SEQ ID NOs: 9, 10, 11, and 12, SEQ ID NOs: 13, 14, 15, and 16, and SEQ ID NOs: 17, 18, 19, and 20, respectively, and a light chain variable region comprising CDR1, CDR2, and CDR3 sequences comprising an amino acid sequence selected from the group consisting of SEQ ID NOs: 21, 22, 23, and 24, SEQ ID NOs: 25, 26, 27, and 28, and SEQ ID NOs: 29, 30, 31, and 32, respectively. Thus, such antibodies contain the V_{H} and V_{L} CDR sequences of monoclonal antibodies 1C3, 2A10, 2F5, or 2C6 yet may contain different framework sequences from these antibodies.

Such framework sequences can be obtained from public DNA databases or published references that include germline antibody gene sequences. For example, germline DNA sequences for human heavy and light chain variable region genes can be found in the “VBase” human germline sequence database (available on the Internet at www.mrc-cpe.cam.ac.uk/vbase), as well as in Kabat, E. A., et al. (1991) Sequences of Proteins of Immunological Interest, Fifth Edition, U.S. Department of Health and Human Services, NIH Publication No. 91-3242; Tomlinson, I. M., et al. (1992) “The Repertoire of Human Germline V_{H} Sequences Reveals about Fifty Groups of V_{H} Segments with Different Hypervariable Loops” J. Mol. Biol. 227:776-798; and Cox, J. P. L. et al. (1994) “A Directory of Human Germ-line V_{H} Segments Reveals a Strong Bias in their Usage” Eur. J. Immunol. 24:827-836; the contents of each of which are expressly incorporated herein by reference.

Preferred framework sequences for use in the antibodies of the invention are those that are structurally similar to the framework sequences used by selected antibodies of the invention, e.g., similar to the V_{H} 3-30.3 framework sequences (SEQ ID NO: 41) and/or the V_{H} 5-51 framework sequences (SEQ ID NO: 42) and/or the V_{K} L18 framework sequences (SEQ ID NO: 43) and/or the V_{K} L6 framework sequences (SEQ ID NO: 44) used by preferred monoclonal antibodies of the invention. The V_{H} CDR1, CDR2, and CDR3 sequences, and the V_{K} CDR1, CDR2, and CDR3 sequences, can be grafted onto framework regions that have the identical sequence as that found in the germline immunoglobulin gene from which the framework sequence derive, or the CDR sequences can be grafted onto framework regions that contain one or more mutations as compared to the germline sequences. For example, it has been found that in certain instances it is beneficial to mutate residues within the framework regions to maintain or enhance the antigen binding ability of the antibody (see e.g., U.S. Pat. Nos. 5,530,101; 5,585,089; 5,693,762 and 6,180,370 to Queen et al).

Another type of variable region modification is to mutate amino acid residues within the V_{H} and/or V_{K} CDR1, CDR2 and/or CDR3 regions to thereby improve one or more binding properties (e.g., affinity) of the antibody of interest. Site-directed mutagenesis or PCR-mediated mutagenesis can be performed to introduce the mutation(s) and the effect on antibody binding, or other functional property of interest, can be evaluated in in vitro or in vivo assays as described herein and provided in the Examples. Preferably conservative modifications (as discussed above) are introduced. The mutations may be amino acid substitutions, additions or deletions, but are preferably substitutions. Moreover, typically no more than one, two, three, four or five residues within a CDR region are altered.

Accordingly, in another embodiment, the invention provides isolated anti-PSMA monoclonal antibodies, or antigen binding portions thereof, comprising a heavy chain variable region comprising: (a) a V_{H} CDR1 region comprising an amino acid sequence selected from the group consisting of SEQ ID NOs: 9, 10, 11, and 12, or an amino acid sequence having one, two, three, four or five amino acid substitutions, deletions or additions as compared to SEQ ID NOs: 9, 10, 11, and 12; (b) a V_{H} CDR2 region comprising an amino acid sequence selected from the group consisting of SEQ ID NOs: 13, 14, 15, and 16, or an amino acid sequence having one, two, three, four or five amino acid substitutions, deletions or additions as compared to SEQ ID NOs: 13, 14, 15, and 16; (c) a V_{H} CDR3 region comprising an amino acid sequence selected from the group consisting of SEQ ID NOs: 17, 18, 19, and 20, or an amino acid sequence having one, two, three, four or five amino acid substitutions, deletions or additions as compared to SEQ ID NOs: 17, 18, 19, and 20; (d) a V_{K} CDR1 region comprising an amino acid sequence selected from the group consisting of SEQ ID NOs: 21, 22, 23, and 24, or an amino acid sequence having one, two, three, four or five amino acid substitutions, deletions or additions as compared to SEQ ID NOs: 21, 22, 23, and 24; (e) a V_{K} CDR2 region comprising an amino acid sequence selected from the group consisting of SEQ ID NOs: 25, 26, 27, and 28, or an amino acid sequence having one, two, three, four or five amino acid substitutions, deletions or additions as compared to SEQ ID NOs: 25, 26, 27, and 28; and (f) a V_{K} CDR3 region comprising an amino acid sequence selected from the group consisting of SEQ ID NOs: 29, 30, 31, and 32, or an amino acid sequence having one, two, three, four or five amino acid substitutions, deletions or additions as compared to SEQ ID NOs: 29, 30, 31, and 32.

Engineered antibodies of the invention include those in which modifications have been made to framework residues within V_{H} and/or V_{K}, e.g. to improve the properties of the antibody. Typically such framework modifications are made to decrease the immunogenicity of the antibody. For example, one approach is to “backmutate” one or more framework residues to the corresponding germline sequence. More specifically, an antibody that has undergone somatic mutation may contain framework residues that differ from the germline sequence from which the antibody is derived. Such residues can be identified by comparing the antibody framework sequences to the germline sequences from which the antibody is derived. Such “backmutated” antibodies are also intended to be encompassed by the invention. For example, for 2C6, amino acid residue #9 (within FR1) Of V_{H} is a serine whereas this residue in the corresponding V_{H} 5-51 germline sequence is an alanine. To return the framework region sequences to their germline configuration, the somatic mutations can be “backmutated” to the germline sequence by, for example, site-directed mutagenesis or PCR-mediated mutagenesis (e.g., residue 9 of FR1 of the V_{H} of 2C6 can be “backmutated” from serine to alanine).

As another example, for 2F5, amino acid residue #84 (within FR3) of V_{H} is an asparagine whereas this residue in the corresponding V_{H} 5-51 germline sequence is a serine. To return the framework region sequences to their germline configuration, for example, residue 18 of FR3 of the V_{H} of 2F5 can be “backmutated” from asparagine to serine.

Another type of framework modification involves mutating one or more residues within the framework region, or even within one or more CDR regions, to remove T cell epitopes to thereby reduce the potential immunogenicity of the antibody. This approach is also referred to as “deimmunization” and is described in further detail in U.S. Patent Publication No. 20030153043 by Carr et al.

In addition or alternative to modifications made within the framework or CDR regions, antibodies of the invention may be engineered to include modifications within the Fc region, typically to alter one or more functional properties of the antibody, such as serum half-life, complement fixation, Fc receptor binding, and/or antigen-dependent cellular cytotoxicity. Furthermore, an antibody of the invention may be chemically modified (e.g., one or more chemical moieties can be attached to the antibody) or be modified to alter its glycosylation, again to alter one or more functional properties of the antibody. Each of these embodiments is described in further detail below. The numbering of residues in the Fc region is that of the EU index of Kabat.

In one embodiment, the hinge region of CH1 is modified such that the number of cysteine residues in the hinge region is altered, e.g., increased or decreased. This approach is described further in U.S. Pat. No. 5,677,425 by Bodmer et al. The number of cysteine residues in the hinge region of CH1 is altered to, for example, facilitate assembly of the light and heavy chains or to increase or decrease the stability of the antibody.

In another embodiment, the Fc hinge region of an antibody is mutated to decrease the biological half life of the antibody. More specifically, one or more amino acid mutations are introduced into the CH2-CH3 domain interface region of the Fc-hinge fragment such that the antibody has impaired Staphylococcyl protein A (SpA) binding relative to native Fc-hinge domain SpA binding. This approach is described in further detail in U.S. Pat. No. 6,165,745 by Ward et al.

In another embodiment, the antibody is modified to increase its biological half life. Various approaches are possible. For example, one or more of the following mutations can be introduced: T252L, T254S, T256F, as described in U.S. Pat. No. 6,277,375 to Ward. Alternatively, to increase the biological half life, the antibody can be altered within the CH1 or CL region to contain a salvage receptor binding epitope taken from two loops of a CH2 domain of an Fc region of an IgG, as described in U.S. Pat. Nos. 5,869,046 and 6,121,022 by Presta et al.

In yet other embodiments, the Fc region is altered by replacing at least one amino acid residue with a different amino acid residue to alter the effector function(s) of the antibody. For example, one or more amino acids selected from amino acid residues 234, 235, 236, 237, 297, 318, 320 and 322 can be replaced with a different amino acid residue such that the antibody has an altered affinity for an effector ligand but retains the antigen-binding ability of the parent antibody. The effector ligand to which affinity is altered can be, for example, an Fc receptor or the C1 component of complement. This approach is described in further detail in U.S. Pat. Nos. 5,624,821 and 5,648,260, both by Winter et al.

In another example, one or more amino acids selected from amino acid residues 329, 331 and 322 can be replaced with a different amino acid residue such that the antibody has altered C1q binding and/or reduced or abolished complement dependent cytotoxicity (CDC). This approach is described in further detail in U.S. Pat. No. 6,194,551 by Idusogie et al.

In another example, one or more amino acid residues within amino acid positions 231 and 239 are altered to thereby alter the ability of the antibody to fix complement. This approach is described further in PCT Publication WO 94/29351 by Bodmer et al.

In yet another example, the Fc region is modified to increase the ability of the antibody to mediate antibody dependent cellular cytotoxicity (ADCC) and/or to increase the affinity of the antibody for an Fcγ receptor by modifying one or more amino acids at the following positions: 238, 239, 248, 249, 252, 254, 255, 256, 258, 265, 267, 268, 269, 270, 272, 276, 278, 280, 283, 285, 286, 289, 290, 292, 293, 294, 295, 296, 298, 301, 303, 305, 307, 309, 312, 315, 320, 322, 324, 326, 327, 329, 330, 331, 333, 334, 335, 337, 338, 340, 360, 373, 376, 378, 382, 388, 389, 398, 414, 416, 419, 430, 434, 435, 437, 438 or 439. This approach is described further in PCT Publication WO 00/42072 by Presta. Moreover, the binding sites on human IgG1 for FcγR1, FcγRII, FcγRIII and FcRn have been mapped and variants with improved binding have been described (see Shields, R. L. et al. (2001) J. Biol. Chem. 276:6591-6604). Specific mutations at positions 256, 290, 298, 333, 334 and 339 were shown to improve binding to FcγRIII. Additionally, the following combination mutants were shown to improve FcγRIII binding: T256A/S298A, S298A/E333A, S298A/K224A and S298A/E333A/K334A.

In still another embodiment, the glycosylation of an antibody is modified. For example, an aglycoslated antibody can be made (i.e., the antibody lacks glycosylation). Glycosylation can be altered to, for example, increase the affinity of the antibody for antigen. Such carbohydrate modifications can be accomplished by, for example, altering one or more sites of glycosylation within the antibody sequence. For example, one or more amino acid substitutions can be made that result in elimination of one or more variable region framework glycosylation sites to thereby eliminate glycosylation at that site. Such aglycosylation may increase the affinity of the antibody for antigen. Such an approach is described in further detail in U.S. Pat. Nos. 5,714,350 and 6,350,861 by Co et al.

Additionally or alternatively, an antibody can be made that has an altered type of glycosylation, such as a hypofucosylated antibody having reduced amounts of fucosyl residues or an antibody having increased bisecting GlcNac structures. Such altered glycosylation patterns have been demonstrated to increase the ADCC ability of antibodies. Such carbohydrate modifications can be accomplished by, for example, expressing the antibody in a host cell with altered glycosylation machinery. Cells with altered glycosylation machinery have been described in the art and can be used as host cells in which to express recombinant antibodies of the invention to thereby produce an antibody with altered glycosylation. For example, the cell lines Ms704, Ms705, and Ms709 lack the fucosyltransferase gene, FUT8 (alpha (1,6) fucosyltransferase), such that antibodies expressed in the Ms704, Ms705, and Ms709 cell lines lack fucose on their carbohydrates. The Ms704, Ms705, and Ms709 FUT8^{-/-} cell lines were created by the targeted disruption of the FUT8 gene in CHO/DG44 cells using two replacement vectors (see U.S. Patent Publication No. 20040110704 by Yamane et al. and Yamane-Ohnuki et al. (2004) Biotechnol Bioeng 87:614-22). As another example, EP 1,176,195 by Hanai et al. describes a cell line with a functionally disrupted FUT8 gene, which encodes a fucosyl transferase, such that antibodies expressed in such a cell line exhibit hypofucosylation by reducing or eliminating the alpha 1,6 bond-related enzyme. Hanai et al. also describe cell lines which have a low enzyme activity for adding fucose to the N-acetylglucosamine that binds to the Fc region of the antibody or does not have the enzyme activity, for example the rat myeloma cell line YB2/0 (ATCC CRL 1662). PCT Publication WO 03/035835 by Presta describes a variant CHO cell line, Lec13 cells, with reduced ability to attach fucose to Asn(297)-linked carbohydrates, also resulting in hypofucosylation of antibodies expressed in that host cell (see also Shields, R. L. et al. (2002) J. Biol. Chem. 277:26733-26740). PCT Publication WO 99/54342 by Umana et al. describes cell lines engineered to express glycoprotein-modifying glycosyl transferases (e.g., beta(1,4)—N-acetylglucosaminyltransferase III (GnTIII) such that antibodies expressed in the engineered cell lines exhibit increased bisecting GlcNac structures which results in increased ADCC activity of the antibodies (see also Umana et al. (1999) Nat. Biotech. 17:176-180). Alternatively, the fucose residues of the antibody may be cleaved off using a fucosidase enzyme. For example, the fucosidase alpha-L-fucosidase removes fucosyl residues from antibodies (Tarentino, A. L. et al. (1975) Biochem. 14:5516-23).

Another modification of the antibodies herein that is contemplated by the invention is pegylation. An antibody can be pegylated to, for example, increase the biological (e.g., serum) half life of the antibody. To pegylate an antibody, the antibody, or fragment thereof, typically is reacted with polyethylene glycol (PEG), such as a reactive ester or aldehyde derivative of PEG, under conditions in which one or more PEG groups become attached to the antibody or antibody fragment. Preferably, the pegylation is carried out via an acylation reaction or an alkylation reaction with a reactive PEG molecule (or an analogous reactive water-soluble polymer). As used herein, the term “polyethylene glycol” is intended to encompass any of the forms of PEG that have been used to derivatize other proteins, such as mono (C1-C10) alkoxy- or aryloxy-polyethylene glycol or polyethylene glycol-maleimide. In certain embodiments, the antibody to be pegylated is an aglycosylated antibody. Methods for pegylating proteins are known in the art and can be applied to the antibodies of the invention. See for example, EP 0 154 316 by Nishimura et al. and EP 0 401 384 by Ishikawa et al.

### Methods of Engineering Antibodies

As discussed above, the anti-PSMA antibodies having V_{H} and V_{K} sequences disclosed herein can be used to create new anti-PSMA antibodies by modifying the V_{H} and/or V_{K} sequences, or the constant region(s) attached thereto. Thus, in another aspect of the invention, the structural features of an anti-PSMA antibody of the invention, e.g. 1C3, 2A10, 2F5, or 2C6, are used to create structurally related anti-PSMA antibodies that retain at least one functional property of the antibodies of the invention, such as binding to human PSMA. For example, one or more CDR regions of 1C3, 2A10, 2F5, or 2C6, or mutations thereof, can be combined recombinantly with known framework regions and/or other CDRs to create additional, recombinantly-engineered, anti-PSMA antibodies of the invention, as discussed above. Other types of modifications include those described in the previous section. The starting material for the engineering method is one or more of the V_{H} and/or V_{K} sequences provided herein, or one or more CDR regions thereof. To create the engineered antibody, it is not necessary to actually prepare (i.e., express as a protein) an antibody having one or more of the V_{H} and/or V_{K} sequences provided herein, or one or more CDR regions thereof. Rather, the information contained in the sequence(s) is used as the starting material to create a “second generation” sequence(s) derived from the original sequence(s) and then the “second generation” sequence(s) is prepared and expressed as a protein.

Accordingly, in another embodiment, the invention provides a method for preparing an anti-PSMA antibody comprising:
(a) providing: (i) a heavy chain variable region antibody sequence comprising a CDR1 sequence selected from the group consisting of SEQ ID NOs: 9, 10, 11, and 12, a CDR2 sequence selected from the group consisting of SEQ ID NOs: 13, 14, 15, and 16, and/or a CDR3 sequence selected from the group consisting of SEQ ID NOs: 17, 18, 19, and 20; and/or (ii) a light chain variable region antibody sequence comprising a CDR1 sequence selected from the group consisting of SEQ ID NOs: 21, 22, 23, and 24, a CDR2 sequence selected from the group consisting of SEQ ID NOs: 25, 26, 27, and 28, and/or a CDR3 sequence selected from the group consisting of SEQ ID NOs: 29, 30, 31, and 32;
(b) altering at least one amino acid residue within the heavy chain variable region antibody sequence and/or the light chain variable region antibody sequence to create at least one altered antibody sequence; and
(c) expressing the altered antibody sequence as a protein.

Standard molecular biology techniques can be used to prepare and express the altered antibody sequence.
Preferably, the antibody encoded by the altered antibody sequence(s) is one that retains one, some or all of the functional properties of the anti-PSMA antibodies described herein, which functional properties include, but are not limited to specifically binding to a PSMA-expressing LNCaP cell line.

The functional properties of the altered antibodies can be assessed using standard assays available in the art and/or described herein, such as those set forth in the Examples (e.g., flow cytometry, binding assays).

In certain embodiments of the methods of engineering antibodies of the invention, mutations can be introduced randomly or selectively along all or part of an anti-PSMA antibody coding sequence and the resulting modified anti-PSMA antibodies can be screened for binding activity and/or other functional properties as described herein. Mutational methods have been described in the art. For example, PCT Publication WO 02/092780 by Short describes methods for creating and screening antibody mutations using saturation mutagenesis, synthetic ligation assembly, or a combination thereof. Alternatively, PCT Publication WO 03/074679 by Lazar et al. describes methods of using computational screening methods to optimize physiochemical properties of antibodies.

### Nucleic Acid Molecules Encoding Antibodies of the Invention

Another aspect of the invention pertains to nucleic acid molecules that encode the antibodies of the invention. The nucleic acids may be present in whole cells, in a cell lysate, or in a partially purified or substantially pure form. A nucleic acid is “isolated” or “rendered substantially pure” when purified away from other cellular components or other contaminants, e.g., other cellular nucleic acids or proteins, by standard techniques, including alkaline/SDS treatment, CsCl banding, column chromatography, agarose gel electrophoresis and others well known in the art. See, F. Ausubel, et al., ed. (1987) Current Protocols in Molecular Biology, Greene Publishing and Wiley Interscience, New York. A nucleic acid of the invention can be, for example, DNA or RNA and may or may not contain intronic sequences. In a preferred embodiment, the nucleic acid is a cDNA molecule.

Nucleic acids of the invention can be obtained using standard molecular biology techniques. For antibodies expressed by hybridomas (e.g., hybridomas prepared from transgenic mice carrying human immunoglobulin genes as described further below), cDNAs encoding the light and heavy chains of the antibody made by the hybridoma can be obtained by standard PCR amplification or cDNA cloning techniques. For antibodies obtained from an immunoglobulin gene library (e.g., using phage display techniques), nucleic acid encoding the antibody can be recovered from the library.

Preferred nucleic acids molecules of the invention are those encoding the VH and VL sequences of the 1C3, 2A10, 2F5, or 2C6 monoclonal antibodies. DNA sequences encoding the VH sequences of 1C3, 2A10, 2F5, and 2C6 are shown in SEQ ID NOs: 33, 34, 35, and 36, respectively. DNA sequences encoding the VL sequences of 1C3, 2A10, 2F5, and 2C6 are shown in SEQ ID NOs: 37, 38, 39, and 40, respectively.

Once DNA fragments encoding VH and VL segments are obtained, these DNA fragments can be further manipulated by standard recombinant DNA techniques, for example to convert the variable region genes to full-length antibody chain genes, to Fab fragment genes or to a scFv gene. In these manipulations, a VL- or VH-encoding DNA fragment is operatively linked to another DNA fragment encoding another protein, such as an antibody constant region or a flexible linker. The term “operatively linked”, as used in this context, is intended to mean that the two DNA fragments are joined such that the amino acid sequences encoded by the two DNA fragments remain in-frame.

The isolated DNA encoding the VH region can be converted to a full-length heavy chain gene by operatively linking the VH-encoding DNA to another DNA molecule encoding heavy chain constant regions (CH1, CH2 and CH3). The sequences of human heavy chain constant region genes are known in the art (see e.g., Kabat, E. A., et al. (1991) Sequences of Proteins of Immunological Interest, Fifth Edition, U.S. Department of Health and Human Services, NIH Publication No. 91-3242) and DNA fragments encompassing these regions can be obtained by standard PCR amplification. The heavy chain constant region can be an IgG1, IgG2, IgG3, IgG4, IgA, IgE, IgM or IgD constant region, but most preferably is an IgG1 or IgG4 constant region. For a Fab fragment heavy chain gene, the VH-encoding DNA can be operatively linked to another DNA molecule encoding only the heavy chain CH1 constant region.

The isolated DNA encoding the VL region can be converted to a full-length light chain gene (as well as a Fab light chain gene) by operatively linking the VL-encoding DNA to another DNA molecule encoding the light chain constant region, CL. The sequences of human light chain constant region genes are known in the art (see e.g., Kabat, E. A., et al. (1991) Sequences of Proteins of Immunological Interest, Fifth Edition, U.S. Department of Health and Human Services, NIH Publication No. 91-3242) and DNA fragments encompassing these regions can be obtained by standard PCR amplification. The light chain constant region can be a kappa or lambda constant region, but most preferably is a kappa constant region.

To create a scFv gene, the VH- and VL-encoding DNA fragments are operatively linked to another fragment encoding a flexible linker, e.g., encoding the amino acid sequence (Gly₄-Ser)₃ (SEQ ID NO: 48), such that the VH and VL sequences can be expressed as a contiguous single-chain protein, with the VL and VH regions joined by the flexible linker (see e.g., Bird et al. (1988) Science 242:423-426; Huston et al. (1988) Proc. Natl. Acad. Sci. USA 85:5879-5883; McCafferty et al., (1990) Nature 348:552-554).

### Production of Monoclonal Antibodies of the Invention

Monoclonal antibodies (mAbs) of the present invention can be produced by a variety of techniques, including conventional monoclonal antibody methodology e.g., the standard somatic cell hybridization technique of Kohler and Milstein (1975) Nature 256: 495. Although somatic cell hybridization procedures are preferred, in principle, other techniques for producing monoclonal antibody can be employed e.g., viral or oncogenic transformation of B lymphocytes.

The preferred animal system for preparing hybridomas is the murine system. Hybridoma production in the mouse is a very well-established procedure. Immunization protocols and techniques for isolation of immunized splenocytes for fusion are known in the art. Fusion partners (e.g., murine myeloma cells) and fusion procedures are also known.

Chimeric or humanized antibodies of the present invention can be prepared based on the sequence of a murine monoclonal antibody prepared as described above. DNA encoding the heavy and light chain immunoglobulins can be obtained from the murine hybridoma of interest and engineered to contain non-murine (e.g., human) immunoglobulin sequences using standard molecular biology techniques. For example, to create a chimeric antibody, the murine variable regions can be linked to human constant regions using methods known in the art (see e.g., U.S. Pat. No. 4,816,567 to Cabilly et al.). To create a humanized antibody, the murine CDR regions can be inserted into a human framework using methods known in the art (see e.g., U.S. Pat. No. 5,225,539 to Winter, and U.S. Pat. Nos. 5,530,101; 5,585,089; 5,693,762 and 6,180,370 to Queen et al.).

In a preferred embodiment, the antibodies of the invention are human monoclonal antibodies. Such human monoclonal antibodies directed against PSMA can be generated using transgenic or transchromosomic mice carrying parts of the human immune system rather than the mouse system. These transgenic and transchromosomic mice include mice referred to herein as HuMAb mice and KM Mice™, respectively, and are collectively referred to herein as “human Ig mice.”

The HuMAb mouses (Medarex, Inc.) contains human immunoglobulin gene miniloci that encode unrearranged human heavy (μ and γ) and κ light chain immunoglobulin sequences, together with targeted mutations that inactivate the endogenous μ and κ chain loci (see e.g., Lonberg, et al. (1994) Nature 368(6474): 856-859). Accordingly, the mice exhibit reduced expression of mouse IgM or κ, and in response to immunization, the introduced human heavy and light chain transgenes undergo class switching and somatic mutation to generate high affinity human IgGκ monoclonal (Lonberg, N. et al. (1994), supra; reviewed in Lonberg, N. (1994) Handbook of Experimental Pharmacology 113:49-101; Lonberg, N. and Huszar, D. (1995) Intern. Rev. Immunol. 13: 65-93, and Harding, F. and Lonberg, N. (1995) Ann. N.Y. Acad. Sci. 764:536-546). The preparation and use of HuMab mice, and the genomic modifications carried by such mice, is further described in Taylor, L. et al. (1992) Nucleic Acids Research 20:6287-6295; Chen, J. et al. (1993) International Immunology 5: 647-656; Tuaillon et al. (1993) Proc. Natl. Acad. Sci. USA 90:3720-3724; Choi et al. (1993) Nature Genetics 4:117-123; Chen, J. et al. (1993) EMBO J. 12: 821-830; Tuaillon et al. (1994) J. Immunol. 152:2912-2920; Taylor, L. et al. (1994) International Immunology 6: 579-591; and Fishwild, D. et al. (1996) Nature Biotechnology 14: 845-851, the contents of all of which are hereby specifically incorporated by reference in their entirety. See further, U.S. Pat. Nos. 5,545,806; 5,569,825; 5,625,126; 5,633,425; 5,789,650; 5,877,397; 5,661,016; 5,814,318; 5,874,299; and 5,770,429; all to Lonberg and Kay; U.S. Pat. No. 5,545,807 to Surani et al.; PCT Publication Nos. WO 92/03918, WO 93/12227, WO 94/25585, WO 97/13852, WO 98/24884 and WO 99/45962, all to Lonberg and Kay; and PCT Publication No. WO 01/14424 to Korman et al.

In another embodiment, human antibodies of the invention can be raised using a mouse that carries human immunoglobulin sequences on transgenes and transchomosomes, such as a mouse that carries a human heavy chain transgene and a human light chain transchromosome. Such mice, referred to herein as “KM Mice™”, are described in detail in PCT Publication WO 02/43478 to Ishida et al.

Still further, alternative transgenic animal systems expressing human immunoglobulin genes are available in the art and can be used to raise anti-PSMA antibodies of the invention. For example, an alternative transgenic system referred to as the Xenomouse (Abgenix, Inc.) can be used; such mice are described in, for example, U.S. Pat. Nos. 5,939,598; 6,075,181; 6,114,598; 6, 150,584 and 6,162,963 to Kucherlapati et al.

Moreover, alternative transchromosomic animal systems expressing human immunoglobulin genes are available in the art and can be used to raise anti-PSMA antibodies of the invention. For example, mice carrying both a human heavy chain transchromosome and a human light chain tranchromosome, referred to as “TC mice” can be used; such mice are described in Tomizuka et al. (2000) Proc. Natl. Acad. Sci. USA 97:722-727. Furthermore, cows carrying human heavy and light chain transchromosomes have been described in the art (Kuroiwa et al. (2002) Nature Biotechnology 20:889-894) and can be used to raise anti-PSMA antibodies of the invention.

Human monoclonal antibodies of the invention can also be prepared using phage display methods for screening libraries of human immunoglobulin genes. Such phage display methods for isolating human antibodies are established in the art. See for example: U.S. Pat. Nos. 5,223,409; 5,403,484; and 5,571,698 to Ladner et al.; U.S. Pat. Nos. 5,427,908 and 5,580,717 to Dower et al.; U.S. Pat. Nos. 5,969,108 and 6,172,197 to McCafferty et al.; and U.S. Pat. Nos. 5,885,793; 6,521,404; 6,544,731; 6,555,313; 6,582,915 and 6,593,081 to Griffiths et al.

Human monoclonal antibodies of the invention can also be prepared using SCID mice into which human immune cells have been reconstituted such that a human antibody response can be generated upon immunization. Such mice are described in, for example, U.S. Pat. Nos. 5,476,996 and 5,698,767 to Wilson et al.

### Immunization of Human Ig Mice

When human Ig mice are used to raise human antibodies of the invention, such mice can be immunized with a PSMA-expressing cell line, such as the LNCaP cell line (ATCC CRL-1740), a purified or enriched preparation of PSMA antigen and/or recombinant PSMA, or an PSMA fusion protein, as described by Lonberg, N. et al. (1994) Nature 368(6474): 856-859; Fishwild, D. et al. (1996) Nature Biotechnology 14: 845-851; and PCT Publication WO 98/24884 and WO 01/14424. Preferably, the mice will be 6-16 weeks of age upon the first infusion. For example, a purified or recombinant preparation (5-50 μg) of PSMA antigen can be used to immunize the human Ig mice intraperitoneally.

Detailed procedures to generate fully human monoclonal antibodies to PSMA are described in Example 1 below. Cumulative experience with various antigens has shown that the transgenic mice respond when initially immunized intraperitoneally (IP) with antigen in complete Freund's adjuvant, followed by every other week IP immunizations up to a total of 6) with antigen in incomplete Freund's adjuvant. However, adjuvants other than Freund's are also found to be effective. In addition, whole cells in the absence of adjuvant are found to be highly immunogenic. The immune response can be monitored over the course of the immunization protocol with plasma samples being obtained by retroorbital bleeds. The plasma can be screened by ELISA (as described below), and mice with sufficient titers of anti-PSMA human immunoglobulin can be used for fusions. Mice can be boosted intravenously with antigen 3 days before sacrifice and removal of the spleen. It is expected that 2-3 fusions for each immunization may need to be performed. Between 6 and 24 mice are typically immunized for each antigen. Usually both HCo7 and HCo12 strains are used. In addition, both HCo7 and HCo12 transgene can be bred together into a single mouse having two different human heavy chain transgenes (HCo7/HCo12). Alternatively or additionally, the KM Mouse™ strain can be used.

### Generation of Hybridomas Producing Human Monoclonal Antibodies of the Invention

To generate hybridomas producing human monoclonal antibodies of the invention, splenocytes and/or lymph node cells from immunized mice can be isolated and fused to an appropriate immortalized cell line, such as a mouse myeloma cell line. The resulting hybridomas can be screened for the production of antigen-specific antibodies. For example, single cell suspensions of splenic lymphocytes from immunized mice can be fused to one-sixth the number of P3X63-Ag8.653 nonsecreting mouse myeloma cells (ATCC, CRL 1580) with 50% PEG. Cells are plated at approximately 2×10⁵ in flat bottom microtiter plate, followed by a two week incubation in selective medium containing 20% fetal Clone Serum, 18% “653” conditioned media, 5% origen (IGEN), 4 mM L-glutamine, 1 mM sodium pyruvate, 5 mM HEPES, 0.055 mM 2-mercaptoethanol, 50 units/ml penicillin, 50 mg/ml streptomycin, 50 mg/ml gentamycin and 1×HAT (Sigma; the HAT is added 24 hours after the fusion). After approximately two weeks, cells can be cultured in medium in which the HAT is replaced with HT. Individual wells can then be screened by ELISA for human monoclonal IgM and IgG antibodies. Once extensive hybridoma growth occurs, medium can be observed usually after 10-14 days. The antibody secreting hybridomas can be replated, screened again, and if still positive for human IgG, the monoclonal antibodies can be subcloned at least twice by limiting dilution. The stable subclones can then be cultured in vitro to generate small amounts of antibody in tissue culture medium for characterization.

To purify human monoclonal antibodies, selected hybridomas can be grown in two-liter spinner-flasks for monoclonal antibody purification. Supernatants can be filtered and concentrated before affinity chromatography with protein A-sepharose (Pharmacia, Piscataway, N.J.). Eluted IgG can be checked by gel electrophoresis and high performance liquid chromatography to ensure purity. The buffer solution can be exchanged into PBS, and the concentration can be determined by OD280 using 1.43 extinction coefficient. The monoclonal antibodies can be aliquoted and stored at −80° C.

### Generation of Transfectomas Producing Monoclonal Antibodies of the Invention

Antibodies of the invention also can be produced in a host cell transfectoma using, for example, a combination of recombinant DNA techniques and gene transfection methods as is well known in the art (e.g., Morrison, S. (1985) Science 229:1202).

For example, to express the antibodies, or antibody fragments thereof, DNAs encoding partial or full-length light and heavy chains, can be obtained by standard molecular biology techniques (e.g., PCR amplification or cDNA cloning using a hybridoma that expresses the antibody of interest) and the DNAs can be inserted into expression vectors such that the genes are operatively linked to transcriptional and translational control sequences. In this context, the term “operatively linked” is intended to mean that an antibody gene is ligated into a vector such that transcriptional and translational control sequences within the vector serve their intended function of regulating the transcription and translation of the antibody gene. The expression vector and expression control sequences are chosen to be compatible with the expression host cell used. The antibody light chain gene and the antibody heavy chain gene can be inserted into separate vector or, more typically, both genes are inserted into the same expression vector. The antibody genes are inserted into the expression vector by standard methods (e.g., ligation of complementary restriction sites on the antibody gene fragment and vector, or blunt end ligation if no restriction sites are present). The light and heavy chain variable regions of the antibodies described herein can be used to create full-length antibody genes of any antibody isotype by inserting them into expression vectors already encoding heavy chain constant and light chain constant regions of the desired isotype such that the V_{H} segment is operatively linked to the CH segment(s) within the vector and the V_{K} segment is operatively linked to the CL segment within the vector. Additionally or alternatively, the recombinant expression vector can encode a signal peptide that facilitates secretion of the antibody chain from a host cell. The antibody chain gene can be cloned into the vector such that the signal peptide is linked in-frame to the amino terminus of the antibody chain gene. The signal peptide can be an immunoglobulin signal peptide or a heterologous signal peptide (i.e., a signal peptide from a non-immunoglobulin protein).

In addition to the antibody chain genes, the recombinant expression vectors of the invention carry regulatory sequences that control the expression of the antibody chain genes in a host cell. The term “regulatory sequence” is intended to include promoters, enhancers and other expression control elements (e.g., polyadenylation signals) that control the transcription or translation of the antibody chain genes. Such regulatory sequences are described, for example, in Goeddel (Gene Expression Technology. Methods in Enzymology 185, Academic Press, San Diego, Calif. (1990)). It will be appreciated by those skilled in the art that the design of the expression vector, including the selection of regulatory sequences, may depend on such factors as the choice of the host cell to be transformed, the level of expression of protein desired, etc. Preferred regulatory sequences for mammalian host cell expression include viral elements that direct high levels of protein expression in mammalian cells, such as promoters and/or enhancers derived from cytomegalovirus (CMV), Simian Virus 40 (SV40), adenovirus, (e.g., the adenovirus major late promoter (AdMLP) and polyoma. Alternatively, nonviral regulatory sequences may be used, such as the ubiquitin promoter or β-globin promoter. Still further, regulatory elements composed of sequences from different sources, such as the SRα promoter system, which contains sequences from the SV40 early promoter and the long terminal repeat of human T cell leukemia virus type 1 (Takebe, Y. et al. (1988) Mol. Cell. Biol. 8:466-472).

In addition to the antibody chain genes and regulatory sequences, the recombinant expression vectors of the invention may carry additional sequences, such as sequences that regulate replication of the vector in host cells (e.g., origins of replication) and selectable marker genes. The selectable marker gene facilitates selection of host cells into which the vector has been introduced (see, e.g., U.S. Pat. Nos. 4,399,216, 4,634,665 and 5,179,017, all by Axel et al.). For example, typically the selectable marker gene confers resistance to drugs, such as G418, hygromycin or methotrexate, on a host cell into which the vector has been introduced. Preferred selectable marker genes include the dihydrofolate reductase (DHFR) gene (for use in dhfr-host cells with methotrexate selection/amplification) and the neo gene (for G418 selection).

For expression of the light and heavy chains, the expression vector(s) encoding the heavy and light chains is transfected into a host cell by standard techniques. The various forms of the term “transfection” are intended to encompass a wide variety of techniques commonly used for the introduction of exogenous DNA into a prokaryotic or eukaryotic host cell, e.g., electroporation, calcium-phosphate precipitation, DEAE-dextran transfection and the like. Although it is theoretically possible to express the antibodies of the invention in either prokaryotic or eukaryotic host cells, expression of antibodies in eukaryotic cells, and most preferably mammalian host cells, is the most preferred because such eukaryotic cells, and in particular mammalian cells, are more likely than prokaryotic cells to assemble and secrete a properly folded and immunologically active antibody. Prokaryotic expression of antibody genes has been reported to be ineffective for production of high yields of active antibody (Boss, M. A. and Wood, C. R. (1985) Immunology Today 6:12-13).

Preferred mammalian host cells for expressing the recombinant antibodies of the invention include Chinese Hamster Ovary (CHO cells) (including dhfr-CHO cells, described in Urlaub and Chasin, (1980) Proc. Natl. Acad. Sci. USA 77:4216-4220, used with a DHFR selectable marker, e.g., as described in R. J. Kaufman and P. A. Sharp (1982) Mol. Biol. 159:601-621), NSO myeloma cells, COS cells and SP2 cells. In particular, for use with NSO myeloma cells, another preferred expression system is the GS gene expression system disclosed in WO 87/04462, WO 89/01036 and EP 338,841. When recombinant expression vectors encoding antibody genes are introduced into mammalian host cells, the antibodies are produced by culturing the host cells for a period of time sufficient to allow for expression of the antibody in the host cells or, more preferably, secretion of the antibody into the culture medium in which the host cells are grown. Antibodies can be recovered from the culture medium using standard protein purification methods.

### Characterization of Antibody Binding to Antigen

Antibodies of the invention can be tested for binding to PSMA by, for example, flow cytometry. Briefly, LNCaP cells are freshly harvested from tissue culture flasks and a single cell suspension prepared. LNCaP cell suspensions are either stained with primary antibody directly or after fixation with 1% paraformaldehyde in PBS. Approximately one million cells are resuspended in PBS containing 0.5% BSA and 50-200 μg/ml of primary antibody and incubated on ice for 30 minutes. The cells are washed twice with PBS containing 0.1% BSA, 0.01% NaN₃, resuspended in 100 μl of 1:100 diluted FITC-conjugated goat-anti-human IgG (Jackson ImmunoResearch, West Grove, Pa.), and incubated on ice for an additional 30 minutes. The cells are again washed twice, resuspended in 0.5 ml of wash buffer and analyzed for fluorescent staining on a FACSCalibur cytometer (Becton-Dickinson, San Jose, Calif.).

Alternatively, antibodies of the invention can be tested for binding to PSMA by standard ELISA. Briefly, microtiter plates are coated with purified PSMA at 0.25 μg/ml in PBS, and then blocked with 5% bovine serum albumin in PBS. Dilutions of antibody (e.g., dilutions of plasma from PSMA-immunized mice) are added to each well and incubated for 1-2 hours at 37° C. The plates are washed with PBS/Tween and then incubated with secondary reagent (e.g. for human antibodies, a goat-anti-human IgG Fc-specific polyclonal reagent) conjugated to alkaline phosphatase for 1 hour at 37° C. After washing, the plates are developed with pNPP substrate (1 mg/ml), and analyzed at OD of 405-650. Preferably, mice which develop the highest titers will be used for fusions.

An ELISA assay as described above can also be used to screen for hybridomas that show positive reactivity with PSMA immunogen. Hybridomas that bind with high avidity to PSMA are subcloned and further characterized. One clone from each hybridoma, which retains the reactivity of the parent cells (by ELISA), can be chosen for making a 5-10 vial cell bank stored at −140° C., and for antibody purification.

To purify anti-PSMA antibodies, selected hybridomas can be grown in two-liter spinner-flasks for monoclonal antibody purification. Supernatants can be filtered and concentrated before affinity chromatography with protein A-sepharose (Pharmacia, Piscataway, N.J.). Eluted IgG can be checked by gel electrophoresis and high performance liquid chromatography to ensure purity. The buffer solution can be exchanged into PBS, and the concentration can be determined by OD₂₈₀ using 1.43 extinction coefficient. The monoclonal antibodies can be aliquoted and stored at −80° C.

To determine if the selected anti-PSMA monoclonal antibodies bind to unique epitopes, each antibody can be biotinylated using commercially available reagents (Pierce, Rockford, Ill.). Competition studies using unlabeled monoclonal antibodies and biotinylated monoclonal antibodies can be performed using PSMA coated-ELISA plates as described above. Biotinylated mAb binding can be detected with a strep-avidin-alkaline phosphatase probe. Alternatively, competition studies can be performed using radiolabelled antibody and unlabelled competing antibodies can be detected in a Scatchard analysis, as further described in the Examples below.

To determine the isotype of purified antibodies, isotype ELISAs can be performed using reagents specific for antibodies of a particular isotype. For example, to determine the isotype of a human monoclonal antibody, wells of microtiter plates can be coated with 1 μg/ml of anti-human immunoglobulin overnight at 4° C. After blocking with 1% BSA, the plates are reacted with 1 μg/ml or less of test monoclonal antibodies or purified isotype controls, at ambient temperature for one to two hours. The wells can then be reacted with either human IgG1 or human IgM-specific alkaline phosphatase-conjugated probes. Plates are developed and analyzed as described above.

Anti-PSMA human IgGs can be further tested for reactivity with PSMA antigen by Western blotting. Briefly, PSMA can be prepared and subjected to sodium dodecyl sulfate polyacrylamide gel electrophoresis. After electrophoresis, the separated antigens are transferred to nitrocellulose membranes, blocked with 10% fetal calf serum, and probed with the monoclonal antibodies to be tested. Human IgG binding can be detected using anti-human IgG alkaline phosphatase and developed with BCIP/NBT substrate tablets (Sigma Chem. Co., St. Louis, Mo.).

### Immunoconjugates

In another aspect, the present invention features an anti-PSMA antibody, or a fragment thereof, conjugated to a therapeutic moiety, such as a cytotoxin, a drug (e.g., an immunosuppressant) or a radiotoxin. Such conjugates are referred to herein as “immunoconjugates”. Immunoconjugates that include one or more cytotoxins are referred to as “immunotoxins.” A cytotoxin or cytotoxic agent includes any agent that is detrimental to (e.g., kills) cells. Examples include taxol, cytochalasin B, gramicidin D, ethidium bromide, emetine, mitomycin, etoposide, tenoposide, vincristine, vinblastine, colchicin, doxorubicin, daunorubicin, dihydroxy anthracin dione, mitoxantrone, mithramycin, actinomycin D, 1-dehydrotestosterone, glucocorticoids, procaine, tetracaine, lidocaine, propranolol, and puromycin and analogs or homologs thereof. Therapeutic agents also include, for example, antimetabolites (e.g., methotrexate, 6-mercaptopurine, 6-thioguanine, cytarabine, 5-fluorouracil decarbazine), alkylating agents (e.g., mechlorethamine, thioepa chlorambucil, melphalan, carmustine (BSNU) and lomustine (CCNU), cyclothosphamide, busulfan, dibromomannitol, streptozotocin, mitomycin C, and cis-dichlorodiamine platinum (II) (DDP) cisplatin), anthracyclines (e.g., daunorubicin (formerly daunomycin) and doxorubicin), antibiotics (e.g., dactinomycin (formerly actinomycin), bleomycin, mithramycin, and anthramycin (AMC)), and anti-mitotic agents (e.g. vincristine and vinblastine).

Other preferred examples of therapeutic cytotoxins that can be conjugated to an antibody of the invention include duocarmycins, calicheamicins, maytansines and auristatins, and derivatives thereof. An example of a calicheamicin antibody conjugate is commercially available (Mylotarg™; Wyeth-Ayerst).

Cytoxins can be conjugated to antibodies of the invention using linker technology available in the art. Examples of linker types that have been used to conjugate a cytotoxin to an antibody include, but are not limited to, hydrazones, thioethers, esters, disulfides and peptide-containing linkers. A linker can be chosen that is, for example, susceptible to cleavage by low pH within the lysosomal compartment or susceptible to cleavage by proteases, such as proteases preferentially expressed in tumor tissue such as cathepsins (e.g., cathepsins B, C, D).

For further discussion of types of cytotoxins, linkers and methods for conjugating therapeutic agents to antibodies, see also Saito, G. et al. (2003) Adv. Drug Deliv. Rev. 55:199-215; Trail, P. A. et al. (2003) Cancer Immunol. Immunother. 52:328-337; Payne, G. (2003) Cancer Cell 3:207-212; Allen, T. M. (2002) Nat. Rev. Cancer 2:750-763; Pastan, L. and Kreitman, R. J. (2002) Curr. Opin. Investig. Drugs 3:1089-1091; Senter, P. D. and Springer, C. J. (2001) Adv. Drug Deliv. Rev. 53:247-264.

Antibodies of the present invention also can be conjugated to a radioactive isotope to generate cytotoxic radiopharmaceuticals, also referred to as radioimmunoconjugates. Examples of radioactive isotopes that can be conjugated to antibodies for use diagnostically or therapeutically include, but are not limited to, iodine¹³¹, iodine¹²⁵, indium¹¹¹, yttrium⁹⁰ and lutetium¹⁷⁷. Method for preparing radioimmunconjugates are established in the art. Examples of radioimmunoconjugates are commercially available, including Zevalin™ (IDEC Pharmaceuticals) and Bexxar™ (Corixa Pharmaceuticals), and similar methods can be used to prepare radioimmunoconjugates using the antibodies of the invention.

The antibody conjugates of the invention can be used to modify a given biological response, and the drug moiety is not to be construed as limited to classical chemical therapeutic agents. For example, the drug moiety may be a protein or polypeptide possessing a desired biological activity. Such proteins may include, for example, an enzymatically active toxin, or active fragment thereof, such as abrin, ricin A, pseudomonas exotoxin, or diphtheria toxin; a protein such as tumor necrosis factor or interferon-γ; or, biological response modifiers such as, for example, lymphokines, interleukin-1 (“IL-1”), interleukin-2 (“IL-2”), interleukin-6 (“IL-6”), granulocyte macrophage colony stimulating factor (“GM-CSF”), granulocyte colony stimulating factor (“G-CSF”), or other growth factors.

Techniques for conjugating such therapeutic moiety to antibodies are well known, see, e.g., Amon et al., “Monoclonal Antibodies For Immunotargeting Of Drugs In Cancer Therapy”, in Monoclonal Antibodies And Cancer Therapy, Reisfeld et al. (eds.), pp. 243-56 (Alan R. Liss, Inc. 1985); Hellstrom et al., “Antibodies For Drug Delivery”, in Controlled Drug Delivery (2nd Ed.), Robinson et al. (eds.), pp. 623-53 (Marcel Dekker, Inc. 1987); Thorpe, “Antibody Carriers Of Cytotoxic Agents In Cancer Therapy: A Review”, in Monoclonal Antibodies'84: Biological And Clinical Applications, Pinchera et al. (eds.), pp. 475-506 (1985); “Analysis, Results, And Future Prospective Of The Therapeutic Use Of Radiolabeled Antibody In Cancer Therapy”, in Monoclonal Antibodies For Cancer Detection And Therapy, Baldwin et al. (eds.), pp. 303-16 (Academic Press 1985), and Thorpe et al., “The Preparation And Cytotoxic Properties Of Antibody-Toxin Conjugates”, Immunol. Rev., 62:119-58 (1982).

### Bispecific Molecules

In another aspect, the present invention features bispecific molecules comprising an anti-PSMA antibody, or a fragment thereof, of the invention. An antibody of the invention, or antigen-binding portions thereof, can be derivatized or linked to another functional molecule, e.g., another peptide or protein (e.g., another antibody or ligand for a receptor) to generate a bispecific molecule that binds to at least two different binding sites or target molecules. The antibody of the invention may in fact be derivatized or linkd to more than one other functional molecule to generate multispecific molecules that bind to more than two different binding sites and/or target molecules; such multispecific molecules are also intended to be encompassed by the term “bispecific molecule” as used herein. To create a bispecific molecule of the invention, an antibody of the invention can be functionally linked (e.g., by chemical coupling, genetic fusion, noncovalent association or otherwise) to one or more other binding molecules, such as another antibody, antibody fragment, peptide or binding mimetic, such that a bispecific molecule results.

Accordingly, the present invention includes bispecific molecules comprising at least one first binding specificity for PSMA and a second binding specificity for a second target epitope. In a particular embodiment of the invention, the second target epitope is an Fc receptor, e.g., human FcγRI (CD64) or a human Fcα receptor (CD89). Therefore, the invention includes bispecific molecules capable of binding both to FcγR or FcαR expressing effector cells (e.g., monocytes, macrophages or polymorphonuclear cells (PMNs)), and to target cells expressing PSMA. These bispecific molecules target PSMA expressing cells to effector cell and trigger Fc receptor-mediated effector cell activities, such as phagocytosis of an PSMA expressing cells, antibody dependent cell-mediated cytotoxicity (ADCC), cytokine release, or generation of superoxide anion.

In an embodiment of the invention in which the bispecific molecule is multispecific, the molecule can further include a third binding specificity, in addition to an anti-Fc binding specificity and an anti-PSMA binding specificity. In one embodiment, the third binding specificity is an anti-enhancement factor (EF) portion, e.g., a molecule which binds to a surface protein involved in cytotoxic activity and thereby increases the immune response against the target cell. The “anti-enhancement factor portion” can be an antibody, functional antibody fragment or a ligand that binds to a given molecule, e.g., an antigen or a receptor, and thereby results in an enhancement of the effect of the binding determinants for the F_{C} receptor or target cell antigen. The “anti-enhancement factor portion” can bind an F_{C} receptor or a target cell antigen. Alternatively, the anti-enhancement factor portion can bind to an entity that is different from the entity to which the first and second binding specificities bind. For example, the anti-enhancement factor portion can bind a cytotoxic T-cell (e.g. via CD2, CD3, CD8, CD28, CD4, CD40, ICAM-1 or other immune cell that results in an increased immune response against the target cell).

In one embodiment, the bispecific molecules of the invention comprise as a binding specificity at least one antibody, or an antibody fragment thereof, including, e.g., an Fab, Fab′, F(ab′)₂, Fv, or a single chain Fv. The antibody may also be a light chain or heavy chain dimer, or any minimal fragment thereof such as a Fv or a single chain construct as described in Ladner et al. U.S. Pat. No. 4,946,778, the contents of which is expressly incorporated by reference.

In one embodiment, the binding specificity for an Fcγ receptor is provided by a monoclonal antibody, the binding of which is not blocked by human immunoglobulin G (IgG). As used herein, the term “IgG receptor” refers to any of the eight γ-chain genes located on chromosome 1. These genes encode a total of twelve transmembrane or soluble receptor isoforms which are grouped into three Fcγ receptor classes: FcγRI (CD64), FcγRII (CD32), and FcγRIII (CD16). In one preferred embodiment, the Fcγ receptor a human high affinity FcγRI. The human FcγRI is a 72 kDa molecule, which shows high affinity for monomeric IgG (10⁸-10⁹ M⁻¹).

The production and characterization of certain preferred anti-Fcγ monoclonal antibodies are described by Fanger et al. in PCT Publication WO 88/00052 and in U.S. Pat. No. 4,954,617, the teachings of which are fully incorporated by reference herein. These antibodies bind to an epitope of FcγRI, FcγRII or FcγRIII at a site which is distinct from the Fcγ binding site of the receptor and, thus, their binding is not blocked substantially by physiological levels of IgG. Specific anti-FcγRI antibodies useful in this invention are mAb 22, mAb 32, mAb 44, mAb 62 and mAb 197. The hybridoma producing mAb 32 is available from the American Type Culture Collection, ATCC Accession No. HB9469. In other embodiments, the anti-Fcγ receptor antibody is a humanized form of monoclonal antibody 22 (H22). The production and characterization of the H22 antibody is described in Graziano, R. F. et al. (1995) J. Immunol. 155 (10): 4996-5002 and PCT Publication WO 94/10332. The H22 antibody producing cell line was deposited at the American Type Culture Collection under the designation HAO22CL1 and has the accession no. CRL 11177.

In still other preferred embodiments, the binding specificity for an Fc receptor is provided by an antibody that binds to a human IgA receptor, e.g., an Fc-alpha receptor (FcαRI (CD89)), the binding of which is preferably not blocked by human immunoglobulin A (IgA). The term “IgA receptor” is intended to include the gene product of one α-gene (FcαRI) located on chromosome 19. This gene is known to encode several alternatively spliced transmembrane isoforms of 55 to 110 kDa. FcαRI (CD89) is constitutively expressed on monocytes/macrophages, eosinophilic and neutrophilic granulocytes, but not on non-effector cell populations. FcαRI has medium affinity (≈5×10⁷ M⁻¹) for both IgA1 and IgA2, which is increased upon exposure to cytokines such as G-CSF or GM-CSF (Morton, H. C. et al. (1996) Critical Reviews in Immunology 16:423-440). Four FcαRI-specific monoclonal antibodies, identified as A3, A59, A62 and A77, which bind FcαRI outside the IgA ligand binding domain, have been described (Monteiro, R. C. et al. (1992) J. Immunol. 148:1764).

FcαRI and FcγRI are preferred trigger receptors for use in the bispecific molecules of the invention because they are (1) expressed primarily on immune effector cells, e.g., monocytes, PMNs, macrophages and dendritic cells; (2) expressed at high levels (e.g., 5,000-100,000 per cell); (3) mediators of cytotoxic activities (e.g., ADCC, phagocytosis); (4) mediate enhanced antigen presentation of antigens, including self-antigens, targeted to them.

While human monoclonal antibodies are preferred, other antibodies which can be employed in the bispecific molecules of the invention are murine, chimeric and humanized monoclonal antibodies.

The bispecific molecules of the present invention can be prepared by conjugating the constituent binding specificities, e.g., the anti-FcR and anti-PSMA binding specificities, using methods known in the art. For example, each binding specificity of the bispecific molecule can be generated separately and then conjugated to one another. When the binding specificities are proteins or peptides, a variety of coupling or cross-linking agents can be used for covalent conjugation. Examples of cross-linking agents include protein A, carbodiimide, N-succinimidyl-5-acetyl-thioacetate (SATA), 5,5′-dithiobis(2-nitrobenzoic acid) (DTNB), o-phenylenedimaleimide (oPDM), N-succinimidyl-3-(2-pyridyldithio)propionate (SPDP), and sulfosuccinimidyl 4-(N-maleimidomethyl)cyclohaxane-1-carboxylate (sulfo-SMCC) (see e.g., Karpovsky et al. (1984) J. Exp. Med. 160:1686; Liu, M A et al. (1985) Proc. Natl. Acad. Sci. USA 82:8648). Other methods include those described in Paulus (1985) Behring Ins. Mitt. No. 78, 118-132; Brennan et al. (1985) Science 229:81-83), and Glennie et al. (1987) J. Immunol. 139: 2367-2375). Preferred conjugating agents are SATA and sulfo-SMCC, both available from Pierce Chemical Co. (Rockford, Ill.).

When the binding specificities are antibodies, they can be conjugated via sulfhydryl bonding of the C-terminus hinge regions of the two heavy chains. In a particularly preferred embodiment, the hinge region is modified to contain an odd number of sulfhydryl residues, preferably one, prior to conjugation.

Alternatively, both binding specificities can be encoded in the same vector and expressed and assembled in the same host cell. This method is particularly useful where the bispecific molecule is a mAb×mAb, mAb×Fab, Fab×F(ab′)₂ or ligand x Fab fusion protein. A bispecific molecule of the invention can be a single chain molecule comprising one single chain antibody and a binding determinant, or a single chain bispecific molecule comprising two binding determinants. Bispecific molecules may comprise at least two single chain molecules. Methods for preparing bispecific molecules are described for example in U.S. Pat. No. 5,260,203; U.S. Pat. No. 5,455,030; U.S. Pat. No. 4,881,175; U.S. Pat. No. 5,132,405; U.S. Pat. No. 5,091,513; U.S. Pat. No. 5,476,786; U.S. Pat. No. 5,013,653; U.S. Pat. No. 5,258,498; and U.S. Pat. No. 5,482,858.

Binding of the bispecific molecules to their specific targets can be confirmed by, for example, enzyme-linked immunosorbent assay (ELISA), radioimmunoassay (RIA), FACS analysis, bioassay (e.g., growth inhibition), or Western Blot assay. Each of these assays generally detects the presence of protein-antibody complexes of particular interest by employing a labeled reagent (e.g., an antibody) specific for the complex of interest. For example, the FcR-antibody complexes can be detected using e.g., an enzyme-linked antibody or antibody fragment which recognizes and specifically binds to the antibody-FcR complexes. Alternatively, the complexes can be detected using any of a variety of other immunoassays. For example, the antibody can be radioactively labeled and used in a radioimmunoassay (RIA) (see, for example, Weintraub, B., Principles of Radioimmunoassays, Seventh Training Course on Radioligand Assay Techniques, The Endocrine Society, March, 1986, which is incorporated by reference herein). The radioactive isotope can be detected by such means as the use of a γ counter or a scintillation counter or by autoradiography.

### Pharmaceutical Compositions

In another aspect, the present invention provides a composition, e.g. a pharmaceutical composition, containing one or a combination of monoclonal antibodies, or antigen-binding portion(s) thereof, of the present invention, formulated together with a pharmaceutically acceptable carrier. Such compositions may include one or a combination of (e.g., two or more different) antibodies, or immunoconjugates or bispecific molecules of the invention. For example, a pharmaceutical composition of the invention can comprise a combination of antibodies (or immunoconjugates or bispecific) that bind to different epitopes on the target antigen or that have complementary activities.

Pharmaceutical compositions of the invention also can be administered in combination therapy, i.e., combined with other agents. For example, the combination therapy can include an anti-PSMA antibody of the present invention combined with at least one other anti-inflammatory or immunosuppressant agent. Examples of therapeutic agents that can be used in combination therapy are described in greater detail below in the section on uses of the antibodies of the invention.

As used herein, “pharmaceutically acceptable carrier” includes any and all solvents, dispersion media, coatings, antibacterial and antifungal agents, isotonic and absorption delaying agents, and the like that are physiologically compatible. Preferably, the carrier is suitable for intravenous, intramuscular, subcutaneous, parenteral, spinal or epidermal administration (e.g., by injection or infusion). Depending on the route of administration, the active compound, i.e., antibody, immunoconjuage, or bispecific molecule, may be coated in a material to protect the compound from the action of acids and other natural conditions that may inactivate the compound.

The pharmaceutical compounds of the invention may include one or more pharmaceutically acceptable salts. A “pharmaceutically acceptable salt” refers to a salt that retains the desired biological activity of the parent compound and does not impart any undesired toxicological effects (see e.g., Berge, S. M., et al. (1977) J. Pharm. Sci. 66:1-19). Examples of such salts include acid addition salts and base addition salts. Acid addition salts include those derived from nontoxic inorganic acids, such as hydrochloric, nitric, phosphoric, sulfuric, hydrobromic, hydroiodic, phosphorous and the like, as well as from nontoxic organic acids such as aliphatic mono- and dicarboxylic acids, phenyl-substituted alkanoic acids, hydroxy alkanoic acids, aromatic acids, aliphatic and aromatic sulfonic acids and the like. Base addition salts include those derived from alkaline earth metals, such as sodium, potassium, magnesium, calcium and the like, as well as from nontoxic organic amines, such as N,N′-dibenzylethylenediamine, N-methylglucamine, chloroprocaine, choline, diethanolamine, ethylenediamine, procaine and the like.

A pharmaceutical composition of the invention also may include a pharmaceutically acceptable anti-oxidant. Examples of pharmaceutically acceptable antioxidants include: (1) water soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal chelating agents, such as citric acid, ethylenediamine tetraacetic acid (EDTA), sorbitol, tartaric acid, phosphoric acid, and the like.

Examples of suitable aqueous and nonaqueous carriers that may be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents and dispersing agents. Prevention of presence of microorganisms may be ensured both by sterilization procedures, supra, and by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents which delay absorption such as aluminum monostearate and gelatin.

Pharmaceutically acceptable carriers include sterile aqueous solutions or dispersions and sterile powders for the extemporaneous preparation of sterile injectable solutions or dispersion. The use of such media and agents for pharmaceutically active substances is known in the art. Except insofar as any conventional media or agent is incompatible with the active compound, use thereof in the pharmaceutical compositions of the invention is contemplated. Supplementary active compounds can also be incorporated into the compositions.

Therapeutic compositions typically must be sterile and stable under the conditions of manufacture and storage. The composition can be formulated as a solution, microemulsion, liposome, or other ordered structure suitable to high drug concentration. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. In many cases, it will be preferable to include isotonic agents, for example, sugars, polyalcohols such as mannitol, sorbitol, or sodium chloride in the composition. Prolonged absorption of the injectable compositions can be brought about by including in the composition an agent that delays absorption, for example, monostearate salts and gelatin.

Sterile injectable solutions can be prepared by incorporating the active compound in the required amount in an appropriate solvent with one or a combination of ingredients enumerated above, as required, followed by sterilization microfiltration. Generally, dispersions are prepared by incorporating the active compound into a sterile vehicle that contains a basic dispersion medium and the required other ingredients from those enumerated above. In the case of sterile powders for the preparation of sterile injectable solutions, the preferred methods of preparation are vacuum drying and freeze-drying (lyophilization) that yield a powder of the active ingredient plus any additional desired ingredient from a previously sterile-filtered solution thereof.

The amount of active ingredient which can be combined with a carrier material to produce a single dosage form will vary depending upon the subject being treated, and the particular mode of administration. The amount of active ingredient which can be combined with a carrier material to produce a single dosage form will generally be that amount of the composition which produces a therapeutic effect. Generally, out of one hundred percent, this amount will range from about 0.01 percent to about ninety-nine percent of active ingredient, preferably from about 0.1 percent to about 70 percent, most preferably from about 1 percent to about 30 percent of active ingredient in combination with a pharmaceutically acceptable carrier.

Dosage regimens are adjusted to provide the optimum desired response (e.g., a therapeutic response). For example, a single bolus may be administered, several divided doses may be administered over time or the dose may be proportionally reduced or increased as indicated by the exigencies of the therapeutic situation. It is especially advantageous to formulate parenteral compositions in dosage unit form for ease of administration and uniformity of dosage. Dosage unit form as used herein refers to physically discrete units suited as unitary dosages for the subjects to be treated; each unit contains a predetermined quantity of active compound calculated to produce the desired therapeutic effect in association with the required pharmaceutical carrier. The specification for the dosage unit forms of the invention are dictated by and directly dependent on (a) the unique characteristics of the active compound and the particular therapeutic effect to be achieved, and (b) the limitations inherent in the art of compounding such an active compound for the treatment of sensitivity in individuals.

For administration of the antibody, the dosage ranges from about 0.0001 to 100 mg/kg, and more usually 0.01 to 5 mg/kg, of the host body weight. For example dosages can be 0.3 mg/kg body weight, 1 mg/kg body weight, 3 mg/kg body weight, 5 mg/kg body weight or 10 mg/kg body weight or within the range of 1-10 mg/kg. An exemplary treatment regime entails administration once per week, once every two weeks, once every three weeks, once every four weeks, once a month, once every 3 months or once every three to 6 months. Preferred dosage regimens for an anti-PSMA antibody of the invention include 1 mg/kg body weight or 3 mg/kg body weight via intravenous administration, with the antibody being given using one of the following dosing schedules: (i) every four weeks for six dosages, then every three months; (ii) every three weeks; (iii) 3 mg/kg body weight once followed by 1 mg/kg body weight every three weeks.

In some methods, two or more monoclonal antibodies with different binding specificities are administered simultaneously, in which case the dosage of each antibody administered falls within the ranges indicated. Antibody is usually administered on multiple occasions. Intervals between single dosages can be, for example, weekly, monthly, every three monthly or yearly. Intervals can also be irregular as indicated by measuring blood levels of antibody to the target antigen in the patient. In some methods, dosage is adjusted to achieve a plasma antibody concentration of about 1-1000 μg/ml and in some methods about 25-300 μg/ml.

Alternatively, antibody can be administered as a sustained release formulation, in which case less frequent administration is required. Dosage and frequency vary depending on the half-life of the antibody in the patient. In general, human antibodies show the longest half life, followed by humanized antibodies, chimeric antibodies, and nonhuman antibodies. The dosage and frequency of administration can vary depending on whether the treatment is prophylactic or therapeutic. In prophylactic applications, a relatively low dosage is administered at relatively infrequent intervals over a long period of time. Some patients continue to receive treatment for the rest of their lives. In therapeutic applications, a relatively high dosage at relatively short intervals is sometimes required until progression of the disease is reduced or terminated, and preferably until the patient shows partial or complete amelioration of symptoms of disease. Thereafter, the patient can be administered a prophylactic regime.

Actual dosage levels of the active ingredients in the pharmaceutical compositions of the present invention may be varied so as to obtain an amount of the active ingredient which is effective to achieve the desired therapeutic response for a particular patient, composition, and mode of administration, without being toxic to the patient. The selected dosage level will depend upon a variety of pharmacokinetic factors including the activity of the particular compositions of the present invention employed, or the ester, salt or amide thereof, the route of administration, the time of administration, the rate of excretion of the particular compound being employed, the duration of the treatment, other drugs, compounds and/or materials used in combination with the particular compositions employed, the age, sex, weight, condition, general health and prior medical history of the patient being treated, and like factors well known in the medical arts.

A “therapeutically effective dosage” of an anti-PSMA antibody of the invention preferably results in a decrease in severity of disease symptoms, an increase in frequency and duration of disease symptom-free periods, or a prevention of impairment or disability due to the disease affliction. For example, for the treatment of PSMA+tumors, a “therapeutically effective dosage” preferably inhibits cell growth or tumor growth by at least about 20%, more preferably by at least about 40%, even more preferably by at least about 60%, and still more preferably by at least about 80% relative to untreated subjects. The ability of a compound to inhibit tumor growth can be evaluated in an animal model system predictive of efficacy in human tumors. Alternatively, this property of a composition can be evaluated by examining the ability of the compound to inhibit, such inhibition in vitro by assays known to the skilled practitioner. A therapeutically effective amount of a therapeutic compound can decrease tumor size, or otherwise ameliorate symptoms in a subject. One of ordinary skill in the art would be able to determine such amounts based on such factors as the subject's size, the severity of the subject's symptoms, and the particular composition or route of administration selected.

A composition of the present invention can be administered via one or more routes of administration using one or more of a variety of methods known in the art. As will be appreciated by the skilled artisan, the route and/or mode of administration will vary depending upon the desired results. Preferred routes of administration for antibodies of the invention include intravenous, intramuscular, intradermal, intraperitoneal, subcutaneous, spinal or other parenteral routes of administration, for example by injection or infusion. The phrase “parenteral administration” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion.

Alternatively, an antibody of the invention can be administered via a non-parenteral route, such as a topical, epidermal or mucosal route of administration, for example, intranasally, orally, vaginally, rectally, sublingually or topically.

The active compounds can be prepared with carriers that will protect the compound against rapid release, such as a controlled release formulation, including implants, transdermal patches, and microencapsulated delivery systems. Biodegradable, biocompatible polymers can be used, such as ethylene vinyl acetate, polyanhydrides, polyglycolic acid, collagen, polyorthoesters, and polylactic acid. Many methods for the preparation of such formulations are patented or generally known to those skilled in the art. See, e.g., Sustained and Controlled Release Drug Delivery Systems, J. R. Robinson, ed., Marcel Dekker, Inc., New York, 1978.

Therapeutic compositions can be administered with medical devices known in the art. For example, in a preferred embodiment, a therapeutic composition of the invention can be administered with a needleless hypodermic injection device, such as the devices disclosed in U.S. Pat. Nos. 5,399,163; 5,383,851; 5,312,335; 5,064,413; 4,941,880; 4,790,824; or 4,596,556. Examples of well-known implants and modules useful in the present invention include: U.S. Pat. No. 4,487,603, which discloses an implantable micro-infusion pump for dispensing medication at a controlled rate; U.S. Pat. No. 4,486,194, which discloses a therapeutic device for administering medicants through the skin; U.S. Pat. No. 4,447,233, which discloses a medication infusion pump for delivering medication at a precise infusion rate; U.S. Pat. No. 4,447,224, which discloses a variable flow implantable infusion apparatus for continuous drug delivery; U.S. Pat. No. 4,439,196, which discloses an osmotic drug delivery system having multi-chamber compartments; and U.S. Pat. No. 4,475,196, which discloses an osmotic drug delivery system. These patents are incorporated herein by reference. Many other such implants, delivery systems, and modules are known to those skilled in the art.

In certain embodiments, the human monoclonal antibodies of the invention can be formulated to ensure proper distribution in vivo. For example, the blood-brain barrier (BBB) excludes many highly hydrophilic compounds. To ensure that the therapeutic compounds of the invention cross the BBB (if desired), they can be formulated, for example, in liposomes. For methods of manufacturing liposomes, see, e.g., U.S. Pat. Nos. 4,522,811; 5,374,548; and 5,399,331. The liposomes may comprise one or more moieties which are selectively transported into specific cells or organs, thus enhance targeted drug delivery (see, e.g., V. V. Ranade (1989) J. Clin. Pharmacol. 29:685). Exemplary targeting moieties include folate or biotin (see, e.g., U.S. Pat. No. 5,416,016 to Low et al.); mannosides (Umezawa et al., (1988) Biochem. Biophys. Res. Commun. 153:1038); antibodies (P. G. Bloeman et al. (1995) FEBS Lett. 357:140; M. Owais et al. (1995) Antimicrob. Agents Chemother. 39:180); surfactant protein A receptor (Briscoe et al. (1995) Am. J. Physiol. 1233:134); p 120 (Schreier et al. (1994) J. Biol. Chem. 269:9090); see also K. Keinanen; M. L. Laukkanen (1994) FEBS Lett. 346:123; J. J. Killion; I. J. Fidler (1994) Immunomethods 4:273.

### Uses and Methods of the Invention

The antibodies, antibody compositions and methods of the present invention have numerous in vitro and in vivo diagnostic and therapeutic utilities involving the diagnosis and treatment of disorders involving expression of PSMA. For example, these molecules can be administered to cells in culture, e.g. in vitro or ex vivo, or to human subjects, e.g., in vivo, to treat, prevent and to diagnose a variety of disorders. As used herein, the term “subject” is intended to include human and non-human animals. Non-human animals includes all vertebrates, e.g., mammals and non-mammals, such as non-human primates, sheep, dogs, cats, cows, horses, pigs, chickens, avians, amphibians, and reptiles. Preferred subjects include human patients having disorders associated with PSMA expression. When antibodies to PSMA are administered together with another agent, the two can be administered in either order or simultaneously.

Suitable routes of administering the antibody compositions (e.g., antibody or immunoconjugate) of the invention in vivo and in vitro are well known in the art and can be selected by those of ordinary skill. For example, the antibody compositions can be administered by injection (e.g., intravenous or subcutaneous). Suitable dosages of the molecules used will depend on the age and weight of the subject and the concentration and/or formulation of the antibody composition.

In one embodiment, the antibodies of the invention can be initially tested for binding activity associated with therapeutic or diagnostic use in vitro. For example, compositions of the invention can be tested using ELISA and flow cytometric assays. Moreover, the activity of these molecules in triggering at least one effector-mediated effector cell activity, including inhibiting the growth of and/or killing of cells expressing PSMA can be assayed. Protocols for assaying for effector cell-mediated ADCC are described in the Examples below.

A. Detection Methods

In one embodiment, the antibodies of the invention can be used to detect levels of PSMA, or levels of cells which contain PSMA on their membrane surface, which levels can then be linked to certain disease symptoms.

In a particular embodiment, the invention provides methods for detecting the presence of PSMA in a sample, or measuring the amount of PSMA on the surface of cells, comprising contacting the sample, and a control sample, with an antibody, or an antigen binding portion thereof, which specifically binds to PSMA, under conditions that allow for formation of a complex between the antibody or portion thereof and PSMA. The formation of a complex is then detected, wherein a difference complex formation between the sample compared to the control sample is indicative the presence of PSMA in the sample. For example, standard detection methods, well-known in the art, such as ELISA and flow cytometic assays, can be performed using the compositions of the invention.

Accordingly, in one aspect, the invention further provides methods for detecting the presence of PSMA (e.g., human PSMA) in a sample, or measuring the amount of PSMA, comprising contacting the sample, and a control sample, with an antibody of the invention, or an antigen binding portion thereof, which specifically binds to PSMA, under conditions that allow for formation of a complex between the antibody or portion thereof and PSMA. The formation of a complex is then detected, wherein a difference in complex formation between the sample compared to the control sample is indicative of the presence of PSMA in the sample.

The compositions of the invention can also be used to target cells expressing PSMA, for example for labeling such cells. For such use, the binding agent can be linked to a molecule that can be detected. Thus, the invention provides methods for localizing ex vivo or in vitro cells expressing PSMA. The detectable label can be, e.g., a radioisotope, a fluorescent compound, an enzyme, or an enzyme co-factor.

B. Inhibition of Growth of PSMA+ Cells

The antibodies can be used to inhibit growth of cells expressing PSMA which, in turn, can be linked to the prevention or amelioration of certain disease symptoms associated with PSMA expression. Differences in PSMA expression during a disease state as compared to a non-disease state can be determined by contacting a test sample from a subject suffering from the disease and a control sample with the anti-PSMA antibody under conditions that allow for the formation of a complex between the antibody and PSMA. Any complexes formed between the antibody and PSMA are detected and compared in the sample and the control.

For example, the antibodies can be used to elicit in vivo or in vitro one or more of the following biological activities: to inhibit the growth of and/or kill a cell expressing PSMA; to mediate phagocytosis or ADCC of a cell expressing PSMA in the presence of human effector cells; to inhibit shedding of soluble PSMA. As discussed herein, the antibodies of the invention exhibit enhanced ADCC activity as compared to the fucosylated form of the antibody.

Accordingly, in another aspect, the invention provides a method of inhibiting growth of PSMA⁺ cells comprising contacting said cells with an anti-PSMA antibody under conditions sufficient to induce antibody-dependent cellular cytoxicity (ADCC) of said cells. The cells can be, for example, tumor cells. In a preferred embodiment, the anti-PSMA antibody is a human antibody.

In one embodiment, the antibodies, or binding portions thereof, of the present invention can be used to modulate PSMA levels on target cells, such as by capping and eliminating receptors on the cell surface. Mixtures of anti-Fc receptor antibodies can also be used for this purpose.

Target-specific effector cells, e.g., effector cells linked to compositions of the invention can also be used as therapeutic agents. Effector cells for targeting can be human leukocytes such as macrophages, neutrophils or monocytes. Other cells include eosinophils, natural killer cells and other IgG- or IgA-receptor bearing cells. If desired, effector cells can be obtained from the subject to be treated. The target-specific effector cells, can be administered as a suspension of cells in a physiologically acceptable solution. The number of cells administered can be in the order of 108-109 but will vary depending on the therapeutic purpose. In general, the amount will be sufficient to obtain localization at the target cell, e.g., a tumor cell expressing PSMA, and to effect cell killing by, e.g., phagocytosis. Routes of administration can also vary.

Therapy with target-specific effector cells can be performed in conjunction with other techniques for removal of targeted cells. For example, anti-tumor therapy using the compositions of the invention and/or effector cells armed with these compositions can be used in conjunction with chemotherapy. Additionally, combination immunotherapy may be used to direct two distinct cytotoxic effector populations toward tumor cell rejection.

C. Use of Immunoconjugates and Combination Therapy

In one embodiment, immunoconjugates of the invention can be used to target compounds (e.g., therapeutic agents, labels, cytotoxins, radiotoxins immunosuppressants, etc.) to cells which have PSMA cell surface molecules by linking such compounds to the antibody. Thus, the invention also provides methods for localizing ex vivo or in vitro cells expressing PSMA (e.g., with a detectable label, such as a radioisotope, a fluorescent compound, an enzyme, or an enzyme co-factor). Alternatively, the immunoconjugates can be used to kill cells which have PSMA cell surface receptors by targeting cytotoxins or radiotoxins to PSMA, such as to PSMA-expressing tumor cells to thereby eliminate the tumor cell.

In other embodiments, the subject can be additionally treated with an agent that modulates, e.g., enhances or inhibits, the expression or activity of Fcγ or Fcγ receptors by, for example, treating the subject with a cytokine. Preferred cytokines for administration during treatment include of granulocyte colony-stimulating factor (G-CSF), granulocyte-macrophage colony-stimulating factor (GM-CSF), interferon-γ (IFN-γ), and tumor necrosis factor (TNF).

In another embodiment, patients treated with antibody compositions of the invention can be additionally administered prior to, simultaneously with, or following administration of an antibody of the invention) with another therapeutic agent, such as a cytotoxic or radiotoxic agent, which enhances or augments the therapeutic effect of the human antibodies. The antibody can be linked to the agent (as an immunocomplex) or can be administered separate from the agent. In the latter case (separate administration), the antibody can be administered before, after or concurrently with the agent or can be co-administered with other known therapies, e.g., an anti-cancer therapy, e.g., radiation. Such therapeutic agents include, among others, anti-neoplastic agents such as doxorubicin (adriamycin), cisplatin bleomycin sulfate, carmustine, chlorambucil, and cyclophosphamide hydroxyurea which, by themselves, are only effective at levels which are toxic or subtoxic to a patient. Cisplatin is intravenously administered as a 100 mg/ml dose once every four weeks and adriamycin is intravenously administered as a 60-75 mg/ml dose once every 21 days. Co-administration of the anti-PSMA antibodies, or antigen binding fragments thereof, of the present invention with chemotherapeutic agents provides two anti-cancer agents which operate via different mechanisms which yield a cytotoxic effect to human tumor cells. Such co-administration can solve problems due to development of resistance to drugs or a change in the antigenicity of the tumor cells which would render them unreactive with the antibody.

D. Treatment of Cancer

PSMA has been shown to be expressed on tumor cells, such as prostate carcinoma tumor cells, and also has been shown to be expressed on vascular endothelial cells proximate to cancerous cells, such as urothelial cancerous cells, colon cancerous cells, rectal cancerous cells, lung cancerous cells, breast cancerous cells and metastatic adenocarcinoma cancerous cells of the liver (see U.S. Pat. No. 6,136,311). Accordingly, the antibodies of the invention can be used to treat cancer by inhibiting growth of PSMA-expressing tumor cells or by inhibiting growth of vascular endothelial cells proximate to tumor cells. Thus, in another embodiment, the present invention provides a method of inhibiting growth of a tumor in a subject, wherein cells of the tumor or vascular endothelial cells proximate to the tumor are PSMA⁺, in which an anti-PSMA antibody of the invention is administered to the subject such that growth of the tumor is inhibited. For human subjects, the antibody preferably is a humanized or human antibody. In a preferred embodiment, the tumor cells are prostate tumor cells. In other embodiments, the tumor cells are from cancers such as colon, renal, rectal, urothelial, breast, bladder, liver, pancreas or melanoma.

The treatment methods of the invention involve administering to a subject an antibody composition of the present invention in an amount effective to treat or prevent the disorder. The antibody composition can be administered alone or another therapeutic agent, such as a cytotoxic or a radiotoxic agent (conjugated to or administered with the antibody), which acts in conjunction with or synergistically with the antibody composition to treat or prevent the disease associated with PSMA expression.

The treatment methods of the invention also involve administering to a subject an anti-PSMA antibody in combination with another agent, such as an anti-tumor agent, which acts in conjunction with or synergistically with the antibody composition to treat or prevent the disease associated with PSMA expression. As shown herein, use of the anti-PSMA antibody 7F12 in combination with Taxotere® (Docetaxel) resulted in inhibition of tumor growth in an animal model and cured the animals from tumor related cachexia (see Example 8). Without intending to be limited by mechanism, it is believed that the use of an anti-tumor agent causes damage in the tumor mass, thus, allowing improved penetration of antibody, effector cells or an alternative effector component, leading to more effective cytotoxicity. In one embodiment, use of an anti-tumor agent causes damage in the tumor mass, thus, allowing effector cells to access the tumor, leading to a more effective antibody dependent cell-mediated cytotoxicity (ADCC) of the tumor cells. In another embodiment, use of an anti-tumor agent, e.g., a microtubule inhibitor, causes tumor cells to be intrinsically more susceptible to anti-PSMA mediated killing. In one embodiment of the invention, the synergistic or additive effect obtained by the administration of an anti-PSMA antibody and an anti-tumor agent is independent of, i.e., not caused by, the reversal of the apical polarity of the PSMA antigen to the basolateral plasma membrane.

As used herein, the term “anti-PSMA antibody” includes any antibody that specifically binds to the human prostate specific membrane antigen. Examples of such antibodies include the antibodies described herein, the antibodies described in U.S. patent application Ser. No. 10/059,989 and PCT Publication No. WO 03064606A3, or the antibodies described in U.S. Provisional Application No. 60/660,431. The entire contents of each of the foregoing applications are incorporated herein by reference.

As used herein, the term “anti-tumor agent” includes any agent that may be used to destroy or assist in destroying (e.g., partially or totally) a tumor. In one embodiment, the anti-tumor agent is capable of causing damage in the tumor mass, thereby allowing effector cells to access the tumor more readily, leading to a more effective antibody dependent cell-mediated cytotoxicity (ADCC) of the tumor cells. The term “anti-tumor agent” includes chemotherapeutic agents, angiogenesis inhibitors, microtubule blocking agents, immunomodulatory agents, DNA intercalators/cross linkers, DNA synthesis inhibitors, DNA-RNA transcription regulators, enzyme inhibitors, and gene regulators.

The term “chemotherapeutic agent” includes any agent that may be used to treat cancer, e.g., prostate cancer. Chemotherapeutic agents include alkylating agents, antimetabolites, plant alkaloids, antitumor antibiotics and steroid hormones. Specific examples of chemotherapeutic agents include, but are not limited to, all-trans retinoic acid, Aminoglutethimide, Azacitidine, Azathioprine, Bleomycin (Blenoxane), Busulfan (Myeleran), Carboplatin, Carboplatinum (Paraplatin), Carmustine (BCNU), Capecitabine, CCNU (Lomustine), Chlorambucil (Leulceran), 2-chlorodeoxyadenosine (2-CDA; Cladribine, Leustatin), Cis-platinum (Platinol), Cisplatin (cis-DDP), Cisplatin bleomycin sulfate, Chlorambucil, Cyclophosphamide (Cytoxanl CTX), Cyclophosphamide hydroxyurea, Cytarabine (Ara-C; cytosine arabinoside), Daunorubicin (Cerubidine), Dacarbazine (DTIC; dimethyltriazenoimidazolecarboxamide), Dactinomycin (actinomycin D), Daunorubicin (daunomycin; rubidomycin), Diethylstilbestrol, Docetaxel (Taxotere), Doxifluridine, Doxorubicin (Adriamycin), Epirubicin, Ethinyl estradoil, Etopaside (VP-16, VePesid), Fluorouracil (5-Fu; Floxuridine, fluorodeoxyuridine; FUdR), Fludarabine (Fludara), Flutamide, Fluoxymesterone, Gemcitabine (Gemzar), Herceptin (Trastuzumab; anti-HER 2 monoclonal antibody), Hydroxyurea (Hydrea), Hydroxyprogesterone caproate, Idarubicin, Ifosfamide (Ifex), Interferon alpha, Irinotecan (CPT-11), L-Asparaginase, Leuprolide, Mechlorethamine, Medroxyprogesterone acetate, Megestrol acetate, Melphelan (Alkeran), Mercaptopurine (6-mercaptopurine; 6-MP), Methotrexate (MTX; amethopterin), Mitomycin (mitomycin C), Mitotane (o,p′-DDD), Mitoxantrone (Novantrone), Oxaliplatin, Paclitaxel (Taxol), Pemetrexed, Pentostatin (2-deoxycoformycin), Plicamycin (mithramycin), Prednisone, Procarbazine (Matulane; N-methylhydrazine, MIH), Rituxin (Rituximap), Semustine (Methyl-CCNU), Streptozocin, Taxol, Tamoxifen, Teniposide, Tertiposide, Testosterone propionate, Thioguanine (6-thioguanine; TG), Thiotepa, Tomudex (Raltitrexed), Topotecan (Hycamtin; (S)-10-[(dimethylamino) methyl]-4-ethyl-4,9-dihydroxy-1H-pyrano[3′, 4′), Treosulfan (Ovastat), Valrubicin, Vinblastine (VLB; Velban), Vincristine (Oncovin), Vindesine, and Vinorelbine (Navelbine).

The term “microtubule blocking agent” includes any agent that is capable of disrupting the normal organization and dynamics of microtubules. Examples of microtubule blocking agents include, but are not limited to, taxanes (Taxol® (Paclitaxel), Taxotere® (docetaxel)), vinca alkaloids (vinblastine, vincristine (Oncovin), Vindesine (Eldisine, Fildesin), Vinorelbine (Navelbine)), 2-methoxyestradiol (2ME2), estramustine, epothilones, Colchicine, Dolastatin 15, Nocodazole, Podophyllotoxin, Rhizoxin.

The terms “angiogenesis inhibitor” and “anti-angiogenic agent” are used interchangeably herein and include any agent that is capable of preventing or inhibiting the formation of blood vessels. Specific examples of angiogenesis inhibitors include, but are not limited to, Angiostatin K1-3, Arresten, aaAT, Canstatin, DL-α-Difluoromethyl-ornithine, Endostatin, Fumagillin, Genistein, Minocycline, Staurosporine, (±)-Thalidomide, and Tumstatin.

The term “immunomodulating agent” includes any agent that modulates, e.g., stimulates, an immune response. Examples of immunomodulating agents include antibodies, such as anti-PD1 antibodies and anti-CTLA-4 antibodies, alone or in combination, described, inter-alia, in U.S. Provisional Application No. 60/679,466, filed May 9, 2005; in PCT Publication WO 01/14424; in U.S. Provisional Application 60/738,434, filed Nov. 21, 2005; and in U.S. Provisional application Ser. No. ______, filed Dec. 8, 2005 (Attorney Docket No. MEDX-0124US2 or 04280/1203401-US1), the entire contents of each of which are expressly incorporated herein by reference.

Additional immunomodulating agents that may be used in the methods of the present invention include antibodies that block a costimulatory signal, (e.g., CD28 or ICOS), antibodies that activate an inhibitory signal via CTLA4, and/or antibodies against other immune cell markers (e.g., CD40, CD40 ligand, or cytokines), fusion proteins (e.g., CTLA4-Fc, PD-1-Fc), and immunosuppressive drugs, (e.g., rapamycin, cyclosporine A or FK506). Other examples of immunomodulating agents include phosphorothiolate oligodeoxyribonucleotide (1018 ISS), GVAX (a GM-CSF gene vaccine), interleukins (e.g., interleukin-1, -2, -3, -4, -5, -6, -7 (CYT 99 07), -8, -9, -10, -11, -13, -14, -15, -16, -17, -18, -19, -20, -21, -22, -23, -24, -25, -26, -27, -28, -29 and -30), e.g., recombinant interleukin antibodies, e.g. IL-2, e.g., Aldesleulin, e.g., recombinant interleukins (e.g., recombinant interleukin-21 (rIL-21)), e.g., IL-11, e.g., oprelvekin, e.g., interleukin receptor antagonists, e.g., IL-1 receptor antagonist, e.g., anakinra, glucocerebrosidase, e.g., Imiglucerase, macrophage activating factor, macrophage peptide, B cell factor, and T cell factor.

Examples of DNA intercalators/cross linkers include, but are not limited to, Bleomycin, Carboplatin, Carmustine, Chlorambucil, Cyclophosphamide, cis-Diammineplatinum (II) dichloride (Cisplatin), Melphalan, Mitoxantrone, and Oxaliplatin.

Examples of DNA synthesis inhibitors include, but are not limited to, (±)-Amethopterin (Methotrexate), 3-Amino-1,2,4-benzotriazine 1,4-dioxide, Aminopterin, Cytosine β-D-arabinofuranoside, 5-Fluoro-5′-deoxyuridine, 5-Fluorouracil, Ganciclovir, Hydroxyurea, and Mitomycin C.

Examples of DNA-RNA transcription regulators include, but are not limited to, Actinomycin D, Daunorubicin, Doxorubicin, Homoharringtonine, and Idarubicin.

Examples of enzyme inhibitors include, but are not limited to, S(+)-Camptothecin, Curcumin, (−)-Deguelin, 5,6-Dichlorobenz-imidazole 1-β-D-ribofuranoside, Etoposide, Formestane, Fostriecin, Hispidin, 2-Imino-1-imidazoli-dineacetic acid (Cyclocreatine), Mevinolin, Trichostatin A, Tyrphostin AG 34, and Tyrphostin AG 879.

Examples of gene regulators include, but are not limited to, 5-Aza-2′-deoxycytidine, 5-Azacytidine, Cholecalciferol (Vitamin D3), 4-Hydroxytamoxifen, Melatonin, Mifepristone, Raloxifene, all trans-Retinal (Vitamin A aldehyde), Retinoic acid, all trans (Vitamin A acid), 9-cis-Retinoic Acid, 13-cis-Retinoic acid, Retinol (Vitamin A), Tamoxifen, and Troglitazone.

The methods of the invention also involve administering to a subject an immunoconjugate (comprising an anti-PSMA antibody, or antigen-binding portion thereof, linked to a therapeutic agent, such as a cytotoxin or a radioactive isotope) in combination with an anti-tumor agent, which acts in conjunction with or synergistically with the antibody composition to treat or prevent the disease associated with PSMA expression.

The methods of the invention further involve administering to a subject a bispecific molecule (comprising an anti-PSMA antibody, or antigen-binding portion thereof, linked to a second functional moiety having a different binding specificity than the antibody) in combination with an anti-tumor agent, which acts in conjunction with or synergistically with the antibody composition to treat or prevent the disease associated with PSMA expression. The anti-tumor agent may be administered in any therapeutically effective dosage known in the art or as described herein (see pharmaceutical compositions).

The anti-PSMA antibody may be administered in combination with a single anti-tumor agent. The anti-PSMA antibody may also be administered in combination with two or more anti-tumor agents.

The anti-PSMA antibody and the anti-tumor agent may be administered simultaneously. For example, the anti-PSMA antibody and the anti-tumor agent may be administered together in a single pharmacutical formulation. In another embodiment, the anti-PSMA antibody and the anti-tumor agent may be administered at the same time as two or more separate pharmaceutical formulations.

The anti-PSMA antibody and the anti-tumor agent may also be administered at different times. For example, the anti-tumor agent(s) may be administered prior to administration of the anti-PSMA antibody (e.g., about 1, 2, 3, 4, 5, 6, 7, 8, 9, 10, 15, 20, 24 or 48 hours prior to administration of the anti-PSMA antibody). Alternatively, the anti-PSMA antibody may be administered prior to administration of the anti-tumor agent(s) (e.g., about 1, 2, 3, 4, 5, 6, 7, 8, 9, 10, 15, 20, 24 or 48 hours prior to administration of the anti-tumor agent(s)). In one embodiment, the anti-PSMA antibody may be administered in combination with an anti-tumor agent according to the dosing schedule delineated herein in Table 1.

### Kits

Also within the scope of the invention are kits comprising an antibody of the invention and instructions for use. The kit can further contain one or more additional reagents, such as an immunostimulatory reagent, a cytotoxic agent or a radiotoxic agent, or one or more additional antibodies of the invention (e.g., an antibody having a complementary activity which binds to an epitope in the PSMA antigen distinct from the first antibody). Kits typically include a label indicating the intended use of the contents of the kit. The term label includes any writing, or recorded material supplied on or with the kit, or which otherwise accompanies the kit.

The present invention is further illustrated by the following examples which should not be construed as further limiting. The contents of all figures and all references, patents and published patent applications cited throughout this application are expressly incorporated herein by reference.

### Example 1

### Generation of Human Monoclonal Antibodies Against PSMA

### Antigen

Immunization protocols utilized as antigen cells of the PSMA expressing prostate cancer cell line LNCaP (ATCC CRL-1740).

### Transgenic HuMab Mice

Fully human monoclonal antibodies to PSMA were prepared using the HCol2 strain of HuMab transgenic mice, which expresses human antibody genes. In this mouse strains, the endogenous mouse kappa light chain gene has been homozygously disrupted as described in Chen et al. (1993) EMBO J. 12:811-820 and the endogenous mouse heavy chain gene has been homozygously disrupted as described in Example 1 of PCT Publication WO 01/09187. Furthermore, this mouse strain carries a human kappa light chain transgene, KCo5, as described in Fishwild et al. (1996) Nature Biotechnology 14:845-851, and a human heavy chain transgene, HCo12, as described in Example 2 of PCT Publication WO 01/09187.

### HuMab Immunizations:

To generate fully human monoclonal antibodies to PSMA, HuMab mice were immunized with LNCaP cells expressing PSMA as antigen. General immunization schemes for HuMab mice are described in Lonberg, N. et al (1994) Nature 368(6474): 856-859; Fishwild, D. et al. (1996) Nature Biotechnology 14: 845-851 and PCT Publication WO 98/24884. The mice were 6-16 weeks of age upon the first infusion of antigen. 5-10×10⁶ cells were used to immunize the HuMab mice intraperitonealy (IP), subcutaneously (Sc) or via footpad injection.

Transgenic mice were immunized twice with antigen in complete Freund's adjuvant or Ribi adjuvant IP, followed by 3-21 days IP (up to a total of 11 immunizations) with the antigen in incomplete Freund's or Ribi adjuvant. The immune response was monitored by retroorbital bleeds. The plasma was screened by ELISA (as described below), and mice with sufficient titers of anti-PSMA human immunoglobulin were used for fusions. Mice were boosted intravenously with antigen 3 days before sacrifice and removal of the spleen. Typically, 10-35 fusions for each antigen were performed. Several dozen mice were immunized for each antigen.

### Selection of HuMab Mice Producing Anti-PSMA Antibodies:

To select HuMab mice producing antibodies that bound PSMA, sera from immunized mice were screened for binding to the PSMA expressing prostate cancer cell line LNCaP, but not to a negative prostate cancer cell line by flow cytometry. Briefly, the binding of anti-PSMA antibodies was assessed by incubating LNCaP cells with the anti-PSMA antibody at a concentration of 20 μg/ml. The cells were washed and binding was detected with a FITC-labeled anti-human IgG Ab. Flow cytometric analyses were performed using a FACScan flow cytometry (Becton Dickinson, San Jose, Calif.). Antibodies that bound to the PSMA expressing LNCaP cells but not the non-PSMA expressing prostate cancer cells were further tested for binding to PSMA by ELISA, as described by Fishwild, D. et al. (1996). Briefly, microtiter plates were coated with purified PSMA at 1-2 μg/ml in PBS, 100 μl/wells incubated 4° C. overnight then blocked with 200 μl/well of 5% fetal bovine serum in PBS/Tween (0.05%). Dilutions of sera from PSMA-immunized mice were added to each well and incubated for 1-2 hours at ambient temperature. The plates were washed with PBS/Tween and then incubated with a goat-anti-human IgG polyclonal antibody conjugated with horseradish peroxidase (HRP) for 1 hour at room temperature. After washing, the plates were developed with ABTS substrate (Sigma, A-1888, 0.22 mg/ml) and analyzed by spectrophotometer at OD 415-495. Mice that developed the highest titers of anti-PSMA antibodies were used for fusions. Fusions were performed as described below and hybridoma supernatants were tested for anti-PSMA activity by ELISA.

### Generation of Hybridomas Producing Human Monoclonal Antibodies to PSMA:

The mouse splenocytes, isolated from the HuMab mice, were fused with PEG to a mouse myeloma cell line based upon standard protocols. The resulting hybridomas were then screened for the production of antigen-specific antibodies. Single cell suspensions of splenocytes from immunized mice were fused to one-fourth the number of SP2/0 nonsecreting mouse myeloma cells (ATCC, CRL 1581) with 50% PEG (Sigma). Cells were plated at approximately 1×10⁵/well in flat bottom microtiter plate, followed by about two week incubation in selective medium containing 10% fetal bovine serum, 10% P388D1 (ATCC, CRL TIB-63) conditioned medium, 3-5% origen (IGEN) in DMEM (Mediatech, CRL 10013, with high glucose, L-glutamine and sodium pyruvate) plus 5 mM HEPES, 0.055 mM 2-mercaptoethanol, 50 mg/ml gentamycin and 1×HAT (Sigma, CRL P-7185). After 1-2 weeks, cells were cultured in medium in which the HAT was replaced with HT. Individual wells were then screened by ELISA (described above) for human anti-PSMA monoclonal IgG antibodies. Once extensive hybridoma growth occurred, medium was monitored usually after 10-14 days. The antibody-secreting hybridomas were replated, screened again and, if still positive for human IgG, anti-PSMA monoclonal antibodies were subcloned at least twice by limiting dilution. The stable subclones were then cultured in vitro to generate small amounts of antibody in tissue culture medium for further characterization.

Hybridoma clones 1C3, 2A10, 2F5, 2C6, were selected for further analysis.

### Example 2

### Structural Characterization of Human Monoclonal Antibodies 1C3, 2A10, 2F5, and 2C6

The cDNA sequences encoding the heavy and light chain variable regions of the 1C3, 2A10, 2F5, and 2C6 monoclonal antibodies were obtained from the 1C3, 2A10, 2F5, and 2C6 hybridomas, respectively, using standard PCR techniques and were sequenced using standard DNA sequencing techniques.

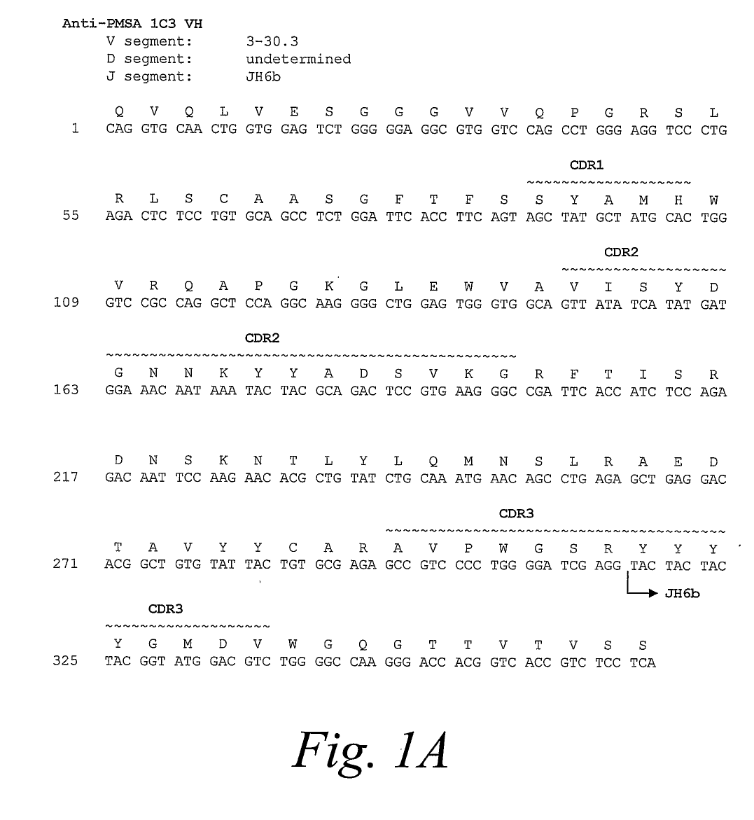

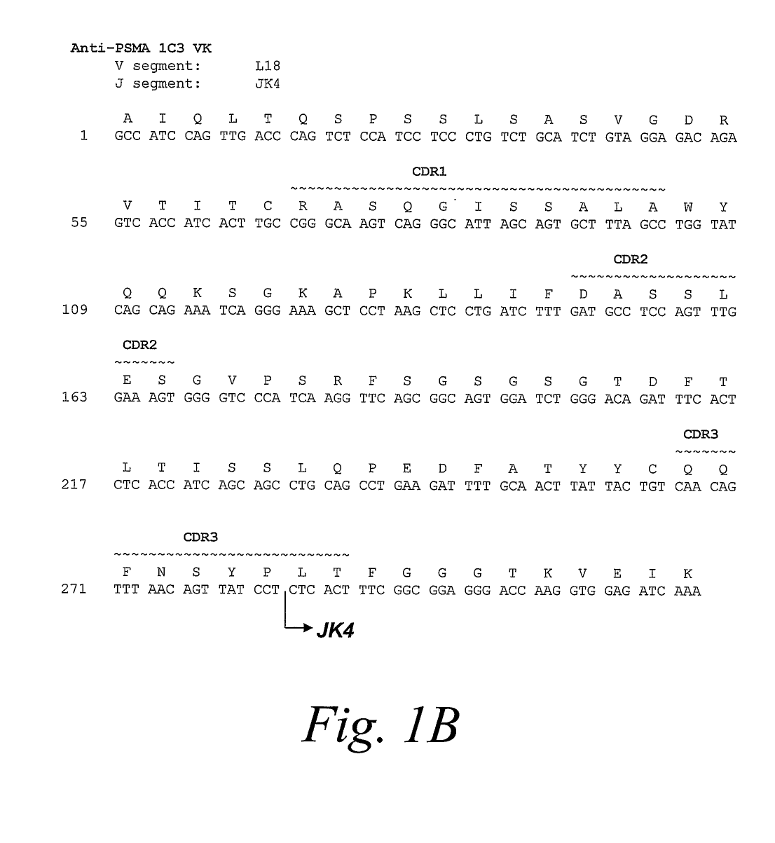

The nucleotide and amino acid sequences of the heavy chain variable region of 1C3 are shown in FIG. 1A and in SEQ ID NO: 33 and 1, respectively.

The nucleotide and amino acid sequences of the light chain variable region of 1C3 are shown in FIG. 1B and in SEQ ID NO: 37 and 5, respectively.

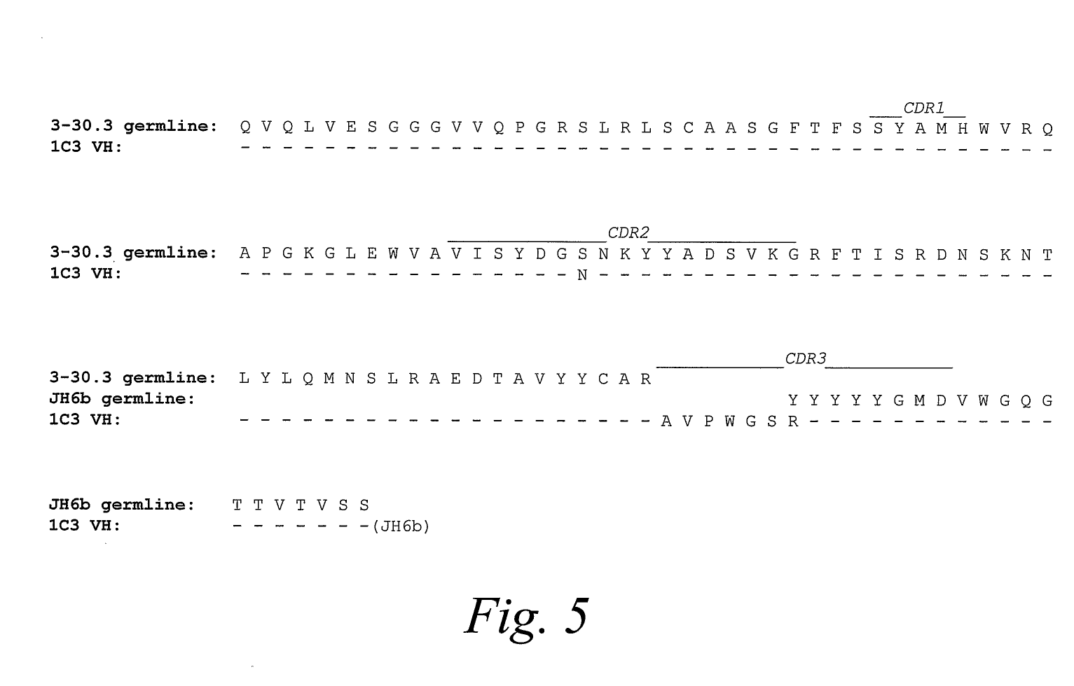

Comparison of the 1C3 heavy chain immunoglobulin sequence to the known human germline immunoglobulin heavy chain sequences demonstrated that the 1C3 heavy chain utilizes a VH segment from human germline VH 3-30.3, an undetermined D segment, and a JH segment from human germline JH 6b. The alignment of the 1C3 VH sequence to the germline VH 3-30.3 sequence is shown in FIG. 5. Further analysis of the 1C3 VH sequence using the Kabat system of CDR region determination led to the delineation of the heavy chain CDR1, CDR2 and CD3 regions as shown in FIGS. 1A and 5, and in SEQ ID NOs: 9, 13 and 17, respectively.

Comparison of the 1C3 light chain immunoglobulin sequence to the known human germline immunoglobulin light chain sequences demonstrated that the 1C3 light chain utilizes a VL segment from human germline VK L18 and a JK segment from human germline JK 4. The alignment of the 1C3 VL sequence to the germline VK L18 sequence is shown in FIG. 7. Further analysis of the 1C3 VL sequence using the Kabat system of CDR region determination led to the delineation of the light chain CDR1, CDR2 and CD3 regions as shown in FIGS. 1B and 7, and in SEQ ID NOs:21, 25 and 29, respectively.

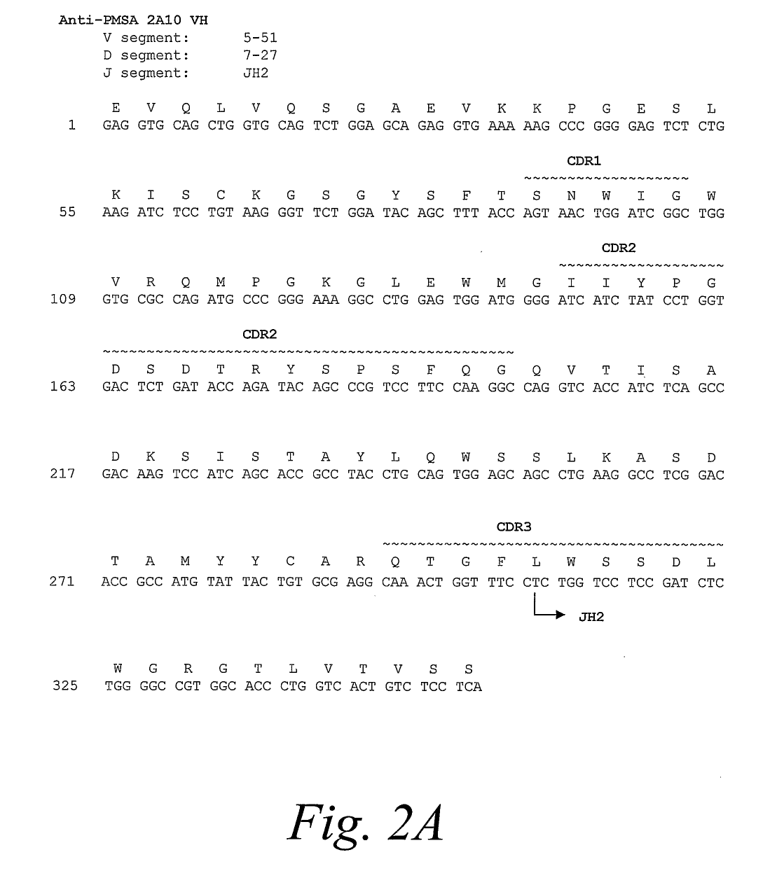

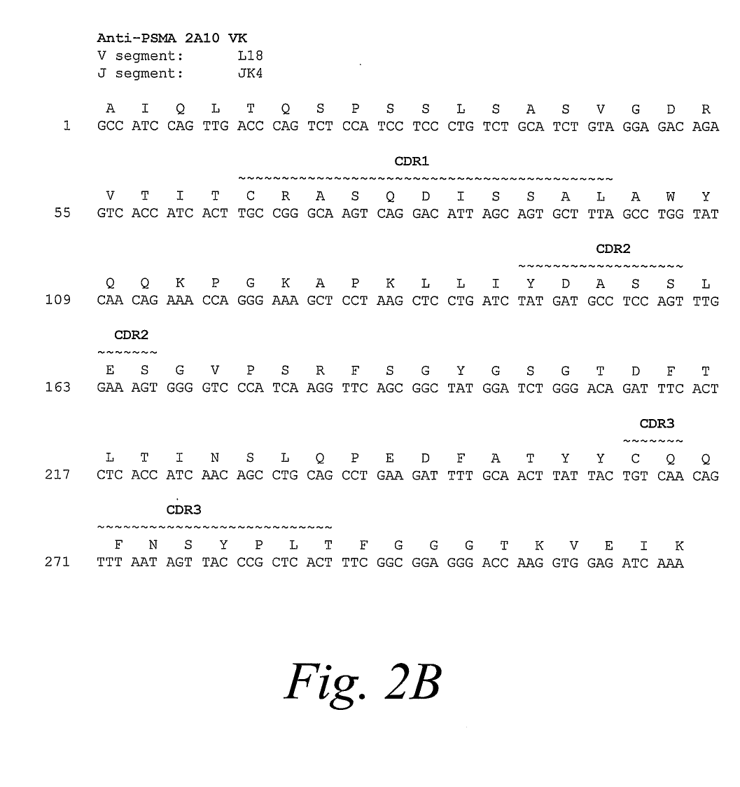

The nucleotide and amino acid sequences of the heavy chain variable region of 2A10 are shown in FIG. 2A and in SEQ ID NO: 34 and 2, respectively.

The nucleotide and amino acid sequences of the light chain variable region of 2A10 are shown in FIG. 2B and in SEQ ID NO: 38 and 6, respectively.

Comparison of the 2A10 heavy chain immunoglobulin sequence to the known human germline immunoglobulin heavy chain sequences demonstrated that the 2A10 heavy chain utilizes a VH segment from human germline VH 5-51, a D segment from human germline 7-27, and a JH segment from human germline JH 2. The alignment of the 2A10 VH sequence to the germline VH 5-51 sequence is shown in FIG. 6. Further analysis of the 2A10 VH sequence using the Kabat system of CDR region determination led to the delineation of the heavy chain CDR1, CDR2 and CD3 regions as shown in FIGS. 2A and 6, and in SEQ ID NOs: 10, 14 and 18, respectively.

Comparison of the 2A10 light chain immunoglobulin sequence to the known human germline immunoglobulin light chain sequences demonstrated that the 2A10 light chain utilizes a VL segment from human germline VK L18 and a JK segment from human germline JK 4. The alignment of the 2A10 VL sequence to the germline VK L18 sequence is shown in FIG. 7. Further analysis of the 2A10 VL sequence using the Kabat system of CDR region determination led to the delineation of the light chain CDR1, CDR2 and CD3 regions as shown in FIGS. 2B and 7, and in SEQ ID NOs: 22, 26 and 30, respectively.

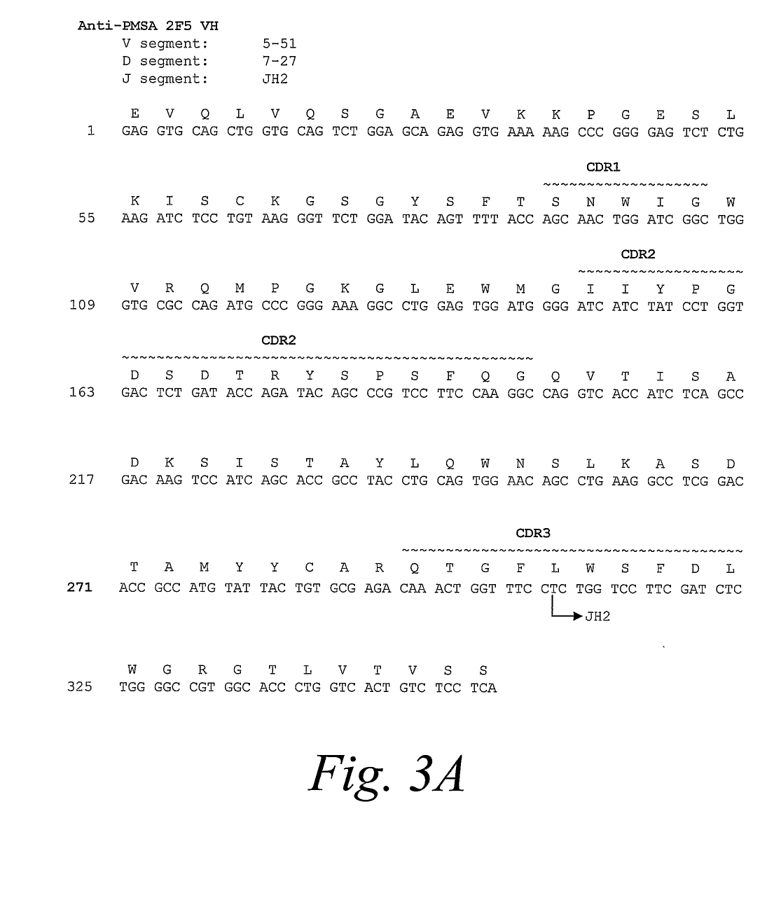

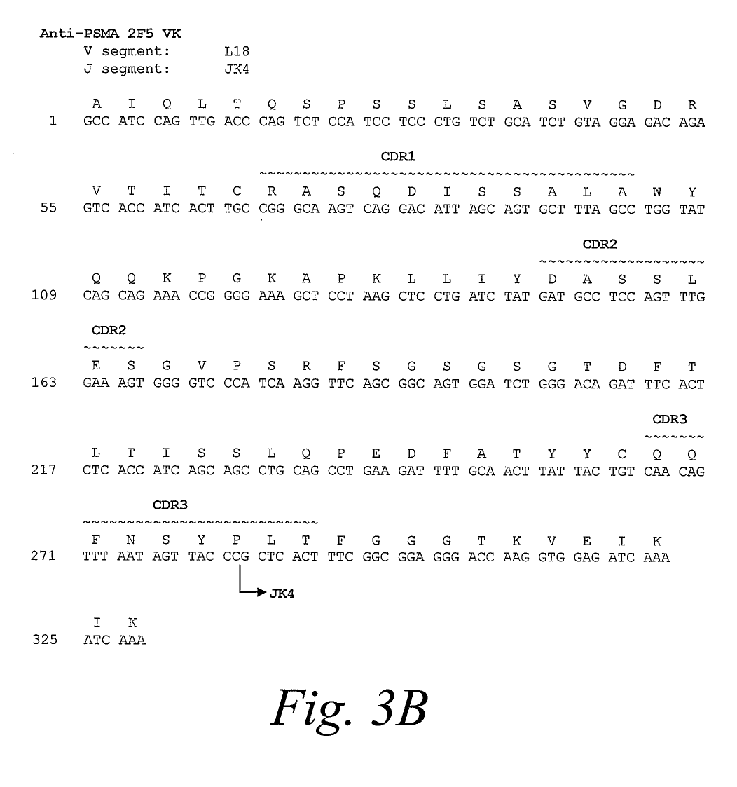

The nucleotide and amino acid sequences of the heavy chain variable region of 2F5 are shown in FIG. 3A and in SEQ ID NO: 35 and 3, respectively.

The nucleotide and amino acid sequences of the light chain variable region of 2F5 are shown in FIG. 3B and in SEQ ID NO: 39 and 7, respectively.

Comparison of the 2F5 heavy chain immunoglobulin sequence to the known human germline immunoglobulin heavy chain sequences demonstrated that the 2F5 heavy chain utilizes a VH segment from human germline VH 5-51, a D segment from human germline 7-27, and a JH segment from human germline JH 2. The alignment of the 2F5 VH sequence to the germline VH 5-51 sequence is shown in FIG. 6. Further analysis of the 2F5 V_{H} sequence using the Kabat system of CDR region determination led to the delineation of the heavy chain CDR1, CDR2 and CD3 regions as shown in FIGS. 3A and 6, and in SEQ ID NOs: 11, 15 and 19, respectively.

Comparison of the 2F5 light chain immunoglobulin sequence to the known human germline immunoglobulin light chain sequences demonstrated that the 2F5 light chain utilizes a VL segment from human germline VK L18 and a JK segment from human germline JK 4. The alignment of the 2F5 VL sequence to the germline VK L18 sequence is shown in FIG. 7. Further analysis of the 2F5 VL sequence using the Kabat system of CDR region determination led to the delineation of the light chain CDR1, CDR2 and CD3 regions as shown in FIGS. 3B and 7, and in SEQ ID NOs: 23, 27 and 31, respectively.

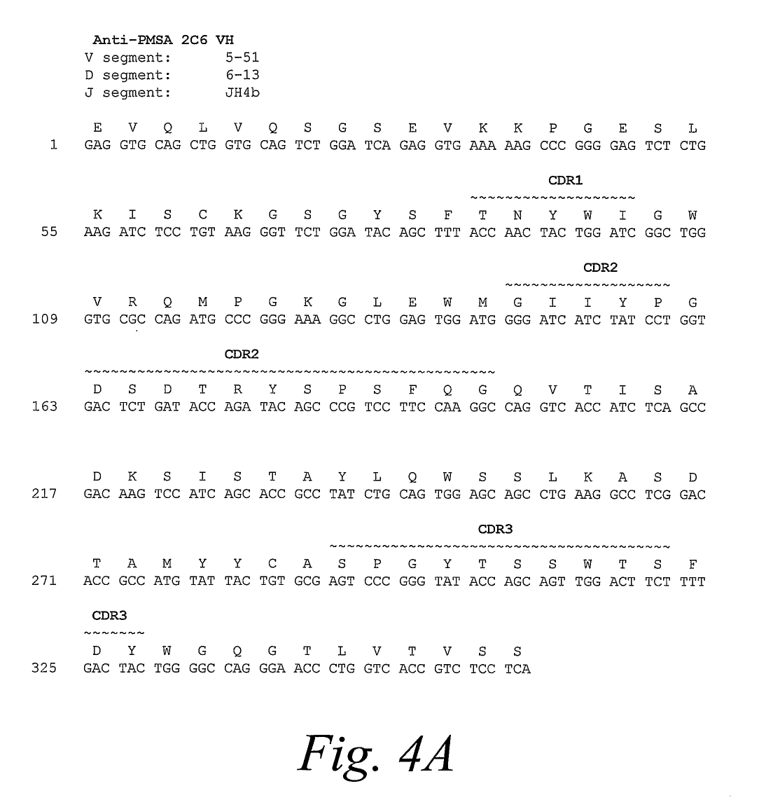

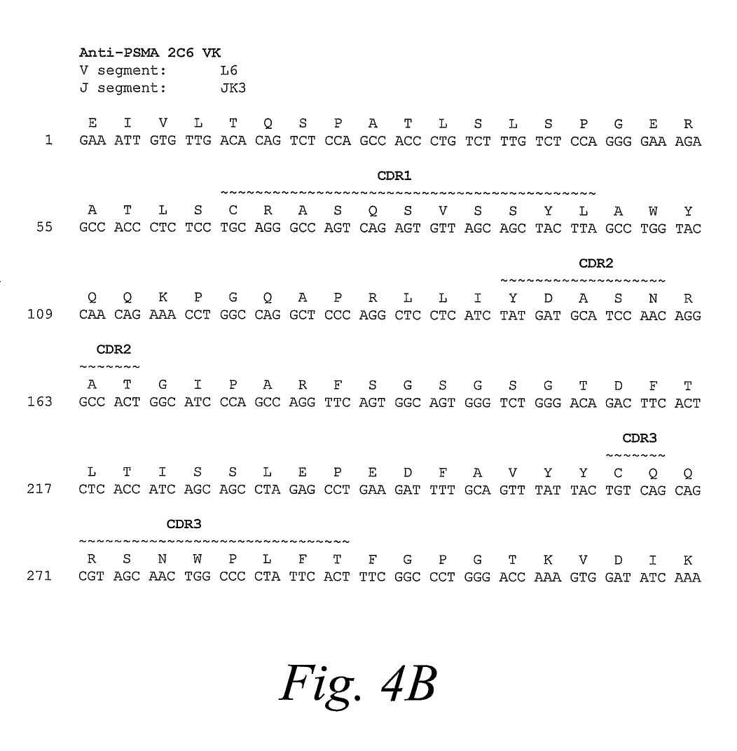

The nucleotide and amino acid sequences of the heavy chain variable region of 2C6 are shown in FIG. 4A and in SEQ ID NO: 36 and 4, respectively.

The nucleotide and amino acid sequences of the light chain variable region of 2C6 are shown in FIG. 4B and in SEQ ID NO: 40 and 8, respectively.

Comparison of the 2C6 heavy chain immunoglobulin sequence to the known human germline immunoglobulin heavy chain sequences demonstrated that the 2C6 heavy chain utilizes a VH segment from human germline VH 5-51, a D segment from human germline 6-13, and a JH segment from human germline JH 4b. The alignment of the 2C6 VH sequence to the germline VH 5-51 sequence is shown in FIG. 6. Further analysis of the 2C6 VH sequence using the Kabat system of CDR region determination led to the delineation of the heavy chain CDR1, CDR2 and CD3 regions as shown in FIGS. 4A and 6, and in SEQ ID NOs: 12, 16 and 20, respectively.

Comparison of the 2C6 light chain immunoglobulin sequence to the known human germline immunoglobulin light chain sequences demonstrated that the 2C6 light chain utilizes a VL segment from human gerniline VK L6 and a JK segment from human germline JK 3. The alignment of the 2C6 VL sequence to the germline VK L6 sequence is shown in FIG. 8. Further analysis of the 2C6 VL sequence using the Kabat system of CDR region determination led to the delineation of the light chain CDR1, CDR2 and CD3 regions as shown in FIGS. 4B and 8, and in SEQ ID NOs: 24, 28 and 32, respectively.

### Example 3

### Characterization of Binding Specificity of Anti-PSMA Human Monoclonal Antibodies

In this example, binding specificity was examined by flow cytometry on a prostate cancer cell line expressing PSMA and by ELISA using purified PSMA.

### Binding Specificity by Flow Cytometry

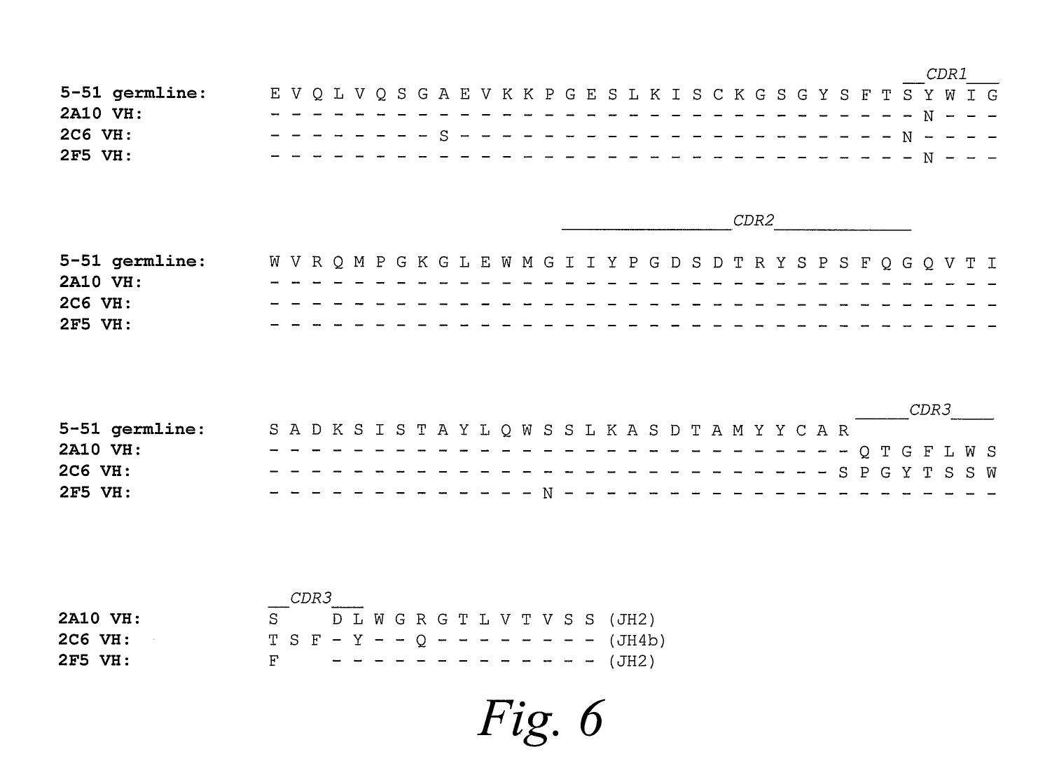

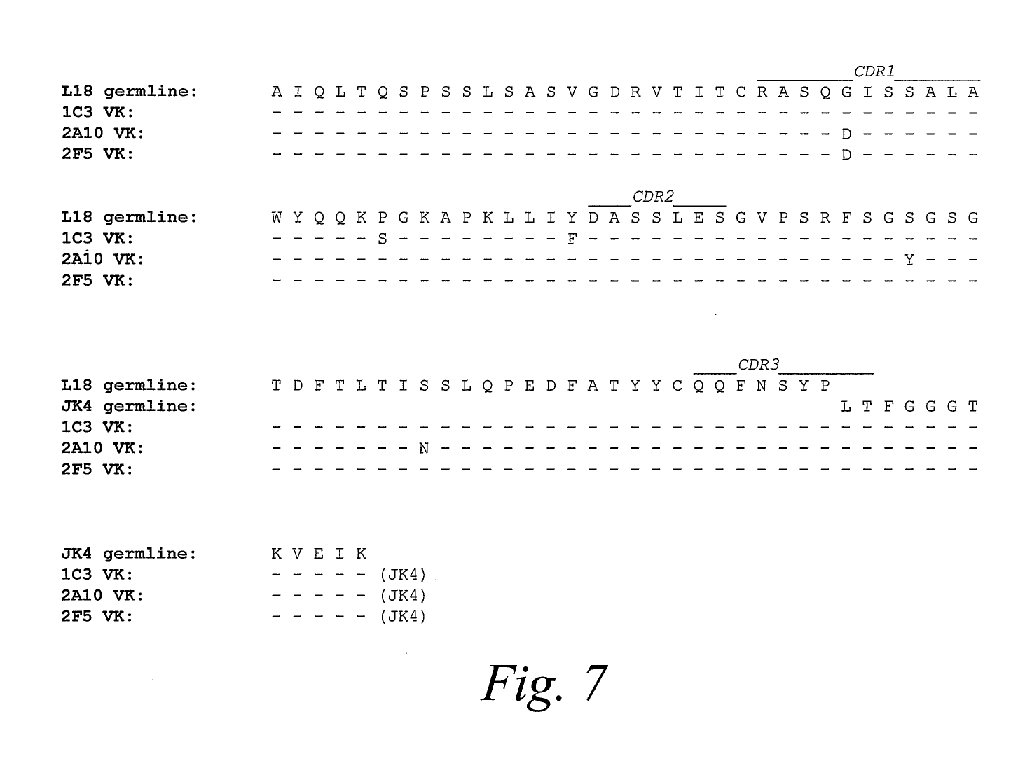

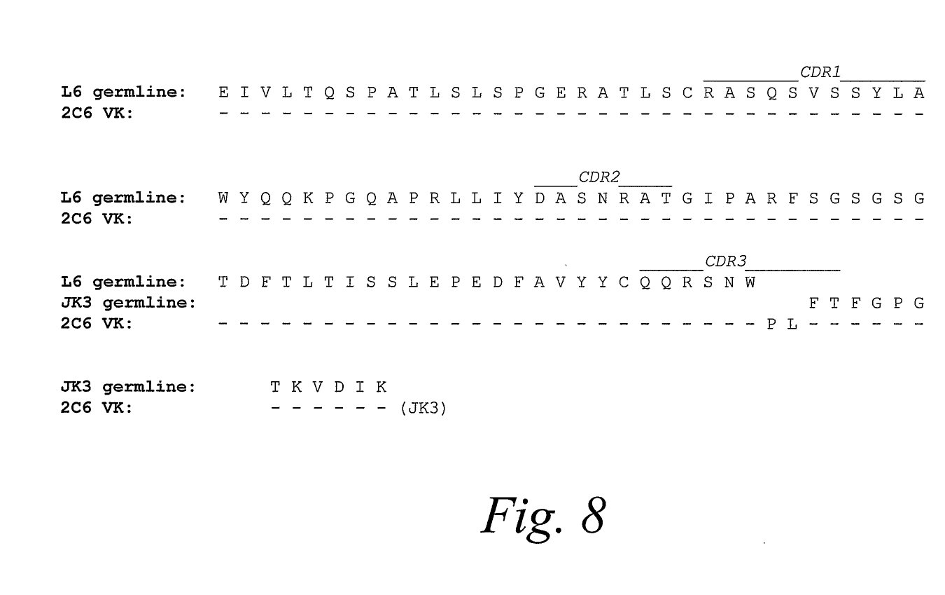

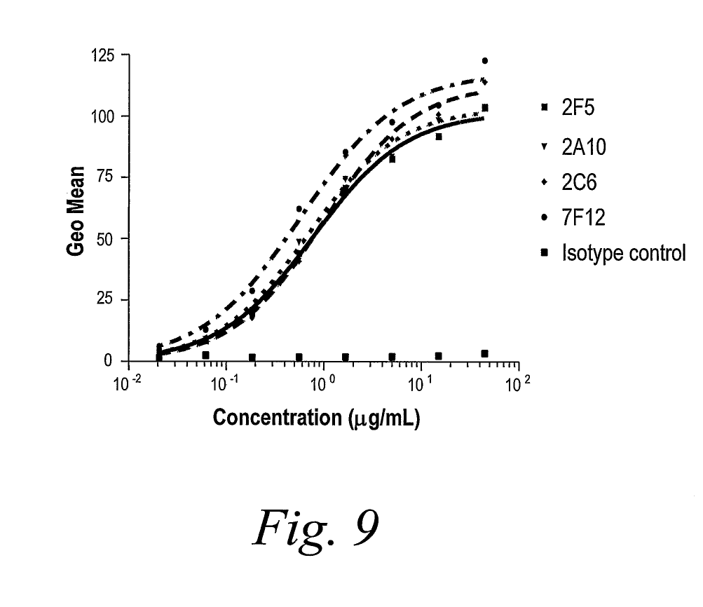

The human PSMA-expressing prostate cancer cell line LNCaP was used to determine the specificity of anti-PSMA human monoclonal antibodies by flow cytometry. Binding of the 2F5, 2A10, and 2C6 anti-PSMA human monoclonal antibodies was assessed by incubating the LNCaP cells with the anti-PSMA human monoclonal antibodies at different concentrations. The cells were washed and binding was detected with a FITC-labeled anti-human IgG Ab. Flow cytometric analyses were performed using a FACScan flow cytometry (Becton Dickinson, San Jose, Calif.). The human anti-PSMA monoclonal antibody 7F12 (as described in PCT Publication WO 03/064606) was used as a positive control and a non-PSMA specific isotype control antibody was used as a negative control. The results are depicted in FIG. 9. The anti-PSMA human monoclonal antibodies 2F5, 2A10, and 2C6 bound specifically to the PSMA-expressing LNCaP cells.

### Binding Specificity by ELISA

A comparison of anti-PSMA antibodies on binding to immunopurified PSMA was performed by standard ELISA to examine the specificity of binding for PSMA.

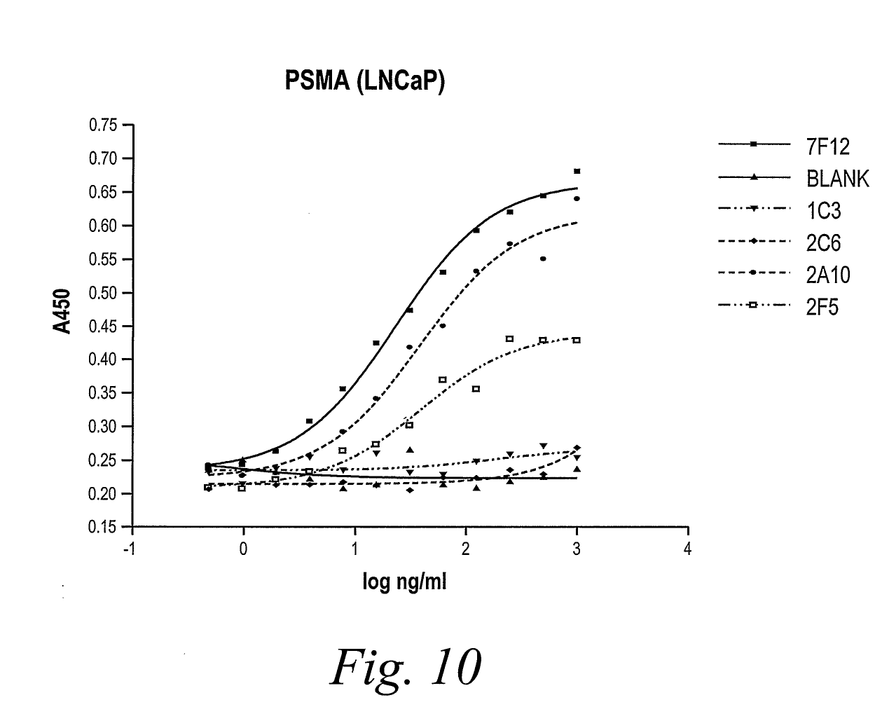

PSMA was immunopurified from LNCaP cells and tested for binding against the anti-PSMA human monoclonal antibodies 1C3, 2A10, 2F5, and 2C6. Standard ELISA procedures were performed. The anti-PSMA human monoclonal antibodies were added at a concentration of 5 μg/ml and titrated down at 1:2 serial dilutions. Goat-anti-human IgG (kappa chain-specific) polyclonal antibody conjugated with horseradish peroxidase (HRP) was used as secondary antibody. The human anti-PSMA monoclonal antibody 7F12 was used as a positive control and a blank was used as a negative control. The results are shown in FIG. 10. The anti-PSMA human monoclonal antibodies 2A10 and 2F5 bound with high specificity to PSMA. The anti-PSMA human monoclonal antibodies 1C3 and 2C6 exhibited detectable but only low level binding to PSMA in an ELISA assay, suggesting that these antibodies bind to a hindered epitope in an ELISA assay.

### Example 4

### Scatchard Analysis of Binding Affinity of Anti-PSMA Monoclonal Antibodies

The binding affinity of the 2A10 antibody for the PSMA-expressing prostate cancer LNCaP cell line was tested using a Scatchard analysis.

LNCaP cells were obtained from ATCC(CRL-1740) and grown in RPMI media containing 10% fetal bovine serum (FBS). The cells were trypsinized and washed once in Tris based binding buffer (24 mM Tris pH 7.2, 137 mM NaCl, 2.7 mM KCl, 2mM Glucose, 1 mM CaCl₂, 1 mM MgCl₂, 0.1% BSA) and the cells were adjusted to 2×10⁶ cells/ml in binding buffer. Millipore plates (MAFB NOB) were coated with 1% nonfat dry milk in water and stored a 4° C. overnight. The plates were washed three times with 0.2 ml of binding buffer. Fifty microliters of buffer alone was added to the maximum binding wells (total binding). Twenty-five microliters of buffer alone was added to the control wells (non-specific binding). Varying concentration of ¹²⁵I-anti-PSMA antibody was added to all wells in a volume of 25 μl . Varying concentrations of unlabeled antibody at 100 fold excess was added in a volume of 25 μl to control wells and 25 μl of LNCaP cells (2×10⁶ cells/ml) in binding buffer were added to all wells. The plates were incubated for 2 hours at 200 RPM on a shaker at 4° C. At the completion of the incubation the Millipore plates were washed three times with 0.2 ml of cold wash buffer (24 mM Tris pH 7.2, 500 mM NaCl, 2.7 mM KCl, 2mM Glucose, 1 mM CaCl₂, 1 mM MgCl₂, 0.1% BSA). The filters were removed and counted in a gamma counter. Evaluation of equilibrium binding was performed using single site binding parameters with the Prism software (San Diego, Calif.).

Using the above scatchard binding assay, the K_{D} of the 2A10 antibody for LNCaP cells was approximately 0.8 nM.

### Example 5

### Epitope Competition Binding Assay

The anti-PSMA monoclonal antibody 2A10 was compared to a known anti-PSMA antibody, 7F12 (described in PCT Publication WO 03/064606) to examine whether the two antibodies bound to the same epitope region using a competition assay.

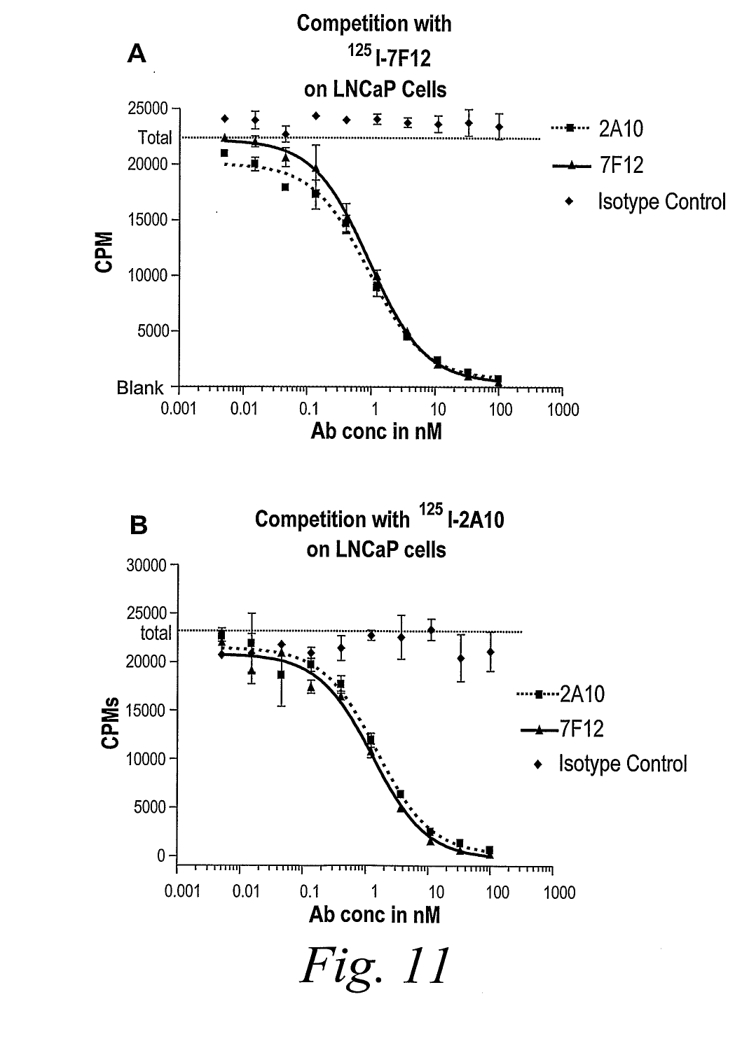

LNCaP cells were obtained from ATCC(CRL-1740) and grown in RPMI media containing 10% fetal bovine serum (FBS). The cells were trypsinized and washed once in Tris based binding buffer (24 mM Tris pH 7.2, 137 mM NaCl, 2.7 mM KCl, 2 mM Glucose, 1 mM CaCl₂, IMM MgCl₂, 0.1% BSA) and the cells were adjusted to 2×10⁶ cells/ml in binding buffer. Millipore plates (MAFB NOB) were coated with 1% nonfat dry milk in water and stored a 4° C. overnight. The plates were washed three times with 0.2 ml of binding buffer. Twenty-five microliters of buffer alone was added to the wells (non-specific binding). A fixed concentration of ¹²⁵I-anti-PSMA antibody was added to all wells in a volume of 25 μl. Varying concentrations of unlabeled antibody was added to wells in a volume of 25 μl and 25 μl of LNCaP cells (2×10⁶ cells/ml) in binding buffer were added to all wells. The plates were incubated for 2 hours at 200 RPM on a shaker at 4° C. At the completion of the incubation the Millipore plates were washed three times with 0.2 ml of cold wash buffer (24 mM Tris pH 7.2, 500mM NaCl, 2 mM KCl, 2 mM Glucose, 1 mM CaCl₂, 1 mM MgCl₂, 0.1% BSA). The filters were removed and counted in a gamma counter. Evaluation of equilibrium binding was performed using single site binding parameters with the Prism software (San Diego, Calif.). An isotype control antibody was used as a negative control. The results of competition binding against ¹²⁵I-2A10 are shown in FIG. 11A and competition binding against ¹²⁵I-7F12 are shown in FIG. 11B. The results show that addition of unlabeled 7F12 antibody inhibits the binding of labeled 2A10 to LNCaP cells and the addition of unlabeled 2A10 antibody inhibits the binding of labeled 7F12 to LNCaP cells. This demonstrates that the 2A10 and 7F12 anti-PSMA antibodies bind to the same or a very similar epitope on PSMA.

### Example 6

### Internalization of anti-PSMA Monoclonal Antibody

Anti-PSMA HuMAbs were tested for the ability to internalize into PSMA-expressing prostate cancer cells using a Hum-Zap internalization assay. Hum-Zap tests for internalization of a primary human antibody through binding of a secondary antibody with affinity for human IgG conjugated to the toxin saporin.

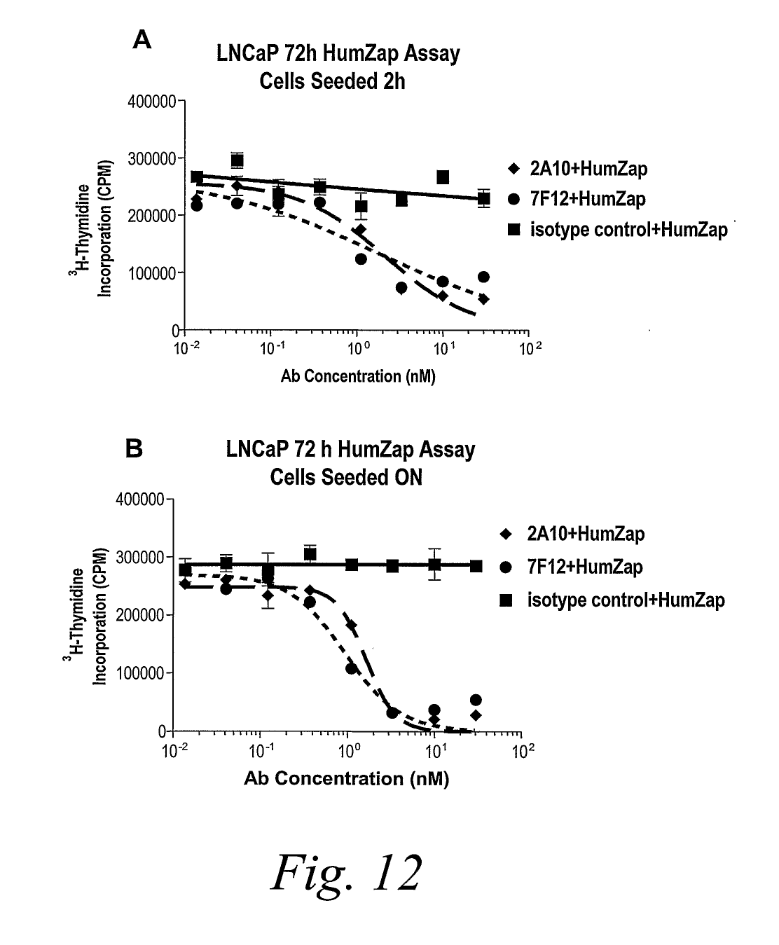

The PSMA-expressing prostate cancer cell line LNCaP was seeded at 2.5×10⁴ cells/well in 100 μl wells either overnight or the following day for a two hour period. Either the anti-PSMA antibody 2A10 or 7F12 were added to the wells at a starting concentration of 30 nM and titrated down at 1:3 serial dilutions. An isotype control antibody that is non-specific for PSMA was used as a negative control. The Hum-Zap (Advanced Targeting Systems, IT-22-25) was added at a concentration of 11 nM and plates were allowed to incubate for 48 hours. The plates were then pulsed with 1.0 μCi of ³H-thymidine for 24 hours, harvested and read in a Top Count Scintillation Counter (Packard Instruments). The results are shown in FIG. 12. The anti-PSMA antibody 2A10 showed an antibody concentration dependent decrease in ³H-thymidine incorporation in PSMA-expressing LNCap prostate cancer cells. This data demonstrates that the anti-PSMA antibody 2A10 internalizes into a prostate cancer cell line.

### Example 7

### Thermostability of Anti-PSMA Monoclonal Antibodies by Differential Scanning Calorimetry

The thermal stability of the anti-PSMA monoclonal antibody 2A10 was compared to the 7F12 antibody using calorimetric analysis of the melting temperature of the antibody.

Calorimetric measurements of melting Temperatures™ were carried out on a VP-Capillary DSC differential scanning microcalorimeter platform that is combined with an autosampler (MicroCal LLC, Northampton, Mass., USA). Sample cell volume is 0.144 mL. Denaturation data on the glycosylated and deglycosylated forms of the antibodies was obtained by heating the samples, at a concentration of 2.3 μM, from 30 to 95° C. at a rate of 1° C./min. The protein samples were present in phosphate-buffered saline (PBS) at pH 7.4. The same buffer was used in the reference cell to obtain the molar heat capacity by comparison. The observed thermograms were baseline corrected and normalized data analyzed based on a 2-step model, using the software Origin v7.0. The clone 2A10 has higher thermostability) with a Tₘ of 71.43° C., compared to 63.05° C. obtained for the clone 7F12.

### Example 8

### Treatment of in Vivo Tumor Xenografts with a Combination of Taxotere® and the 7F12 Antibody

The anti-tumor efficacy of the anti-PSMA 7F12 antibody in combination with Taxotere® (docetaxel) was tested on LNCaP human prostate carcinoma xenografts grown in male CB17.SCID mice.

LNCaP prostate cancer cells expressing high levels of PSMA were obtained from ATCC (Cat# CRL-1740) and expanded in vitro following ATCC instruction. 8 week-old male CB17.SCID mice from Taconic were implanted subcutaneously in the right flank with 2.5×10⁶ LNCaP cells in 0.2 ml of PBS/Matrigel (1:1) per mouse. Mice were weighed and measured for tumor volume using an electronic caliper twice weekly starting three weeks post implantation. Tumor volumes were calculated as height×width×length. Mice with vascularized tumors (determined by the appearance of the tumors) of 180 mm³ were randomized into treatment group and were dosed per individual body weight on Day 0. Mice were monitored for tumor growth around 60 days post dosing and terminated at the end of the study. Mice were euthanized when the tumors reached tumor end point (1500 mm³). The dosing information is summarized in Table 1. Taxotere® was dosed at Q3Dx3 intravenously (iv) through the tail vein. Isotype control antibody Rituxan® and the anti-PSMA 7F12 antibody were dosed intraperitoneally (ip) Q3Dx5, followed with Q7Dx6.

TABLE 1
Dosing Information

| | | Taxotere ® | Antibody | |
|---|---|---|---|---|
| | N per | Dose iv | Dose ip | |
| Treatment | group | (mg/kg) | (mg/kg) | Dosing Schedule |
| PBS | 10 | | | Day 0, 3, 7, 10, 14, 21, 28, 35, 42, 49, 56 |
| Taxotere ® 2 | 10 | 2 | 0 | Day 0, 3, 7 |
| Taxotere ® 4 | 10 | 4 | 0 | Day 0, 3, 7 |
| Isotype Ab Rituxan ® | 10 | 0 | 30 | Day 0, 3, 7, 10, 14, 21, 28, 35, 42, 49, 56 |
| 7F12 Ab | 10 | 0 | 30 | Day 0, 3, 7, 10, 14, 21, 28, 35, 42, 49, 56 |
| Taxotere ® 2 + 7F12 Ab | 10 | 2 | 30 | Day 0, 3, 7 for Taxotere |
| | | | | Day 0, 3, 7, 10, 14, 21, 28, 35, 42, 49, 56 |
| Taxotere 2 + Isotype | 10 | 2 | 30 | Day 0, 3, 7 for Taxotere |
| Rituxan ® | | | | Day 0, 3, 7, 10, 14, 21, 28, 35, 42, 49, 56 |
| Taxotere 4 + 7F12 Ab | 10 | 4 | 30 | Day 0, 3, 7 for Taxotere |
| | | | | Day 0, 3, 7, 10, 14, 21, 28, 35, 42, 49, 56 |
| Taxotere 4 + Isotype | 10 | 4 | 30 | Day 0, 3, 7 for Taxotere |
| Rituxan ® | | | | Day 0, 3, 7, 10, 14, 21, 28, 35, 42, 49, 56 |

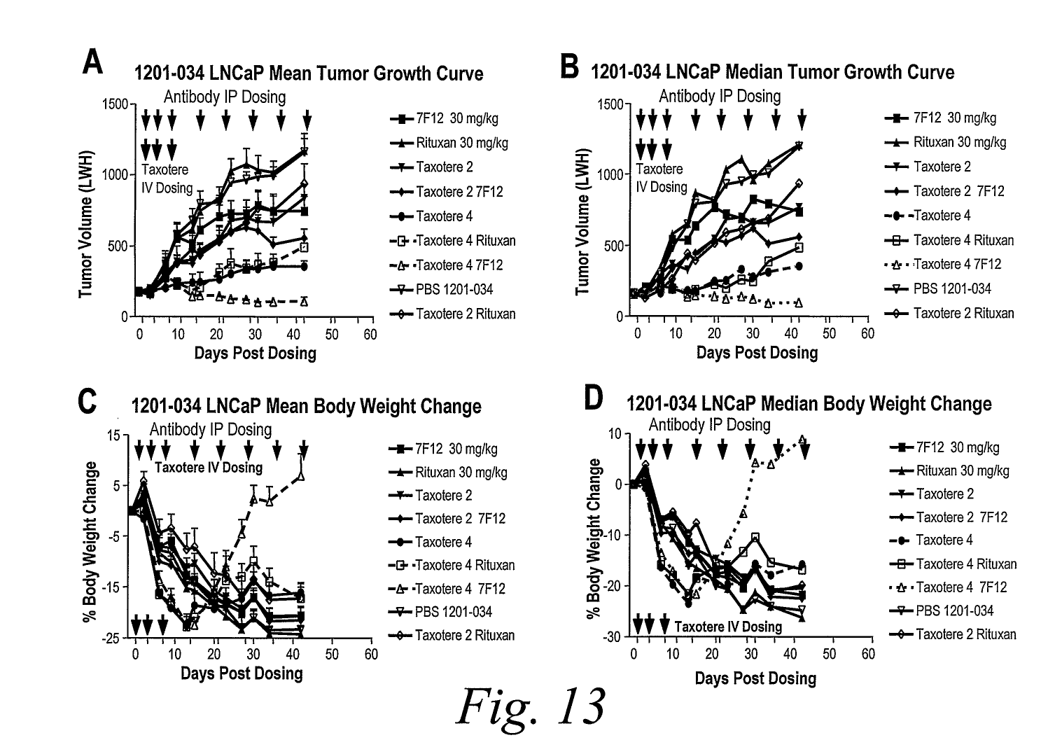

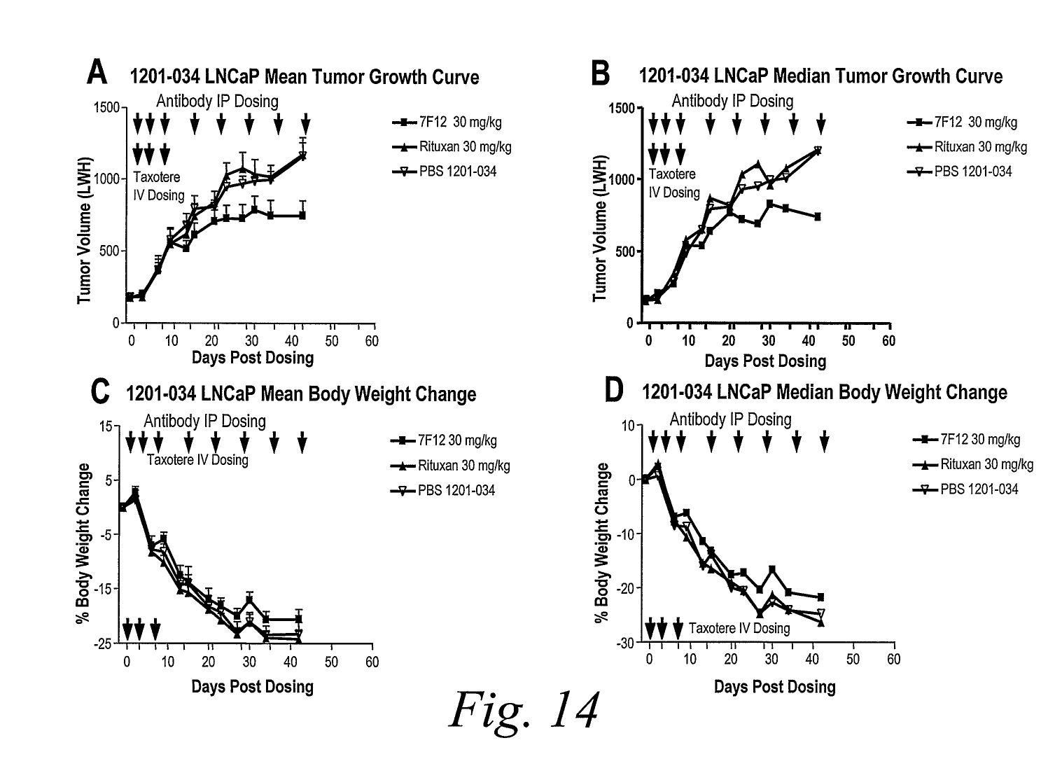

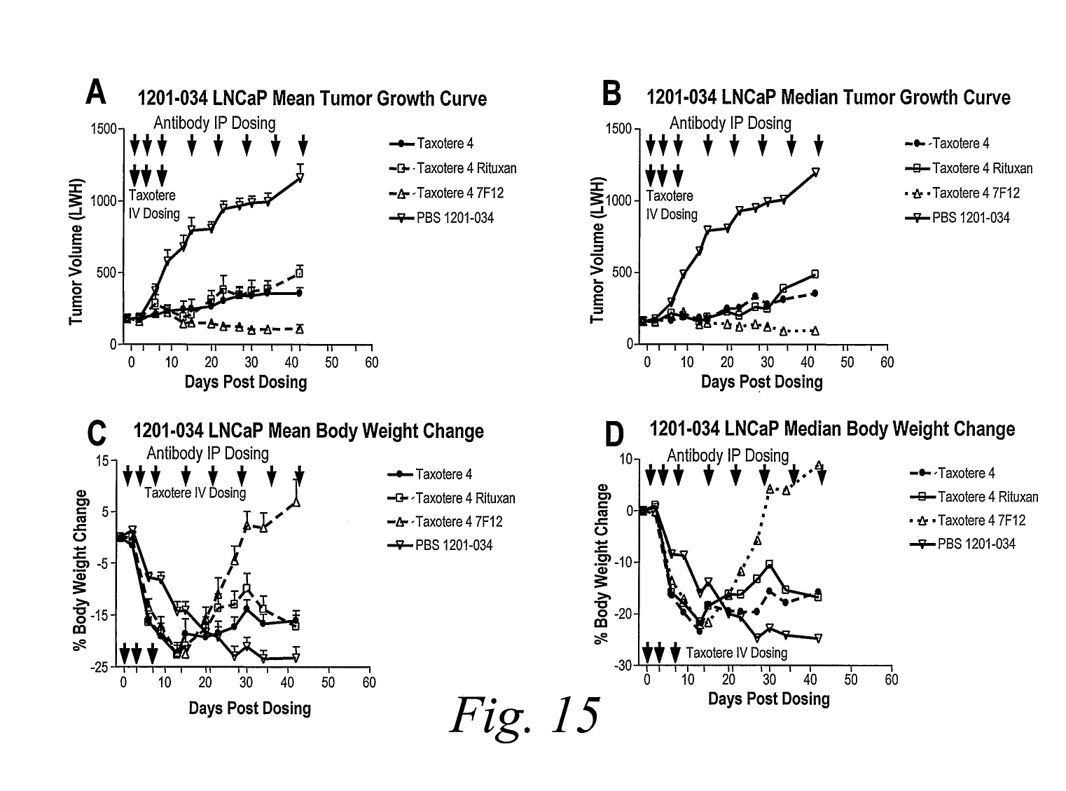

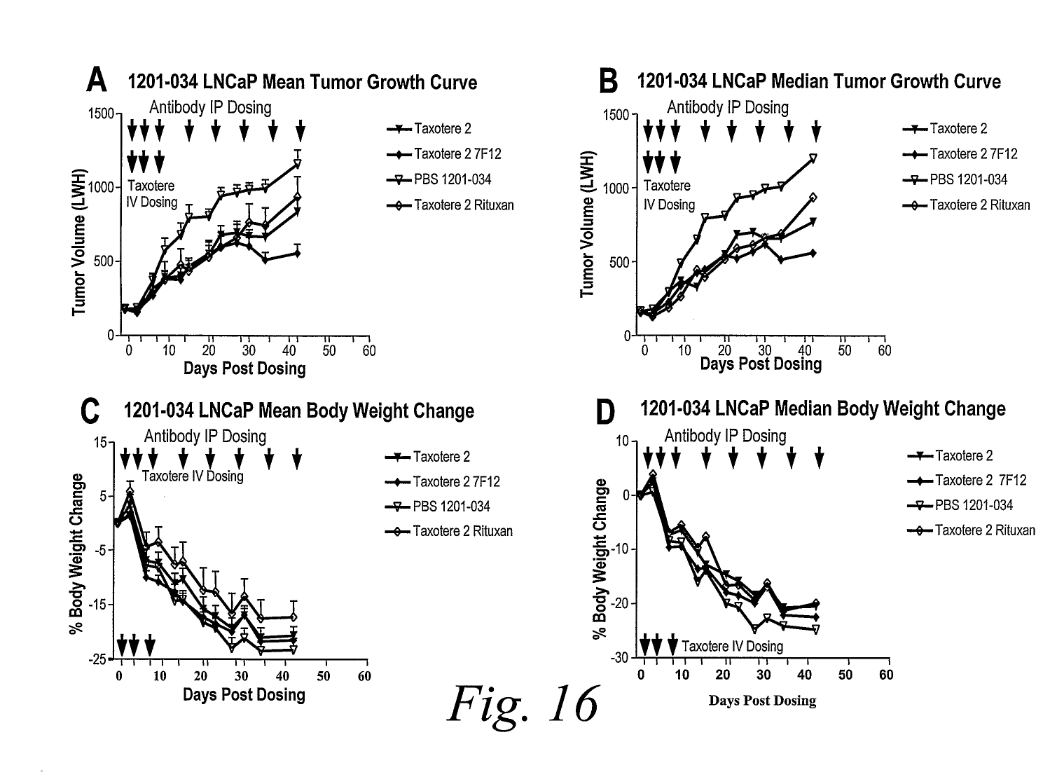

The results of the foregoing experiments are depicted in FIGS. 13-16. As shown in FIGS. 13A-13B and FIGS. 14A-14B, 30 mg/kg of the anti-PSMA 7F12 antibody alone modestly decreased the growth of LNCaP tumors. Taxotere® showed a dose-dependent anti-tumor growth efficacy at the two doses tested (i.e., 2 and 4 mg/kg). Combination therapy of 30 mg/kg of the anti-PSMA 7F12 antibody and 4 mg/kg of Taxotere® showed superior efficacy than each treatment alone and resulted in near complete inhibition of LNCaP tumor growth (see FIGS. 13A-13B and FIGS. 15A-15B). As shown in FIGS. 13C-13D and FIGS. 15C-15D, this combination regimen also cured the mice of LNCaP-tumor-related cachexia. Combination therapy of 30 mg/kg of the anti-PSMA 7F12 antibody and 2 mg/kg of Taxotere® also showed superior efficacy than each treatment alone (see FIGS. 13A-13D and FIGS. 16A-16D). The foregoing data demonstrate an additive and possibly synergistic effect of the anti-PSMA 7F12 antibody in combination with Taxotere® chemotherapy in treating tumors, such as prostate tumors.

### Example 8

### Assessment of Cell Killing of a Toxin-Conjugated Anti-PSMA Antibody on Prostate Cancer Cell Lines

In this example, anti-PSMA monoclonal antibodies conjugated to a toxin were tested for the ability to kill PSMA+prostate cancer cell lines in a cell proliferation assay.

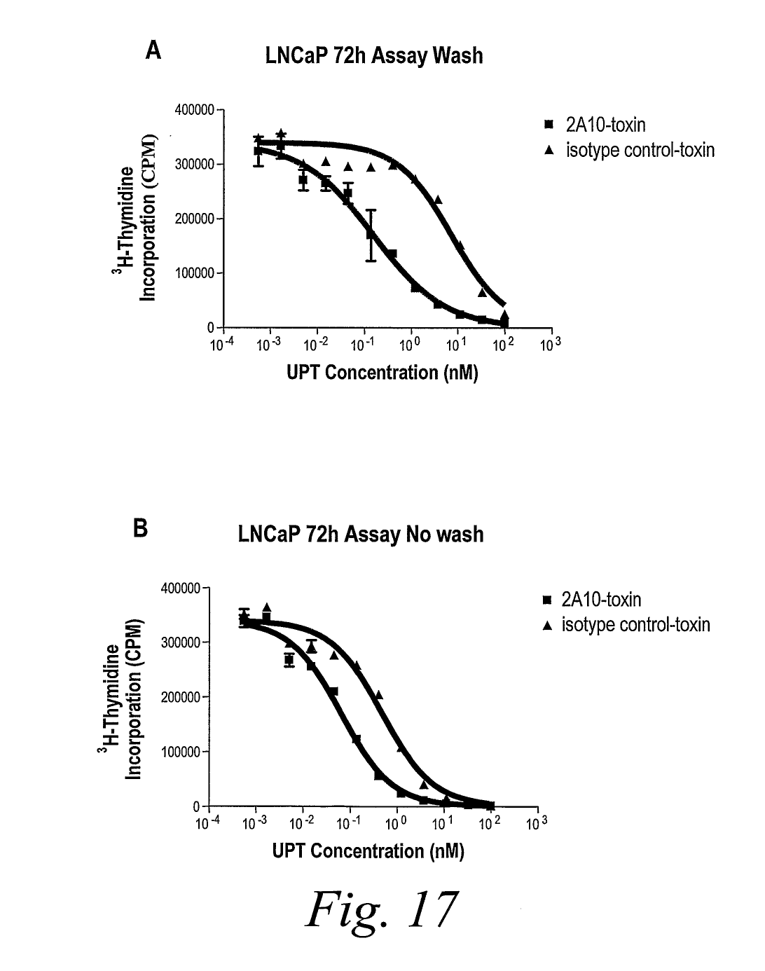

The anti-PSMA HuMAb antibody 2A10 was conjugated to a toxin via a linker, such as a peptidyl, hydrazone or disulfide linker. Examples of toxin compounds that may be conjugated to the antibodies of the current invention are described in the filed application with Attorney Docket No. 04280/100M629US3, filed on Sep. 26, 2005. The PSMA-expressing prostate cancer cell line LNCaP was seeded at 2.5×10⁴ cells/wells in 100 μl wells for 3 hours. An anti-PSMA antibody-toxin conjugate was added to the wells at a starting concentration of 30 nM and titrated down at 1:3 serial dilutions. An isotype control antibody that is non-specific for PSMA was used as a negative control. Plates were allowed to incubate for 72 hours with either a wash at 3 hours or a continuous wash. The plates were then pulsed with 1.0 μCi of ³H-thymidine for 24 hours, harvested and read in a Top Count Scintillation Counter (Packard Instruments, Meriden, Conn.). The results are shown in FIG. 17A (three-hour wash) and 17B (continuous wash). The anti-PSMA antibody 2A10 showed an antibody-toxin concentration dependent decrease in ³H-thymidine incorporation in PSMA-expressing LNCaP prostate cancer cells. The EC₅₀ values for the anti-PSMA antibody 2A10 was 0.157 nM for the wash assay and 0.0643 nM for the continuous wash assay. This data demonstrates that anti-PSMA are cytotoxic to prostate cancer cells when conjugated to a toxin.

### Example 9

### In Vivo Studies

In this example, anti-PSMA monoclonal antibodies conjugated to a toxin were tested for the ability to kill PSMA+ prostate cancer cell lines in vivo.

### A. Treatment of in Vivo Tumor Xenografts

The anti-PSMA HuMAb 2A10 and an isotype control antibody were each buffer exchanged into 0.1M phosphate buffer pH8.0 containing 50 mM NaCl and 2 mM DTPA, and concentrated to 6 mg/ml. Both antibodies were then thiolated by incubation with a 25-fold molar excess of 2-iminothiolane for one hour at room temperature, followed by desalting into 0.1M phosphate buffer pH6.0 containing 50mM NaCl and 2 mM DTPA buffer using a Sephadex G-25 column. Thiolated antibodies were then maintained on ice, whilst the number of thiol groups introduced was determined. This was achieved by reaction of a sample of thiolated antibody with dithiodipyridine (DTDP). The absorbance at 280 nm was measured to determine the concentration of protein in the samples, and then an aliquot of each sample (0.9 ml) was incubated with 0.1 ml DTDP (5mM stock solution in ethanol) for 10 minutes at room temperature. Blank samples of buffer alone plus DTDP were incubated alongside. Absorbance at 324 nm was measured and the number of thiols present per antibody quantitated using an extinction coefficient for thiopyridine of 19800 M⁻¹. In the case of anti-PSMA 5.3 thiols per antibody were introduced, and in the case of the isotype control 6.0.

The thiolated antibodies were then incubated with a 3 fold molar excess of Compound A over the molar concentration of thiol groups.

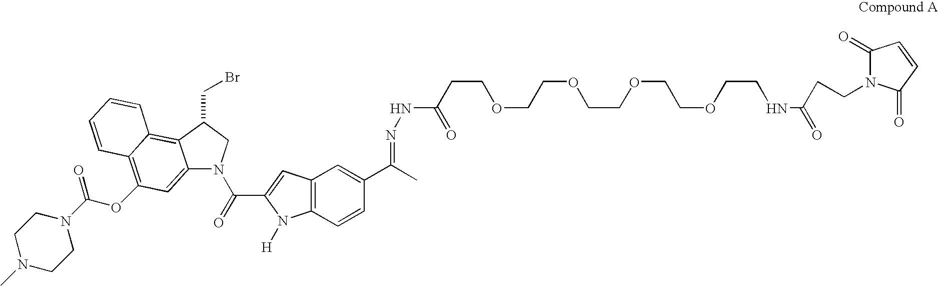

5 mM stock solution in DMSO of Compound A was added to the thiolated antibodies along with sufficient DMSO to bring the final concentration of DMSO to 10% (v/v). After incubation at room temperature for 3 hours the pH of the incubation mixture was raised to 7.0 using triethanolamine. The antibody-Compound A conjugates were then purified by size-exclusion chromatography on a Sephacryl S200 column pre-equilibrated with 0.1M phosphate buffer (pH 7.2) containing 50 mM NaCl and 5% (v/v) DMSO. Fractions containing monomeric conjugate were collected and pooled. The resulting purified conjugates were then concentrated in a stirred cell under nitrogen, using a 10 kDa cut-off membrane. Concentrations and substitution ratios (number of drug molecules attached per antibody molecule) of the conjugates were determined using absorbance at 280 nm and 340 nm, by reference to the extinction coefficients of both antibody and Compound A at each wavelength as previously measured. Examples of other toxin compounds that may be conjugated to the antibodies of the current invention are described in the co-owned U.S. patent application with Attorney Docket No. 04280/100M629US3, filed on Sep. 26, 2005.

Anti-tumor efficacy of anti-PSMA (2A10 clone) conjugated to Compound A was tested on LNCaP, which is human prostate carcinoma xenografts, grown in male CB17.SCID mice (available from Taconic, Germantown, N.Y.). LNCaP prostate cancer cells expressing high levels of PSMA were obtained from ATCC (Cat# CRL-1740) and expanded in vitro following ATCC instruction. 8 week-old male CB17.5CD mice from Taconic were implanted subcutaneously in the right flank with 2.5×10⁶ LNCaP cells in 0.2 ml of PBS/Matrigel (1:1) per mouse. Mice were weighed and measured for tumor three dimensionally using an electronic caliper twice weekly starting three weeks post implantation. Individual tumor volume was calculated as height×width×length. Mice with vascularized tumors (determined by appearance of the tumors) of appropriate sizes were randomized into treatment groups and were dosed per individual body weight on Day 0. Mice were monitored for tumor growth around 60 days post dosing and terminated at the end of the study. Mice were euthanized when the tumors reached tumor end point (1500 mm³). The design of this xenograft study is summarized in table 2.

TABLE 2
LNCaP Xenograft Study Summary

| | | | | Average Tumor |
|---|---|---|---|---|
| | Dose (μmole/kg | N per | Dosing | Volume at |
| Treatment | Cytotoxics) | group | Route | Day −1 (mm³) |
| Vehicle | — | 3 | ip | 100 |
| Isotype Ab- | 0.3 | 3 | ip | 100 |
| Cmpd A | | | | |
| Conjugate | | | | |
| 2A10-Cmpd A | 0.3 | 3 | ip | 100 |
| Conjugate | | | | |

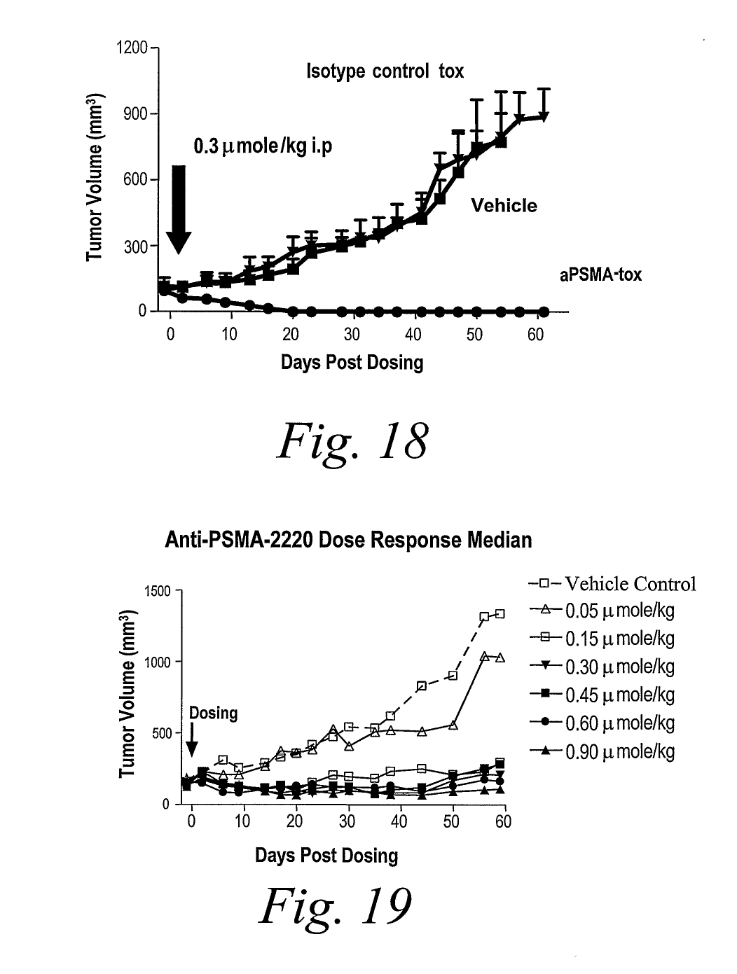

As shown in FIG. 18, 0.3 μmole/kg (referring to the moles of the cytotoxin Compound A) of the 2A10-Compound A conjugate induced complete regression of all three established small LNCaP tumors.

### B. Dose-Response Study

Anti-PSMA (2A10) was buffer exchanged into 0.1M phosphate buffer pH 8.0 containing 50 mM NaCl and 2 mM DTPA, and concentrated to 5.6 mg/nl. Antibody was then thiolated by incubation with a 7.5-fold molar excess of 2-iminothiolane for one hour at room temperature, followed by desalting into 5mM HEPES buffer pH 6.0 containing 5 mM glycine, 2 mM DTPA and 3% (v/v) glycerol using a Sephadex G-25 column. Thiolated antibody was maintained on ice, whilst the number of thiol groups introduced was determined. This was achieved by reaction of a sample of thiolated antibody with dithiodipyridine (DTDP). The absorbance at 280 nm was measured to determine the concentration of protein in the samples, and then an aliquot of each sample (0.9 ml) was incubated with 0.1 ml DTDP (5 mM stock solution in ethanol) for 10 minutes at room temperature. Blank samples of buffer alone plus DTDP were incubated alongside. Absorbance at 324 nm was measured and the number of thiols present per antibody quantitated using an extinction coefficient for thiopyridine of 19800 M⁻¹.

The thiolated antibody was then incubated with a 2-fold molar excess of Compound A over the molar concentration of thiol groups. Compound A, 5 mM stock solution in 10% (v/v) DMSO/90% (v/v) ethylene glycol dimethyl ether, was added to the thiolated antibody along with sufficient ethylene glycol dimethyl ether to bring the final concentration to 5% (v/v). After incubation at room temperature for 2 hours the antibody-Compound A conjugate was purified by ion-exchange chromatography. Reaction mix was applied to an SP-Sepharose column pre-equilibrated in buffer A (50 mM HEPES, 5 mM glycine, 3% (v/v) glycerol, pH 6.0). The column was washed with buffer A, then with 95% buffer A, 5% buffer B (50 mM HEPES, 1M NaCl, 5 mM glycine, 3% (v/v) glycerol, pH 7.2) and then antibody-Compound A conjugate was eluted with 10% buffer B, 90% buffer A. Fractions containing monomeric conjugate were collected and pooled and the pH adjusted to 7.2 by addition of monoethanolamine. The resulting purified conjugate was then dialysed into 50 mM HEPES, 100 mM NaCl, 5 mM glycine, 3% (v/v) glycerol, pH 7.2 and then concentrated in a stirred cell under nitrogen, using a 10 kDa cut-off membrane. Concentrations and substitution ratios (number of drug molecules attached per antibody molecule) of the conjugate was determined using absorbance at 280 nm and 340 nm, by reference to the extinction coefficients of both antibody and Compound A at each wavelength as previously measured. The isotype control (anti-CD70 2H5) conjugate was prepared using the same method except that elution of conjugate from the ion-exchange column was achieved with 15% buffer B, 85% buffer A.

Efficacy and selectivity of the conjugates was determined using LNCaP human prostate carcinoma xenografts grown in male CB17.SCID mice as described above. The design of this xenograft study is summarized in table 3.

TABLE 3
LNCaP Xenograft Study Summary

| | | | | Average Tumor |
|---|---|---|---|---|
| | Dose (μmole/kg | N per | Dosing | Volume at |
| Treatment | Cytotoxin) | group | Route | Day −1 (mm³) |
| Vehicle | — | 9 | ip | 160 |
| Isotype Ab- | 0.05, 0.15, 0.30, | 9 | ip | 160 |
| Cmpd A | 0.45, 0.60, 0.90 | | | |
| 2A10-Cmpd A | 0.05, 0.15, 0.30, | 9 | ip | 160 |
| | 0.45, 0.60, 0.90 | | | |

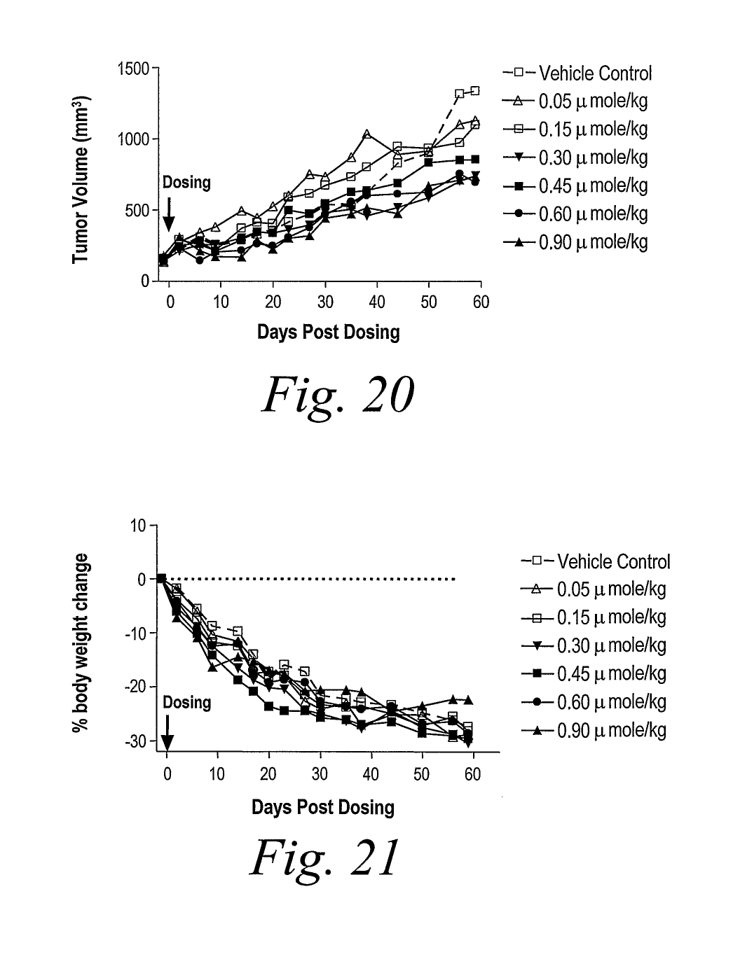

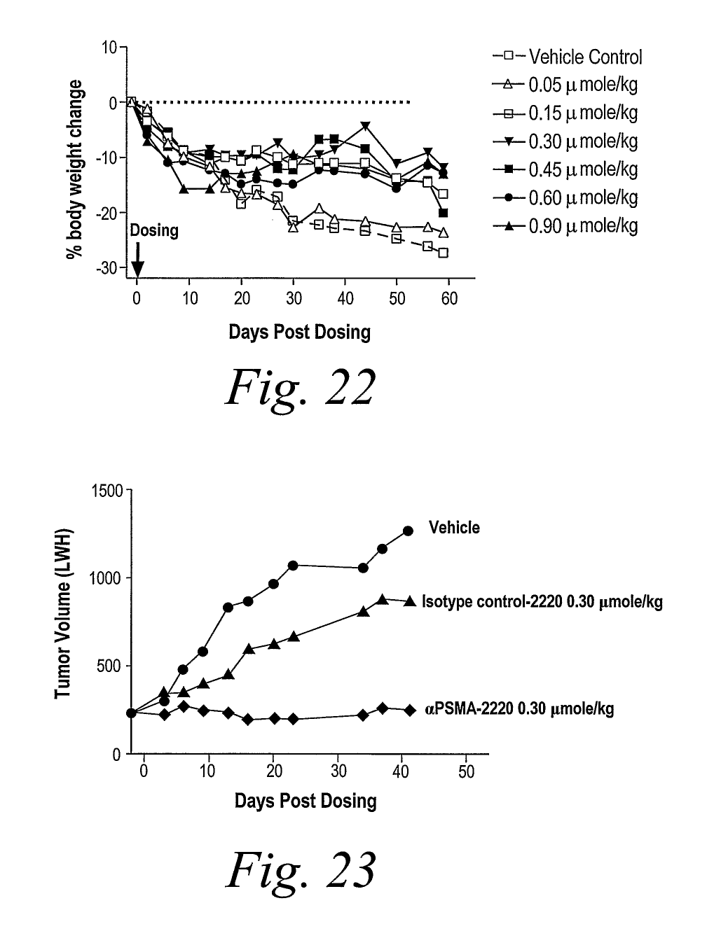

As shown in Table 3 and FIGS. 19-20, 0.15 μmole/kg of anti-PSMA-Compound A (FIG. 19) had better anti-tumor efficacy than 0.90 μmole/kg of isotype control-Compound A, indicating at least >6× selectivity (FIG. 20). 0.90 mmole/kg of anti-PSMA-Compound A only showed transient toxicity (FIGS. 21-22) and was below the maximum tolerated dose. Therefore, an over 6-fold therapeutic index was identified for anti-PSMA-Compound A in LNCaP-tumor-bearing mice.

### C. Efficacy on Large Tumors

Anti-PSMA (2A10) was buffer exchanged into 0.1M phosphate buffer pH8.0 containing 50 mM NaCl and 2 mM DTPA, and concentrated to 5.6 mg/ml. Antibody was then thiolated by incubation with a 9-fold molar excess of 2-iminothiolane for one hour at room temperature, followed by desalting into 50 mM HEPES buffer pH6.0 containing 5 mM glycine, 2 mM DTPA and 3% (v/v) glycerol using a Sephadex G-25 column. Thiolated antibody was maintained on ice, whilst the number of thiol groups introduced was determined. This was achieved by reaction of a sample of thiolated antibody with dithiodipyridine (DTDP). The absorbance at 280 nm was measured to determine the concentration of protein in the samples, and then an aliquot of each sample (0.9 ml) was incubated with 0.1 ml DTDP (5 mM stock solution in ethanol) for 10 minutes at room temperature. Blank samples of buffer alone plus DTDP were incubated alongside. Absorbance at 324 nm was measured and the number of thiols present per antibody quantitated using an extinction coefficient for thiopyridine of 19800 M⁻¹.

The thiolated antibody was then incubated with a 2-fold molar excess of Compound A over the molar concentration of thiol groups. Compound A, 5 mM stock solution in 10% (v/v) DMSO 90% (v/v) ethylene glycol dimethyl ether, was added to the thiolated antibody along with sufficient ethylene glycol dimethyl ether to bring the final concentration to 5% (v/v). After incubation at room temperature for 2 hours the antibody-Compound A conjugate was purified by ion-exchange chromatography. Reaction mix was applied to an SP-Sepharose column pre-equilibrated in 50 mM HEPES, 5 mM glycine, 3% (v/v) glycerol, pH 6.0 (buffer A). The column was washed with buffer A, then with 95% buffer A, 5% buffer B (50 mM HEPES, 1M NaCl, 5 mM glycine, 3% (v/v) glycerol, pH 7.2) and then antibody-Compound A conjugate was eluted with 10% buffer B, 90% buffer A. Fractions containing monomeric conjugate were collected and pooled and the pH adjusted to 7.2 by addition of monoethanolamine. The resulting purified conjugate was then dialysed into 50 mM HEPES, 100 mM NaCl, 5 mM glycine, 3% (v/v) glycerol, pH 7.2 and then concentrated in a stirred cell under nitrogen, using a 10 kDa cut-off membrane. Concentrations and substitution ratios (number of drug molecules attached per antibody molecule) of the conjugate was determined using absorbance at 280 nm and 340 nm, by reference to the extinction coefficients of both antibody and Compound A at each wavelength as previously measured. The isotype control (anti-CD70 2H5) conjugate was prepared using the same method except that elution of conjugate from the ion-exchange column was achieved with 15% buffer B, 85% buffer A.

Efficacy and selectivity of the conjugates was determined using LNCaP human prostate carcinoma xenografts grown in male CB17.SCID mice as described above. The design of these xenograft studies is summarized in tables 4 & 5.

TABLE 4
LNCaP Xenograft Study Summary

| | | | | Average Tumor |
|---|---|---|---|---|
| | Dose (μmole/kg | N per | Dosing | Volume at |
| Treatment | Cytotoxin) | group | Route | Day −1 (mm³) |
| Vehicle | — | 8 | iv | 240 |
| Isotype Ab- | 0.15 | 8 | iv | 240 |
| Cmpd A | | | | |
| 2A10-Cmpd A | 0.15 | 8 | iv | 240 |

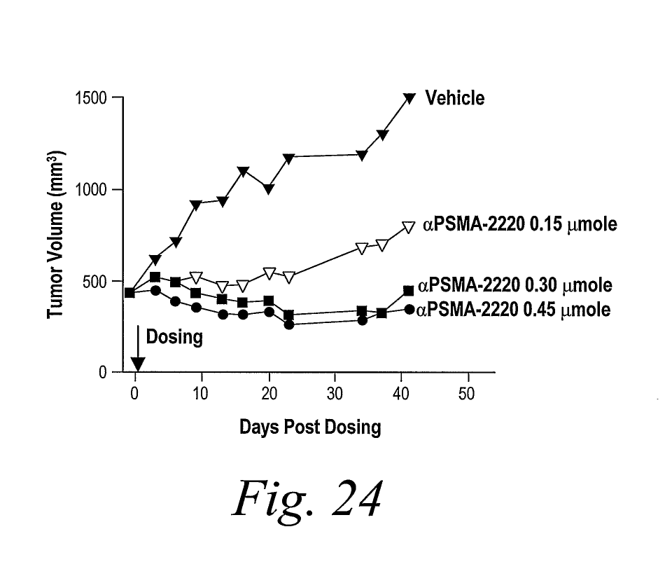

As shown in Table 4 and FIG. 23, a single low dose of 0.15 mmole/kg of anti-PSMA-Compound A greatly inhibited growth of established large LNCaP tumors of average sizes of 240 mm³. In contrast, 0.15 μmole/kg of isotype control-Compound A had minimal anti-tumor efficacy. As shown in Table 5 and FIG. 24, a single dose of 0.30 μmole/kg of anti-PSMA-Compound A induced regression and inhibited growth of very large LNCaP tumors of average sizes of 430 mm³.

TABLE 5
LNCaP Xenograft Study Summary

| | | | | Average Tumor |
|---|---|---|---|---|
| | Dose (μmole/kg | N per | Dosing | Volume at |
| Treatment | Cytotoxin) | group | Route | Day −1 (mm³) |
| Vehicle | — | 6 | ip | 430 |
| 2A10-Cmpd A | 0.15, 0.30, 0.45 | 6 | ip | 430 |

Each of the patent applications, patents, publications, and other published documents mentioned or referred to in this specification is herein incorporated by reference in its entirety, to the same extent as if each individual patent application, patent, publication, and other published document was specifically and individually indicated to be incorporated by reference.

While the present invention has been described with reference to the specific embodiments thereof, it should be understood by those skilled in the art that various changes may be made and equivalents may be substituted without departing from the true spirit and scope of the invention and the appended claims. In addition, many modifications may be made to adapt a particular situation, material, composition of matter, process, process step or steps, to the objective, spirit and scope of the present invention. All such modifications are intended to be within the scope of the claims appended hereto.

SUMMARY OF SEQUENCE LISTING

| SEQ ID | | SEQ ID | |
|---|---|---|---|
| NO: | SEQUENCE | NO: | SEQUENCE |
| 1 | VH a.a. 1C3 | 21 | VK CDR1 a.a. 1C3 |
| 2 | VH a.a. 2A10 | 22 | VK CDR1 a.a. 2A10 |
| 3 | VH a.a. 2F5 | 23 | VK CDR1 a.a. 2F5 |
| 4 | VH a.a. 2C6 | 24 | VK CDR1 a.a. 2C6 |
| 5 | VK a.a. 1C3 | 25 | VK CDR2 a.a. 1C3 |
| 6 | VK a.a. 2A10 | 26 | VK CDR2 a.a. 2A10 |
| 7 | VK a.a. 2F5 | 27 | VK CDR2 a.a. 2F5 |
| 8 | VK a.a. 2C6 | 28 | VK CDR2 a.a. 2C6 |
| 9 | VH CDR1 a.a. 1C3 | 29 | VK CDR3 a.a. 1C3 |
| 10 | VH CDR1 a.a. 2A10 | 30 | VK CDR3 a.a. 2A10 |
| 11 | VH CDR1 a.a. 2F5 | 31 | VK CDR3 a.a. 2F5 |
| 12 | VH CDR1 a.a. 2C6 | 32 | VK CDR3 a.a. 2C6 |
| 13 | VH CDR2 a.a. 1C3 | 33 | VH n.t. 1C3 |
| 14 | VH CDR2 a.a. 2A10 | 34 | VH n.t. 2A10 |
| 15 | VH CDR2 a.a. 2F5 | 35 | VH n.t. 2F5 |
| 16 | VH CDR2 a.a. 2C6 | 36 | VH n.t. 2C6 |
| 17 | VH CDR3 a.a. 1C3 | 37 | VK n.t. 1C3 |
| 18 | VH CDR3 a.a. 2A10 | 38 | VK n.t. 2A10 |
| 19 | VH CDR3 a.a. 2F5 | 39 | VK n.t. 2F5 |
| 20 | VH CDR3 a.a. 2C6 | 40 | VK n.t. 2C6 |
| 41 | VH 3-30.3 germline a.a. | 43 | VK L18 germline a.a. |
| 42 | VH 5-51 germline a.a. | 44 | VK L6 |
| 45 | JH6b germline a.a. | 47 | JK3 germline a. a. |
| 46 | JK4 germline a.a. | 48 | (Gly₄-Ser)₃ a.a. |

We claim:

1. An isolated human monoclonal antibody that specifically binds prostate specific membrane antigen (PSMA), wherein the antibody has a melting temperature of at least 67° C.

2. The antibody of claim 1, which has a melting temperature of at least 69° C.

3. The antibody of claim 1, which has a melting temperature of at least 71° C.

4. An isolated monoclonal antibody, or an antigen-binding portion thereof, comprising a heavy chain variable region that is the product of or derived from a human V_{H} 3-30.3 gene, wherein the antibody specifically binds PSMA.

5. An isolated monoclonal antibody, or an antigen-binding portion thereof, comprising:
a) a heavy chain variable region of a human V_{H} 3-30.3 gene; and
b) a light chain variable region of a human Vk L18 gene;
wherein the antibody specifically binds PSMA.

6. An isolated monoclonal antibody, or an antigen-binding portion thereof, comprising a light chain variable region that is the product of or derived from a human V_{K} L18 gene, wherein the antibody specifically binds PSMA.

7. An isolated monoclonal antibody, or an antigen-binding portion thereof, comprising:
a) a heavy chain variable region of a human V_{H} 5-51 gene; and
b) a light chain variable region of a human Vk L18 gene;
wherein the antibody specifically binds PSMA.

8. An isolated monoclonal antibody, or antigen binding portion thereof, comprising:
a) a heavy chain variable region CDR1 comprising an amino acid sequence selected from the group consisting of SEQ ID NOs: 9, 10, 11, and 12;
b) a heavy chain variable region CDR2 comprising an amino acid sequence selected from the group consisting of SEQ ID NOs: 13, 14, 15, and 16;
c) a heavy chain variable region CDR3 comprising an amino acid sequence selected from the group consisting of SEQ ID NOs: 17, 18, 19, and 20;
d) a light chain variable region CDR1 comprising an amino acid sequence selected from the group consisting of SEQ ID NOs: 21, 22, 23, and 24;
e) a light chain variable region CDR2 comprising an amino acid sequence selected from the group consisting of SEQ ID NOs: 25, 26, 27, and 28; and
f) a light chain variable region CDR3 comprising an amino acid sequence selected from the group consisting of SEQ ID NOs: 29, 30, 31, and 32;
wherein the antibody specifically binds PSMA.

9. The antibody of claim 8, which comprises:
a) a heavy chain variable region CDR1 comprising SEQ ID NO: 9;
b) a heavy chain variable region CDR2 comprising SEQ ID NO: 13;
c) a heavy chain variable region CDR3 comprising SEQ ID NO: 17;
d) a light chain variable region CDR1 comprising SEQ ID NO: 21;
e) a light chain variable region CDR2 comprising SEQ ID NO: 25; and
f) a light chain variable region CDR3 comprising SEQ ID NO: 29.

10. The antibody of claim 8, which comprises:
a) a heavy chain variable region CDR1 comprising SEQ ID NO: 10;
b) a heavy chain variable region CDR2 comprising SEQ ID NO: 14;
c) a heavy chain variable region CDR3 comprising SEQ ID NO: 18;
d) a light chain variable region CDR1 comprising SEQ ID NO: 22;
e) a light chain variable region CDR2 comprising SEQ ID NO: 26; and
f) a light chain variable region CDR3 comprising SEQ ID NO: 30.

11. The antibody of claim 8, which comprises:
a) a heavy chain variable region CDR1 comprising SEQ ID NO: 11;
b) a heavy chain variable region CDR2 comprising SEQ ID NO: 15;
c) a heavy chain variable region CDR3 comprising SEQ ID NO: 19;
d) a light chain variable region CDR1 comprising SEQ ID NO: 23;
e) a light chain variable region CDR2 comprising SEQ ID NO: 27; and
f) a light chain variable region CDR3 comprising SEQ ID NO: 31.

12. The antibody of claim 8, which comprises:
a) a heavy chain variable region CDR1 comprising SEQ ID NO: 12;
b) a heavy chain variable region CDR2 comprising SEQ ID NO: 16;
c) a heavy chain variable region CDR3 comprising SEQ ID NO: 20;
d) a light chain variable region CDR1 comprising SEQ ID NO: 24;
e) a light chain variable region CDR2 comprising SEQ ID NO: 28; and
f) a light chain variable region CDR3 comprising SEQ ID NO: 32.

13. An isolated monoclonal antibody, or antigen binding portion thereof comprising:
a) a heavy chain variable region comprising an amino acid sequence selected from the group consisting of SEQ ID NOs: 1, 2, 3, and 4; and
b) a light chain variable region comprising an amino acid sequence selected from the group consisting of SEQ ID NOs: 5, 6, 7, and 8;
wherein the antibody specifically binds PSMA.

14. The antibody of claim 13, which comprises:
a) a heavy chain variable region comprising the amino acid sequence of SEQ ID NO: 1; and
b) a light chain variable region comprising the amino acid sequence of SEQ ID NO: 5.

15. The antibody of claim 13, which comprises:
a) a heavy chain variable region comprising the amino acid sequence of SEQ ID NO: 2; and
b) a light chain variable region comprising the amino acid sequence of SEQ ID NO: 6.

16. The antibody of claim 13, which comprises:
a) a heavy chain variable region comprising the amino acid sequence of SEQ ID NO: 3; and
b) a light chain variable region comprising the amino acid sequence of SEQ ID NO: 7.

17. The antibody of claim 13, which comprises:
a) a heavy chain variable region comprising the amino acid sequence of SEQ ID NO: 4; and
b) a light chain variable region comprising the amino acid sequence of SEQ ID NO: 8.

18. A composition comprising the antibody, or antigen-binding portion thereof, of any one of claims 1-17, and a pharmaceutically acceptable carrier.

19. An immunoconjugate comprising the antibody, or antigen-binding portion thereof, of any one of claims 1-17, linked to a therapeutic agent.

20. A composition comprising the immunoconjugate of claim 19 and a pharmaceutically acceptable carrier.

21. The immunoconjugate of claim 19, wherein the therapeutic agent is a cytotoxin.

22. A composition comprising the immunoconjugate of claim 21 and a pharmaceutically acceptable carrier.

23. The immunoconjugate of claim 19, wherein the therapeutic agent is a radioactive isotope.

24. A composition comprising the immunoconjugate of claim 23 and a pharmaceutically acceptable carrier.

25. A bispecific molecule comprising the antibody, or antigen-binding portion thereof, of any one of claims 1-17, linked to a second functional moiety having a different binding specificity than said antibody, or antigen binding portion thereof.

26. A composition comprising the bispecific molecule of claim 25, and a pharmaceutically acceptable carrier.

27. An isolated nucleic acid molecule encoding the antibody, or antigen-binding portion thereof, of any one of claims 1-17.

28. An expression vector comprising the nucleic acid molecule of claim 27.

29. A host cell comprising the expression vector of claim 28.

30. A transgenic mouse comprising human immunoglobulin heavy and light chain transgenes, wherein the mouse expresses the antibody of any one of claims 1-17.

31. A hybridoma prepared from the mouse of claim 30, wherein the hybridoma produces said antibody.

32. A method of inhibiting growth of a tumor in a subject, wherein cells of the tumor or vascular endothelial cells proximate to the tumor express PSMA, comprising administering to a subject the antibody, or antigen-binding portion thereof, of any one of claims 1-17 in an amount effective to inhibit growth of the tumor.

33. The method of claim 32, wherein the tumor is a prostate cancer tumor.

34. The method of claim 32, wherein the tumor is of a cancer selected from the group consisting of colon, renal, rectal, urothelial, breast, bladder, liver, pancreas or melanoma.

35. A method for preparing an anti-PSMA antibody comprising:
(a) providing: (i) a heavy chain variable region antibody sequence comprising a CDR1 sequence that is selected from the group consisting of SEQ ID NOs:
9, 10, 11, and 12, a CDR2 sequence that is selected front the group consisting of SEQ ID NOs: 13, 14, 15, and 16; and a CDR3 sequence that is selected from the group consisting of SEQ ID NOs: 17, 18, 19, and 20; or (ii) a light chain variable region antibody sequence comprising a CDR1 sequence that is selected from the group consisting of SEQ ID NOs: 21, 22, 23, and 24, a CDR2 sequence that is selected from the group consisting of SEQ ID NOs: 25, 26, 27, and 28, and a CDR3 sequence that is selected from the group consisting of SEQ ID NOs: 29, 30, 31, and 32;
(b) altering at least one amino acid residue within at least one variable region antibody sequence, said sequence being selected from the heavy chain variable region antibody sequence and the light chain variable region antibody sequence, to create at least one altered antibody sequence; and
(c) expressing the altered antibody sequence as a protein.

36. A method of inhibiting or preventing growth of a tumor in a subject, wherein cells of the tumor or vascular endothelial cells proximate to the tumor express PSMA, comprising administering to a subject an anti-PSMA antibody, or antigen-binding portion thereof, in combination with an anti-tumor agent both in an amount effective to inhibit or prevent growth of the tumor.

37. The method of claim 36, wherein the administration to said subject of said anti-PSMA antibody, or antigen-binding portion thereof, in combination with said anti-tumor agent, leads to a synergistic effect on the inhibition of the growth of said tumor.

38. The method of claim 36, wherein said anti-tumor agent causes damage in the tumor mass, thereby leading to a more effective antibody dependent cell-mediated cytotoxicity (ADCC) of the tumor.

39. The method of claim 36, wherein said anti-PSMA antibody is the antibody of any one of claims 1-17.

40. The method of claim 36, wherein said anti-PSMA antibody is the 7F12 antibody.

41. The method of claim 36, wherein said anti-PSMA antibody is the 2A10 antibody.

42. The method of claim 36, wherein the tumor is a prostate cancer tumor.

43. The method of claim 36, wherein the tumor is of a cancer selected from the group consisting of colon, renal, rectal, urothelial, breast, bladder, liver, pancreas and melanoma.

44. The method of claim 36, wherein said anti-tumor agent is a chemotherapeutic agent.

45. The method of claim 44, wherein, said chemotherapeutic agent is Taxotere® (docetaxel).

46. The method of claim 36, wherein said anti-tumor agent is an anti-angiogenic agent.

47. The method of claim 46, wherein said anti-angiogenic agent is selected from the group consisting of angiostatin K1-3, Arresten, aaAT, Canstatin, DL-α-Difluoromethyl-ornithine, Endostatin, Fumagillin, Genistein, Minocycline, Staurosporine, Thalidomide, and Tumstatin.

48. The method of claim 36, wherein said anti-tumor agent is an immunomodulatory agent.

49. The method of claim 48, wherein said immunomodulatory agent is selected from the group consisting of anti-PD1 antibodies, anti-CTLA-4 antibodies, phosphorothiolate oligodeoxyribonucleotide (1018 ISS), GM-CSF gene vaccines, interleukin-2, interleukin-7 (CYT 99 07), interleukin-12 and interleukin-21.

50. A method of stimulating antibody dependent cell-mediated cytotoxicity (ADCC) of a tumor in a subject, wherein cells of the tumor or vascular endothelial cells proximate to the tumor express PSMA, comprising administering to a subject an anti-PSMA antibody, or antigen-binding portion thereof, in combination with an anti-tumor agent both in an amount effective to stimulate antibody dependent cell-mediated cytotoxicity (ADCC) of the tumor.

51. A method of inhibiting tumor-related cachexia in a subject, wherein cells of the tumor or vascular endothelial cells proximate to the tumor express PSMA, comprising administering to a subject an anti-PSMA antibody, or antigen-binding portion thereof, in combination with an anti-tumor agent both in an amount effective to inhibit tumor-related cachexia in the subject.

52. A composition comprising an anti-PSMA antibody and an anti-tumor agent in an amount effective to inhibit or prevent growth of a tumor and a pharmaceutically acceptable carrier.

53. A composition comprising an anti-PSMA antibody and an anti-tumor agent in an amount effective to stimulate antibody dependent cell-mediated cytotoxicity (ADCC) of a tumor and a pharmaceutically acceptable carrier.

54. A method of identifying an anti-tumor agent capable of acting synergistically with an anti-PSMA antibody in inhibiting or preventing growth of a tumor, wherein cells of the tumor or vascular endothelial cells proximate to the tumor express PSMA, comprising:
contacting an indicator composition with:
(a) a test anti-tumor agent alone,
(b) an anti-PSMA antibody alone, and
(c) both a test anti-tumor agent and an anti-PSMA antibody; and
comparing the ability of (a) the test anti-tumor agent alone and (b) the anti-PSMA antibody alone to inhibit or prevent growth of a tumor to the ability of (c) both the test anti-tumor agent and the anti-PSMA antibody to inhibit or prevent growth of a tumor, wherein inhibition or prevention of tumor growth by (c) in an amount that is greater than the additive effect of (a) and (b) would lead to the identification of an anti-tumor agent capable of acting synergistically with an anti-PSMA antibody in inhibiting or preventing growth of a tumor.

55. The method of claim 54, wherein the indicator composition is an animal model for a tumor.
